(19) United States
(12) Patent Application Publication
(10) Pub. No.: US 2023/0012607 A1
(43) Pub. Date: Jan. 19, 2023

(54) METHODS FOR DETECTING ANALYTES USING SPARSE LABELLING

(71) Applicant: 10x Genomics, Inc., Pleasanton, CA (US)

(72) Inventors: Malte KÜHNEMUND, Stockholm (SE); Xiaoyan QIAN, Stockholm (SE); Toon VERHEYEN, Solna (SE)

(21) Appl. No.: 17/860,960

(22) Filed: Jul. 8, 2022

Related U.S. Application Data
(60) Provisional application No. 63/220,412, filed on Jul. 9, 2021.
(60) Provisional application No. 63/306,855, filed on Feb. 4, 2022.

(51) Int. Cl. C12Q 1/6869; C12Q 1/6841; C12Q 1/6832; G01N 21/64

(52) U.S. Cl. CPC C12Q 1/6869; C12Q 1/6841; C12Q 1/6832; G01N 21/6458; G01N 21/6428; G01N 2021/6439

(57) ABSTRACT

In some aspects, the present disclosure relates to methods for reducing the crowding of signals, for example optical crowding, that can occur when nucleic acids are detected in a sample in multiplex, which can make it difficult to resolve individual signals and can lead to a reduced dynamic range. In some aspects, the present disclosure relates to methods for reducing signal crowding in the detection of multiple target nucleic acid sequences in a sample, e.g., using hybridization probes, wherein signal crowding from said hybridization probes is reduced. The methods herein have particular applicability in the detection of barcode sequences by sequencing-by-hybridization (SBH) methods, including those relying on combinatorial labelling schemes and decoding of the barcodes by sequential cycles of decoding using hybridization probes. Also provided are kits comprising probes for use in such methods.

### CROSS-REFERENCE TO RELATED APPLICATIONS

This application claims priority to U.S. Provisional Patent Application No. 63/220,412, filed Jul. 9, 2021, entitled “METHODS FOR DETECTING ANALYTES USING SPARSE LABELLING,” and to U.S. Provisional Patent Application No. 63/306,855, filed Feb. 4, 2022, entitled “METHODS FOR DETECTING ANALYTES USING SPARSE LABELLING,” each of which is herein incorporated by reference in its entirety for all purposes.

### FIELD

The present disclosure generally relates to methods and compositions for detecting a plurality of molecules of one or more analytes in a sample.

### BACKGROUND

In multiplex assays where multiple signals are detected simultaneously, it is important that each individual signal can be distinguished from one another, so that as much information as possible can be collected from the assays. For example, in microscopy-based optical assays, individual “spots” emitting optical signals often need to be resolved from adjacent spots in a sample. However, resolving a large number of signals of varying strengths remains challenging, and improved methods are needed. The present disclosure addresses these and other needs.

### SUMMARY

During in situ analysis such as those involving sequencing-by-hybridization (SBH), highly expressed analytes can produce many locally amplified probes in close proximity, causing optical crowding and limiting the dynamic range for quantification. Signal spots (e.g., due to high analyte abundance) may overlap with one another and/or mask adjacent signal spots, rendering the spots unresolvable. In some cases, large spots and relatively smaller spots may overlap and the smaller spots may be masked by the larger spots. In addition, when bright spots and relatively dim spots are in the same microscope field of view, the dim spots may not pass the threshold of spot detection for subsequent image analysis. Thus, highly abundant analytes may not only render detection of the analytes themselves challenging, but also lead to inability to detect nearby signal spots and/or weaker signal spots in the same field of view. As such, improved methods for precise detection and accurate quantification of the expression levels of highly expressed genes in a biological sample are needed. In some aspects, the present disclosure relates to methods and compositions for more accurately detecting and quantifying analytes that may produce overlapping signals, including highly expressed or abundant targets (e.g., genes) in a sample.

In some embodiments, provided herein is a method for analyzing a biological sample comprising a plurality of analytes, comprising: (a) detecting a plurality of optical signals in sequential cycles, wherein at least a subset of the plurality of optical signals detected at a location in the biological sample form an optical signature corresponding to an analyte of the plurality of analytes, the detecting comprising: i) in a first cycle, detecting a plurality of overlapping optical signals at a location; ii) in a second cycle, detecting a first non-overlapping optical signal at the location, wherein the first non-overlapping optical signal is associated with a first analyte; and iii) in a third cycle, detecting a second non-overlapping optical signal at the location, wherein the second non-overlapping optical signal is associated with a second analyte; and (b) using an identifier for the first analyte and an identifier for the second analyte to associate a first overlapping optical signal of the plurality overlapping optical signals to the first analyte, and associate a second overlapping optical signal of the plurality overlapping optical signals to the second analyte, wherein the identifier for the first analyte is a first order of signal codes that identifies the first analyte, and the identifier for the second analyte is a second order of signal codes that identifies the second analyte, and wherein the first order comprises signal codes that match an optical signature comprising the first overlapping optical signal and the first non-overlapping optical signal, and the second order comprises signal codes that match an optical signature comprising the second overlapping optical signal and the second non-overlapping optical signal, thereby identifying the first and second analytes at the location.

In some embodiments, disclosed herein is a method for analyzing a biological sample comprising a plurality of analytes, comprising: (a) detecting a plurality of optical signals in sequential cycles, wherein at least a subset of the plurality of optical signals detected at a location in the biological sample form an optical signature corresponding to an analyte of the plurality of analytes, the detecting comprising: i) in a first cycle, detecting a plurality of overlapping optical signals at a location; ii) in a second cycle, detecting a first non-overlapping optical signal at the location, wherein the first non-overlapping optical signal is associated with a first analyte; and iii) in a third cycle, detecting a second non-overlapping optical signal at the location, wherein the second non-overlapping optical signal is associated with a second analyte; and (b) generating a plurality of potential signal sequence chains comprising optical signals from each of the cycles from (a); and (c) using an identifier for the first analyte and an identifier for the second analyte to associate a first potential signal sequence chain of the plurality of potential signal sequence chains to the first analyte, and associate a second potential signal sequence chain of the plurality of potential signal sequence chains to the second analyte, wherein the identifier for the first analyte is a first order of signal codes that identifies the first analyte, and the identifier for the second analyte is a second order of signal codes that identifies the second analyte, and wherein the first order comprises signal codes that match an optical signature comprising a first overlapping optical signal of the plurality of overlapping optical signals and the first non-overlapping optical signal, and the second order comprises signal codes that match an optical signature comprising a second overlapping optical signal of the plurality of overlapping optical signals and the second non-overlapping optical signal, thereby identifying the first and second analytes at the location.

In some embodiments, a probability of matching the identifier for the first or second analyte is assigned to each potential signal sequence chain of the plurality of potential signal sequence chains. In some embodiments, the method can comprise comparing the plurality of potential signal sequence chains to the identifier for the first analyte and the identifier for the second analyte to assign the probability. In any of the preceding embodiments, the plurality of potential signal sequence chains generated in step (b) can comprise the presence and absence of signals detected from step (a). In any of the preceding embodiments, the method can comprise associating the first non-overlapping optical signal at the location with the first overlapping optical signal of the plurality of overlapping optical signals at the location to generate the first potential signal sequence chain. In any of the preceding embodiments, the method can comprise associating the second non-overlapping optical signal at the location with the second overlapping optical signal of the plurality of overlapping optical signals at the location to generate the second potential signal sequence chain.

In any of the preceding embodiments, the optical signals can be detected by detecting detectable probes targeting the plurality of analytes. In any of the preceding embodiments, the optical signals can be detected by imaging the biological sample using fluorescent microscopy. In any of the preceding embodiments, the optical signals can be detected by: contacting the biological sample with one or more detectably-labeled probes that directly or indirectly bind to nucleic acid sequences in or associated with the plurality of analytes, and removing the one or more detectably-labeled probes from the nucleic acid sequences. In some instances, the binding and removing steps are repeated with the one or more detectably-labeled probes and/or one or more other detectably-labeled probes that directly or indirectly bind to nucleic acid sequences in or associated with the plurality of analytes. In any of the preceding embodiments, the optical signals can be detected in situ in the biological sample. In any of the preceding embodiments, the optical signals can be detected by: contacting the biological sample with one or more detectably-labeled probes that directly or indirectly hybridize to nucleic acid sequences in or associated with the plurality of analytes, and dehybridizing the one or more detectably-labeled probes from the nucleic acid sequences. In some instances, the contacting and dehybridizing steps are repeated with the one or more detectably-labeled probes and/or one or more other detectably-labeled probes that directly or indirectly hybridize to nucleic acid sequences in or associated with the plurality of analytes.

In any of the preceding embodiments, the optical signals can be detected by contacting the biological sample with one or more intermediate probes that directly or indirectly bind to nucleic acid sequences in or associated with the plurality of analytes, wherein the one or more intermediate probes are detectable using one or more detectably-labeled probes, and removing the one or more intermediate probes and/or the one or more detectably-labeled probes from the nucleic acid sequences. In some instances, the binding and removing steps are repeated with the one or more intermediate probes, the one or more detectably-labeled probes, one or more other intermediate probes, and/or one or more other detectably-labeled probes. In any of the preceding embodiments, the optical signals can be detected by contacting the biological sample with one or more intermediate probes that directly or indirectly hybridize to nucleic acid sequences in or associated with the plurality of analytes, wherein the one or more intermediate probes are detectable using one or more detectably-labeled probes, and dehybridizing the one or more intermediate probes and/or the one or more detectably-labeled probes from the nucleic acid sequences. In some instances, the contacting and dehybridizing steps are repeated with the one or more intermediate probes, the one or more detectably-labeled probes, one or more other intermediate probes, and/or one or more other detectably-labeled probes. In some embodiments, the one or more intermediate probes can each comprise a sequence that hybridizes to one of the nucleic acid sequences and one or more overhangs that hybridize to a detectably-labeled probe but not to the nucleic acid sequences.

In any of the preceding embodiments, detecting the first non-overlapping optical signal associated with the first analyte may comprise omitting a selected detectable probe targeting the second analyte and/or blocking the selected detectable probe from targeting the second analyte. In any of the preceding embodiments, detecting the second non-overlapping optical signal associated with the second analyte comprises omitting a selected detectable probe targeting the first analyte and/or blocking the selected detectable probe from targeting the first analyte. In any of the preceding embodiments, the blocking the detectable probe from targeting the second or first analyte comprises (i) directly or indirectly binding a probe to the detectable probe, thereby blocking the detectable probe from directly or indirectly binding to the second or first analyte, or (ii) directly or indirectly binding a probe to the second or first analyte, thereby blocking the second or first analyte from directly or indirectly binding to the detectable probe.

In any of the preceding embodiments, blocking the selected detectable probe from targeting the first or second analyte can comprise contacting the sample with an interfering agent, wherein the interfering agent interferes with hybridization of the selected detectable probe to its corresponding nucleic acid sequence in or associated with the corresponding analyte. In some embodiments, the selected detectable probe can be contacted with the interfering agent to form a detectable probe/interfering agent hybridization complex before the sample is contacted with the selected detectable probe. In some embodiments, the sample can be contacted with the interfering agent to form a hybridization complex between the interfering agent and the nucleic acid sequence in or associated with the corresponding analyte.

In any of the preceding embodiments, the method can comprise contacting the sample with a plurality of interfering agents. In any of the preceding embodiments, the interfering agent can displace the selected detectable probe which is hybridized to the nucleic acid sequence in or associated with the corresponding analyte.

In any of the preceding embodiments, the second cycle can comprise contacting the sample with an interfering agent that blocks hybridization and/or detection of the second analyte, and/or the third cycle can comprise contacting the sample with an interfering agent that blocks hybridization and/or detection of the first analyte.

In any of the preceding embodiments, the selected detectable probe can comprise one or more overhangs that do not hybridize the nucleic acid sequence in or associated with the corresponding analyte. In some embodiments, at least one of the one or more overhangs is capable of hybridizing to a detectably labelled detection probe. In some embodiments, the selected detectable probe comprises an overhang that is capable of hybridizing to a sequence of the interfering agent. In some embodiments, hybridization of the interfering agent to the overhang of the selected detectable probe can initiates strand displacement reaction whereby the interfering agent hybridizes to the selected detectable probe and displaces it from the corresponding nucleic acid sequence.

In any of the preceding embodiments, the interfering agent can hybridize to the nucleic acid sequence corresponding to the selected detectable probe and prevents, competes with, and/or displaces the selected detectable probe(s) from hybridizing to the nucleic acid sequence.

In any of the preceding embodiments, the interfering agent can be provided at a higher concentration than probe(s) for the target nucleic acid sequence.

In any of the preceding embodiments, the interfering agent can comprise a quencher moiety.

In some embodiments, the first cycle can be before or after the second cycle. In some instances, the first and second cycles are consecutive or separated by one or more other cycles.

In any of the preceding embodiments, the first cycle can be before or after the third cycle. In any of the preceding embodiments, the first and third cycles can be consecutive or separated by one or more other cycles.

In any of the preceding embodiments, the second cycle can be before or after the third cycle. In any of the preceding embodiments, the second and third cycles can be consecutive or separated by one or more other cycles.

In any of the preceding embodiments, in the second cycle, no optical signal associated with the second analyte may be detected at the location.

In any of the preceding embodiments, in the third cycle, no optical signal associated with the first analyte may be detected at the location.

In any of the preceding embodiments, the plurality of overlapping optical signals detected in the first cycle can be a first plurality of overlapping optical signals, and the detecting in step a) can further comprise: iv) in a fourth cycle, detecting a second plurality of overlapping optical signals at the location.

In any of the preceding embodiments, the sequential cycles can comprise a fifth cycle in which no optical signal associated with the first analyte and no optical signal associated with the second analyte is detected at the location.

In any of the preceding embodiments, in the fifth cycle, an optical signal associated with a third analyte can be detected at the location.

In any of the preceding embodiments, the plurality of optical signals can be associated with detectable probes targeting at least a subset of the plurality of analytes in each cycle.

In any of the preceding embodiments, the detectable probes each can comprise a fluorophore.

In any of the preceding embodiments, the detectable probes each can comprise an analyte targeting region.

In any of the preceding embodiments, two or more detectable probes targeting the same analyte can comprise the same analyte targeting region or different analyte targeting regions.

In any of the preceding embodiments, the detectable probes each can comprise a probe binding region that binds to a fluorescently labelled probe.

In any of the preceding embodiments, two or more detectable probes targeting the same analyte can comprise the same probe binding region or different probe binding regions.

In any of the preceding embodiments, two or more detectable probes targeting the same analyte can bind to the same fluorescently labelled probe or different fluorescently labelled probes.

In another aspect, also provided here in is a method for analyzing a biological sample comprising a plurality of analytes, comprising: (a) in sequential cycles, contacting the biological sample with a plurality of detectable probes each comprising an analyte targeting region, wherein in the sequential cycles, detectable probes targeting a particular analyte are contacted with the biological sample according to an order of signal codes in an identifier that identifies that particular analyte among the plurality of analytes, the signal codes corresponding to optical signals associated with the detectable probes, and wherein the plurality of detectable probes comprise a first probe set targeting a first analyte and a second probe set targeting a second analyte, and the sequential cycles comprise: (i) one or more non-sparse cycles in which the biological sample is contacted with a detectable probe of the first probe set and a detectable probe of the second detectable probe set; and (ii) one or more sparse cycles in which the biological sample is contacted with a detectable probe of the first probe set and no detectable probe of the second probe set, or vice versa; (b) in the sequential cycles, detecting optical signals associated with the detectable probes at a location in the biological sample to provide optical signatures comprising the detected optical signals for analytes at the location, wherein in a non-sparse cycle, optical signals associated with the first and second analytes are overlapping at the location, resulting in an ambiguity in analyte identity at the location, and wherein in a sparse cycle, the optical signal associated with the first analyte at the location does not overlap with an optical signal associated with the second analyte, or vice versa; and (c) comparing the optical signatures for analytes at the location to the identifiers for the plurality of analytes, thereby associating the overlapping optical signals at the location to the first analyte and the second analyte, respectively, to resolve the ambiguity in the non-sparse cycle, thereby identifying the first and second analytes at the location.

In yet another aspect, provided herein is a method for analyzing a biological sample comprising a plurality of analytes, comprising: (a) in sequential cycles, contacting the biological sample with a plurality of detectable probes each comprising an analyte targeting region and a fluorescently labelled probe binding region, wherein in the sequential cycles, detectable probes targeting a particular analyte are contacted with the biological sample according to an order of signal codes in an identifier that identifies that particular analyte among the plurality of analytes, the signal codes corresponding to optical signals associated with the fluorescently labelled probe binding regions of the detectable probes, and wherein the plurality of detectable probes comprise a first probe set targeting a first analyte and a second probe set targeting a second analyte, and the sequential cycles comprise: (i) one or more non-sparse cycles in which the biological sample is contacted with a detectable probe of the first probe set and a detectable probe of the second detectable probe set; and (ii) one or more sparse cycles in which the biological sample is contacted with a detectable probe of the first probe set and no detectable probe of the second probe set, or vice versa; (b) in the sequential cycles, contacting the biological sample with fluorescently labelled probes each capable of binding to the fluorescently labelled probe binding region of a detectable probe in that particular cycle; (c) detecting optical signals associated with the fluorescently labelled probes at a location in the biological sample to provide optical signatures comprising the detected optical signals for analytes at the location, wherein in a non-sparse cycle, optical signals associated with the first and second analytes are overlapping at the location, resulting in an ambiguity in analyte identity at the location, and wherein in a sparse cycle, the optical signal associated with the first analyte at the location does not overlap with an optical signal associated with the second analyte, or vice versa; and (c) comparing the optical signatures for analytes at the location to the identifiers for the plurality of analytes, thereby associating the overlapping optical signals at the location to the first analyte and the second analyte, respectively, to resolve the ambiguity in the non-sparse cycle, thereby identifying the first and second analytes at the location.

In any of the preceding embodiments, an optical signal corresponding to the sparse cycle can be removed from optical signatures for analytes at the location as a result of resolving the ambiguity.

In any of the preceding embodiments, the location can be a first location, and wherein at a second location in the biological sample, optical signals associated with the first and second analytes can be non-overlapping in the non-sparse cycle.

In any of the preceding embodiments, optical signals associated with the first and second analytes can be non-overlapping at the second location in the sparse cycle.

In any of the preceding embodiments, optical signatures at the second location can comprise optical signals of the non-sparse cycle and the sparse cycle.

In any of the preceding embodiments, at the second location, an optical signal associated with a detectable probe may not be detected, and the absence of optical signal is recorded as part of the optical signature.

In any of the preceding embodiments, the optical signature at the second location can be compared to the identifiers for the plurality of analytes.

In any of the preceding embodiments, the recorded absence of optical signal can be removed from the optical signature at the second location, and wherein the first or second analyte can be identified at the second location.

In any of the preceding embodiments, the non-sparse cycle can be before or after the sparse cycle.

In any of the preceding embodiments, the non-sparse cycle and the sparse cycle can be consecutive or separated by one or more other cycles.

In any of the preceding embodiments, in the non-sparse cycle, the biological sample can be contacted with a detectable probe targeting each of the plurality of analytes.

In any of the preceding embodiments, in the sparse cycle, the biological sample can be contacted with a detectable probe targeting each of the plurality of analytes except the first analyte or the second analyte.

In any of the preceding embodiments, the sequential cycles can comprise two, three, four, five, six, or more non-sparse cycles for the first and second analytes.

In any of the preceding embodiments, the sequential cycles can comprise two, three, four, five, six, or more sparse cycles for the first analyte and/or the second analyte.

In any of the preceding embodiments, two, three, four, five, six, or more of the detectable probes targeting the particular analyte can comprise different fluorescently labelled probe binding regions.

In any of the preceding embodiments, the plurality of detectable probes can comprise four different fluorescently labelled probe binding regions each capable of hybridizing to a fluorescently labelled probe comprising a different fluorophore.

In any of the preceding embodiments, the first probe set and the second probe set can comprise detectable probes comprising the same fluorescently labelled probe binding region.

In any of the preceding embodiments, the first probe set and the second probe set can comprise detectable probes comprising different fluorescently labelled probe binding regions.

In any of the preceding embodiments, the method can comprise three, four, five, six, or more sequential cycles.

In any of the preceding embodiments, the method can comprise identifying at least 10, at least 25, at least 50, at least 100, at least 250, at least 500, at least 1,000, at least 2,500, or more different analytes.

In any of the preceding embodiments, the detecting steps can be performed in situ in the biological sample.

In any of the preceding embodiments, the plurality of analytes can comprise nucleic acid analytes and/or protein analytes.

In any of the preceding embodiments, the nucleic acid analytes can comprise rolling circle amplification (RCA) products.

In any of the preceding embodiments, analytes at at least 10, at least 50, at least 100, at least 500, at least 1,000, at least 5,000, at least 10,000, or more different locations in the biological sample can be analyzed.

In another aspect, provided herein is a method for analyzing a biological sample comprising a plurality of analytes, comprising: (a) in sequential cycles, contacting the biological sample with a plurality of detectable probes, wherein the biological sample comprises multiple rolling circle amplification (RCA) products each comprising a target nucleic acid sequence corresponding to an analyte, wherein each detectable probe comprises (i) a target hybridizing region that hybridizes to the target nucleic acid sequence and (ii) a fluorescently labelled probe hybridizing region, wherein in the sequential cycles, detectable probes for a particular analyte are contacted with the biological sample according to an identifier comprising an order of signal codes that identifies that particular analyte among the plurality of analytes, the signal codes corresponding to optical signals from the fluorescently labelled probes that hybridize to the detectable probes, and wherein the plurality of detectable probes comprise a first probe set for a first analyte and a second probe set for a second analyte, and the sequential cycles comprise: (i) one or more non-sparse cycles in which the biological sample is contacted with a detectable probe of the first probe set and a detectable probe of the second detectable probe set; and (ii) one or more sparse cycles in which the biological sample is contacted with a detectable probe of the first probe set and no detectable probe of the second probe set, or vice versa; (b) in the sequential cycles, contacting the biological sample with fluorescently labelled probes each capable of hybridizing a detectable probe in that particular cycle; (c) detecting optical signals associated with the fluorescently labelled probes at a location in the biological sample to provide optical signatures comprising the detected optical signals for analytes at the location, wherein in a non-sparse cycle, optical signals associated with the first and second analytes are overlapping at the location, resulting in an ambiguity in analyte identity at the location, and wherein in a sparse cycle, the optical signal associated with the first analyte at the location does not overlap with an optical signal associated with the second analyte, or vice versa; and (d) comparing the optical signatures for analytes at the location to the identifiers for the plurality of analytes, thereby associating the overlapping optical signals at the location to the first analyte and the second analyte, respectively, to resolve the ambiguity in the non-sparse cycle, thereby identifying the first and second analytes at the location.

In some aspects, provided herein is a method for analyzing a biological sample comprising a plurality of analytes, comprising: in sequential cycles, contacting the biological sample with a plurality of detectable probes each comprising an analyte targeting region, wherein in the sequential cycles, detectable probes targeting a particular analyte are contacted with the biological sample according to an order of signal codes in an identifier that identifies that particular analyte among the plurality of analytes, the signal codes corresponding to signals associated with the detectable probes, and wherein the plurality of detectable probes comprise a first probe set targeting a first analyte and a second probe set targeting a second analyte, and the sequential cycles comprise one or more sparse cycles in which the biological sample is contacted with: (i) a detectable probe of the first probe set, a detectable probe of the second probe set, and an interfering agent that blocks hybridization and/or detection of the detectable probe of the second probe set, wherein a signal associated with a detectable probe of the first probe set is detected and a signal associated with a detectable probe of the second probe set is not detected, or (ii) a detectable probe of the first probe set, a detectable probe of the second probe set, and an interfering agent that blocks hybridization and/or detection of the detectable probe of the first probe set, wherein a signal associated with a detectable probe of the second probe set is detected and a signal associated with a detectable probe of the first probe set is not detected; thereby determining a sequential sequence of signal codes that identify the first analyte and the second analyte. In some embodiments, the sequential cycles comprise one or more non-sparse cycles in which the biological sample is contacted with a detectable probe of the first probe set and a detectable probe of the second detectable probe set in the absence of an interfering agent; and wherein in a non-sparse cycle, optical signals associated with the first and second analytes are overlapping at the location, resulting in an ambiguity in analyte identity at the location, and wherein in a sparse cycle, the optical signal associated with the first analyte at the location does not overlap with an optical signal associated with the second analyte, or vice versa; and (d) comparing the plurality of potential signal sequence chains for analytes at the location to the identifiers for the plurality of analytes to identify a match, thereby associating the overlapping optical signals at the location to the first analyte and the second analyte, respectively, thereby resolving the ambiguity in the non-sparse cycle, thereby identifying the first and second analytes at the location.

In some embodiments, the target nucleic acid sequence can be a barcode sequence or complement thereof.

In any of the preceding embodiments, each RCA product can comprise multiple copies of the barcode sequence or complement thereof.

In any of the preceding embodiments, at least three, four, five, six, or more of the plurality of detectable probes can comprise the same target hybridizing region but different fluorescently labelled probe hybridizing regions.

In any of the preceding embodiments, at least three, four, five, six, or more of the plurality of detectable probes can comprise different target hybridizing regions but the same fluorescently labelled probe hybridizing region.

In any of the preceding embodiments, the multiple RCA products can be generated and detected in situ.

In any of the preceding embodiments, the multiple RCA products can be generated using a circular or circularizable probe or probe set that hybridizes to: (i) a nucleic acid analyte in the biological sample; (ii) a product of a nucleic acid analyte in the biological sample; (iii) a reporter oligonucleotide of a labelling agent that directly or indirectly binds to a nucleic acid analyte or a non-nucleic acid analyte in the biological sample; or (iv) a product of a reporter oligonucleotide of a labelling agent that directly or indirectly binds to a nucleic acid analyte or a non-nucleic acid analyte in the biological sample.

In any of the preceding embodiments, the nucleic acid analyte can be an mRNA and/or the non-nucleic acid analyte can be a protein.

In any of the preceding embodiments, the biological sample can be a processed or cleared tissue sample.

In any of the preceding embodiments, the biological sample can be a tissue slice between about 1 μm and about 50 μm in thickness In any of the preceding embodiments, the tissue slice can be between about 5 μm and about 35 μm in thickness. In any of the preceding embodiments, the biological sample can be embedded in a hydrogel. In any of the preceding embodiments, the biological sample may not be embedded in a hydrogel. In any of the preceding embodiments, the biological sample can be cross-linked.

### BRIEF DESCRIPTION OF THE DRAWINGS

The drawings illustrate certain embodiments of the features and advantages of this disclosure. These embodiments are not intended to limit the scope of the appended claims in any manner.

This patent or application file contains at least one drawing executed in color. Copies of this patent or patent application publication with color drawing(s) will be provided by the Office upon request and payment of the necessary fee.

FIG. 1 shows a schematic illustrating typical problems which occur with molecular and optical crowding when using a regular combinatorial decoding scheme.

FIG. 2 shows a schematic illustrating the detection of overlapping signals using an exemplary sparse labelling scheme disclosed herein.

FIG. 3 shows a representative plot of the results of an exemplary sparse labelling detection method on a mouse brain tissue section.

FIG. 4 shows a schematic illustrating the detection of overlapping signals according to an exemplary sparse labelling scheme using interfering agents disclosed herein.

FIGS. 5A-5B depict exemplary interfering agent designs for displacing a selected probe (e.g., in a dark cycle).

FIGS. 6A-6B depict interfering agent designs comprising quencher moieties.

### DETAILED DESCRIPTION

All publications, comprising patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Overview

In multiplex assays, such as multiplexed in situ gene expression and/or protein analysis, signal crowding problems can arise when there are a large number of signals to be detected. For example, combinatorial barcoding approaches are often used to encode a large number of analytes, and optical signals (e.g., spots in fluorescent microscopy) from the analytes or probes bound thereto are detected and decoded. Because the number of spots detected typically scales with the number of analytes assayed, a sample can become crowded with signal spots that overlap with each other thereby making resolution of individual spots difficult and reducing overall assay sensitivity. Thus, spatial overlap may limit the ability to multiplex in assays such as microscopy-based nucleic acid hybridization or sequencing assays.

In some aspects, signal crowding may arise when one or more of the signals being detected are significantly stronger (e.g., have a significantly larger amplitude) than other signal(s). For example, in the same microscope field of view, one or more fluorescent spots may be significantly stronger than other spots including neighboring spots. In other aspects, signal crowding may arise when one or more of the signals being detected are in close proximity (e.g., overlapping to some degree) with other signal(s), such as overlapping signals observed in the same microscope field of view. When too many signals (e.g., “spots”) are present or when the amplitude of a signal is significantly greater than that of another signal, it can be difficult to accurately and reliably detect all of the signals in the same field of view and/or in the same detection channel (e.g., the same fluorescent channel). In some examples, this can cause weaker (e.g., lower amplitude) or overlapping signals to be “crowded out” or masked, which ultimately leads to information from the system being lost. In such circumstances, the effective dynamic range of the detection assay can be reduced.

The present inventors have developed methods of detecting multiple analytes (e.g., target nucleic acid sequences or proteins) in a sample so as to reduce signal crowding contributed by detection of the one or more analytes. In some embodiments, methods and compositions disclosed herein address issues associated with overlapping or very close signals that may be detected as one single signal using common optical detection method. In some embodiments, overlapping signals may need to be discarded as they cannot be used to accurately decode barcodes for analyte detection, and information associated with the overlapping signals will be lost. In some embodiments, methods and compositions disclosed herein enable the resolution of signals that would otherwise be overlapping into distinct signals that can be used to accurately decode barcodes for analyte detection.

In some embodiments, an analyte known to cause (or suspected of contributing to) signal crowding in a sample is selected prior to a given cycle, step, or round of a method disclosed herein, and a detection probe for the selected analyte, a secondary (or higher order) probe specific to a sequence in, e.g., the detection probe or an amplified product thereof, and/or a detection agent (e.g., a fluorescently labelled detection oligo) for the analyte or probe(s) may be manipulated in the cycle, step, or round. In some embodiments, a plurality of analytes known to cause or suspected of contributing to signal crowding are selected. In some embodiments, hybridization between the selected analyte(s), probe(s) thereto, and/or detection agent(s) for the analyte(s) or probe(s) is manipulated. In some embodiments, the presence/absence or amount of probe(s) to the selected analyte(s) and/or detection agent(s) for the analyte(s) or probe(s) is manipulated, using a method disclosed herein. For example, an analyte known to cause or suspected of contributing to signal crowding is a highly expressed or highly abundant analyte in the sample.

In some embodiments, one or more analytes to be detected, probe(s) thereto, and/or detection agent(s) for the analyte(s) or probe(s) are manipulated in a cycle, step, or round of the method, wherein such analytes are not pre-selected based on the knowledge or suspicion that they may contribute to signal crowding, but are designated to be manipulated in the given cycle, step, or round in a random and/or combinatorial manner (e.g., from among multiple analytes to be detected in a sample). The multiple analytes to be detected may be pre-selected, e.g., targeted for analysis in a multiplex assay, but the designation of one or more of these analytes to be manipulated in a given cycle, step, or round is random and/or as part of a combinatorial scheme. In some embodiments, a plurality of analytes are randomly and/or combinatorially designated for manipulation in a cycle, step, or round of the method. In some embodiments, the presence/absence or amount of probe(s) to the designated analyte(s) and/or detection agent(s) for the analyte(s) or probe(s) is manipulated, for example, by omitting probe(s) and/or detection agent(s) for the designated analyte(s) in one or more cycles, steps, or rounds of a method disclosed herein.

In some embodiments, signals from one or more analytes to be detected, probe(s) thereto, and/or detection agent(s) for the analyte(s) or probe(s) are modified. These signals may be prevented from being generated and/or detected, or may be detected but reduced in amplitude, e.g., through manipulation of the analyte/probe binding (e.g., hybridization) and/or the probe/detection agent binding (e.g., hybridization). These signals may be also be generated and/or detected over an increased time period, e.g., by distributing signals that can be generated and/or detected in one cycle, step, or round among a plurality of cycles, steps, or rounds. Different analytes may be detected at different rounds or cycles, steps, or rounds of the method, and this may be achieved in a number of ways. For example, by employing “dark” rounds or cycles, wherein a target analyte or probe thereto is not detected (“rendered” or “read”) in that round or cycle. In some embodiments, a method disclosed herein reduces the peak number of signals that are generated and/detected from the sample at a given time, or in a given cycle, step, or round, and therefore reduces signal crowding. In some examples, these aspects of the present disclosure are referred to as the “sparse labelling” approach. In some embodiments, a method disclosed herein reduces the strengths of signals indicative of certain analyte(s) that are generated and/detected from the sample at a given time, or in a given cycle, step, or round. These signals (e.g., indicative of highly expressed genes in a sample), if not reduced, may crowd out or mask signals indicative of other analyte(s). The present disclosure, allows for more of the signals to be resolved, and therefore more of the analytes (e.g., target nucleic acid sequences) to be detected in a sample.

### II. Samples, Analytes, and Target Sequences

### A. Samples

A sample disclosed herein can be or derived from any biological sample. Methods and compositions disclosed herein may be used for analyzing a biological sample, which may be obtained from a subject using any of a variety of techniques including, but not limited to, biopsy, surgery, and laser capture microscopy (LCM), and generally includes cells and/or other biological material from the subject. In addition to the subjects described above, a biological sample can be obtained from a prokaryote such as a bacterium, an archaea, a virus, or a viroid. A biological sample can also be obtained from non-mammalian organisms (e.g., a plant, an insect, an arachnid, a nematode, a fungus, or an amphibian). A biological sample can also be obtained from a eukaryote, such as a tissue sample, a patient derived organoid (PDO) or patient derived xenograft (PDX). A biological sample from an organism may comprise one or more other organisms or components therefrom. For example, a mammalian tissue section may comprise a prion, a viroid, a virus, a bacterium, a fungus, or components from other organisms, in addition to mammalian cells and non-cellular tissue components. Subjects from which biological samples can be obtained can be healthy or asymptomatic individuals, individuals that have or are suspected of having a disease (e.g., a patient with a disease such as cancer) or a pre-disposition to a disease, and/or individuals in need of therapy or suspected of needing therapy.

The biological sample can include any number of macromolecules, for example, cellular macromolecules and organelles (e.g., mitochondria and nuclei). The biological sample can be a nucleic acid sample and/or protein sample. The biological sample can be a carbohydrate sample or a lipid sample. The biological sample can be obtained as a tissue sample, such as a tissue section, biopsy, a core biopsy, needle aspirate, or fine needle aspirate. The sample can be a fluid sample, such as a blood sample, urine sample, or saliva sample. The sample can be a skin sample, a colon sample, a cheek swab, a histology sample, a histopathology sample, a plasma or serum sample, a tumor sample, living cells, cultured cells, a clinical sample such as, for example, whole blood or blood-derived products, blood cells, or cultured tissues or cells, including cell suspensions. In some embodiments, macromolecules (e.g., analytes) from a sample are immobilized on a surface. In some embodiments, analytes from a solution are immobilized on a surface. In some embodiments, the biological sample may comprise cells which are deposited on a surface.

Cell-free biological samples can include extracellular polynucleotides. Extracellular polynucleotides can be isolated from a bodily sample, e.g., blood, plasma, serum, urine, saliva, mucosal excretions, sputum, stool, and tears.

Biological samples can be derived from a homogeneous culture or population of the subjects or organisms mentioned herein or alternatively from a collection of several different organisms, for example, in a community or ecosystem.

Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells. Biological samples can also include fetal cells and immune cells.

Biological samples can include analytes (e.g., protein, RNA, and/or DNA) embedded in a 3D matrix. In some embodiments, amplicons (e.g., rolling circle amplification products) derived from or associated with analytes (e.g., protein, RNA, and/or DNA) can be embedded in a 3D matrix. In some embodiments, a 3D matrix may comprise a network of natural molecules and/or synthetic molecules that are chemically and/or enzymatically linked, e.g., by crosslinking. In some embodiments, a 3D matrix may comprise a synthetic polymer. In some embodiments, a 3D matrix comprises a hydrogel.

In some embodiments, a substrate herein can be any support that is insoluble in aqueous liquid and which allows for positioning of biological samples, analytes, features, and/or reagents (e.g., probes) on the support. In some embodiments, a biological sample can be attached to a substrate. Attachment of the biological sample can be irreversible or reversible, depending upon the nature of the sample and subsequent steps in the analytical method. In certain embodiments, the sample can be attached to the substrate reversibly by applying a suitable polymer coating to the substrate, and contacting the sample to the polymer coating. The sample can then be detached from the substrate, e.g., using an organic solvent that at least partially dissolves the polymer coating. Hydrogels are examples of polymers that are suitable for this purpose.

In some embodiments, the substrate can be coated or functionalized with one or more substances to facilitate attachment of the sample to the substrate. Suitable substances that can be used to coat or functionalize the substrate include, but are not limited to, lectins, poly-lysine, antibodies, and polysaccharides.

A variety of steps can be performed to prepare or process a biological sample for and/or during an assay. Except where indicated otherwise, the preparative or processing steps described below can generally be combined in any manner and in any order to appropriately prepare or process a particular sample for and/or analysis.

### (i) Tissue Sectioning

A biological sample can be harvested from a subject (e.g., via surgical biopsy, whole subject sectioning) or grown in vitro on a growth substrate or culture dish as a population of cells, and prepared for analysis as a tissue slice or tissue section. Grown samples may be sufficiently thin for analysis without further processing steps. Alternatively, grown samples, and samples obtained via biopsy or sectioning, can be prepared as thin tissue sections using a mechanical cutting apparatus such as a vibrating blade microtome. As another alternative, in some embodiments, a thin tissue section can be prepared by applying a touch imprint of a biological sample to a suitable substrate material.

The thickness of the tissue section can be a fraction of (e.g., less than 0.9, 0.8, 0.7, 0.6, 0.5, 0.4, 0.3, 0.2, or 0.1) the maximum cross-sectional dimension of a cell. However, tissue sections having a thickness that is larger than the maximum cross-section cell dimension can also be used. For example, cryostat sections can be used, which can be, e.g., 10-20 μm thick.

More generally, the thickness of a tissue section typically depends on the method used to prepare the section and the physical characteristics of the tissue, and therefore sections having a wide variety of different thicknesses can be prepared and used. For example, the thickness of the tissue section can be at least 0.1, 0.2, 0.3, 0.4, 0.5, 0.7, 1.0, 1.5, 2, 3, 4, 5, 6, 7, 8, 9, 10, 12, 13, 14, 15, 20, 30, 40, or 50 μm. Thicker sections can also be used if desired or convenient, e.g., at least 70, 80, 90, or 100 μm or more. Typically, the thickness of a tissue section is between 1-100 μm, 1-50 μm, 1-30 μm, 1-25 μm, 1-20 μm, 1-15 μm, 1-10 μm, 2-8 μm, 3-7 μm, or 4-6 μm, but as mentioned above, sections with thicknesses larger or smaller than these ranges can also be analysed.

Multiple sections can also be obtained from a single biological sample. For example, multiple tissue sections can be obtained from a surgical biopsy sample by performing serial sectioning of the biopsy sample using a sectioning blade. Spatial information among the serial sections can be preserved in this manner, and the sections can be analysed successively to obtain three-dimensional information about the biological sample.

### (ii) Freezing

In some embodiments, the biological sample (e.g., a tissue section as described above) can be prepared by deep freezing at a temperature suitable to maintain or preserve the integrity (e.g., the physical characteristics) of the tissue structure. The frozen tissue sample can be sectioned, e.g., thinly sliced, onto a substrate surface using any number of suitable methods. For example, a tissue sample can be prepared using a chilled microtome (e.g., a cryostat) set at a temperature suitable to maintain both the structural integrity of the tissue sample and the chemical properties of the nucleic acids in the sample. Such a temperature can be, e.g., less than −15° C., less than −20° C., or less than −25° C.

(iii) Fixation and Postfixation

In some embodiments, the biological sample can be prepared using formalin-fixation and paraffin-embedding (FFPE), which are established methods. In some embodiments, cell suspensions and other non-tissue samples can be prepared using formalin-fixation and paraffin-embedding. Following fixation of the sample and embedding in a paraffin or resin block, the sample can be sectioned as described above. Prior to analysis, the paraffin-embedding material can be removed from the tissue section (e.g., deparaffinization) by incubating the tissue section in an appropriate solvent (e.g., xylene) followed by a rinse (e.g., 99.5% ethanol for 2 minutes, 96% ethanol for 2 minutes, and 70% ethanol for 2 minutes).

As an alternative to formalin fixation described above, a biological sample can be fixed in any of a variety of other fixatives to preserve the biological structure of the sample prior to analysis. For example, a sample can be fixed via immersion in ethanol, methanol, acetone, paraformaldehyde (PFA)-Triton, and combinations thereof.

In some embodiments, acetone fixation is used with fresh frozen samples, which can include, but are not limited to, cortex tissue, mouse olfactory bulb, human brain tumor, human post-mortem brain, and breast cancer samples. When acetone fixation is performed, pre-permeabilization steps (described below) may not be performed. Alternatively, acetone fixation can be performed in conjunction with permeabilization steps.

In some embodiments, the methods provided herein comprises one or more post-fixing (also referred to as postfixation) steps. In some embodiments, one or more post-fixing step is performed after contacting a sample with a polynucleotide disclosed herein, e.g., one or more probes such as a circular or padlock probe. In some embodiments, one or more post-fixing step is performed after a hybridization complex comprising a probe and a target is formed in a sample. In some embodiments, one or more post-fixing step is performed prior to a ligation reaction disclosed herein, such as the ligation to circularize a padlock probe.

In some embodiments, one or more post-fixing step is performed after contacting a sample with a binding or labelling agent (e.g., an antibody or antigen binding fragment thereof) for a non-nucleic acid analyte such as a protein analyte. The labelling agent can comprise a nucleic acid molecule (e.g., reporter oligonucleotide) comprising a sequence corresponding to the labelling agent and therefore corresponds to (e.g., uniquely identifies) the analyte. In some embodiments, the labelling agent can comprise a reporter oligonucleotide comprising one or more barcode sequences.

A post-fixing step may be performed using any suitable fixation reagent disclosed herein, for example, 3% (w/v) paraformaldehyde in DEPC-PBS.

### (iv) Embedding

As an alternative to paraffin embedding described above, a biological sample can be embedded in any of a variety of other embedding materials to provide structural substrate to the sample prior to sectioning and other handling steps. In some cases, the embedding material can be removed e.g., prior to analysis of tissue sections obtained from the sample. Suitable embedding materials include, but are not limited to, waxes, resins (e.g., methacrylate resins), epoxies, and agar.

In some embodiments, the biological sample can be embedded in a matrix (e.g., a hydrogel matrix). Embedding the sample in this manner typically involves contacting the biological sample with a hydrogel such that the biological sample becomes surrounded by the hydrogel. For example, the sample can be embedded by contacting the sample with a suitable polymer material, and activating the polymer material to form a hydrogel. In some embodiments, the hydrogel is formed such that the hydrogel is internalized within the biological sample.

In some embodiments, the biological sample is immobilized in the hydrogel via cross-linking of the polymer material that forms the hydrogel. Cross-linking can be performed chemically and/or photochemically, or alternatively by any other suitable hydrogel-formation method.

The composition and application of the hydrogel-matrix to a biological sample typically depends on the nature and preparation of the biological sample (e.g., sectioned, non-sectioned, type of fixation). As one example, where the biological sample is a tissue section, the hydrogel-matrix can include a monomer solution and an ammonium persulfate (APS) initiator/tetramethylethylenediamine (TEMED) accelerator solution. As another example, where the biological sample consists of cells (e.g., cultured cells or cells disassociated from a tissue sample), the cells can be incubated with the monomer solution and APS/TEMED solutions. For cells, hydrogel-matrix gels are formed in compartments, including but not limited to devices used to culture, maintain, or transport the cells. For example, hydrogel-matrices can be formed with monomer solution plus APS/TEMED added to the compartment to a depth ranging from about 0.1 μm to about 2 mm.

Additional methods and aspects of hydrogel embedding of biological samples are described for example in Chen et al., Science 347(6221):543-548, 2015, the entire contents of which are incorporated herein by reference.

### (v) Staining and Immunohistochemistry

To facilitate visualization, biological samples can be stained using a wide variety of stains and staining techniques. In some embodiments, for example, a sample can be stained using any number of stains and/or immunohistochemical reagents. One or more staining steps may be performed to prepare or process a biological sample for an assay described herein or may be performed during and/or after an assay. In some embodiments, the sample can be contacted with one or more nucleic acid stains, membrane stains (e.g., cellular or nuclear membrane), cytological stains, or combinations thereof. In some examples, the stain may be specific to proteins, phospholipids, DNA (e.g., dsDNA, ssDNA), RNA, an organelle or compartment of the cell. The sample may be contacted with one or more labeled antibodies (e.g., a primary antibody specific for the analyte of interest and a labeled secondary antibody specific for the primary antibody). In some embodiments, cells in the sample can be segmented using one or more images taken of the stained sample.

In some embodiments, the stain is performed using a lipophilic dye. In some examples, the staining is performed with a lipophilic carbocyanine or aminostyryl dye, or analogs thereof (e.g, DiI, DiO, DiR, DiD). Other cell membrane stains may include FM and RH dyes or immunohistochemical reagents specific for cell membrane proteins. In some examples, the stain may include but is not limited to, acridine orange, acid fuchsin, Bismarck brown, carmine, coomassie blue, cresyl violet, DAPI, eosin, ethidium bromide, acid fuchsine, haematoxylin, Hoechst stains, iodine, methyl green, methylene blue, neutral red, Nile blue, Nile red, osmium tetroxide, ruthenium red, propidium iodide, rhodamine (e.g., rhodamine B), or safranine, or derivatives thereof. In some embodiments, the sample may be stained with haematoxylin and eosin (H&E).

The sample can be stained using hematoxylin and eosin (H&E) staining techniques, using Papanicolaou staining techniques, Masson's trichrome staining techniques, silver staining techniques, Sudan staining techniques, and/or using Periodic Acid Schiff (PAS) staining techniques. PAS staining is typically performed after formalin or acetone fixation. In some embodiments, the sample can be stained using Romanowsky stain, including Wright's stain, Jenner's stain, Can-Grunwald stain, Leishman stain, and Giemsa stain.

In some embodiments, biological samples can be destained. Methods of destaining or discoloring a biological sample generally depend on the nature of the stain(s) applied to the sample. For example, in some embodiments, one or more immunofluorescent stains are applied to the sample via antibody coupling. Such stains can be removed using techniques such as cleavage of disulfide linkages via treatment with a reducing agent and detergent washing, chaotropic salt treatment, treatment with antigen retrieval solution, and treatment with an acidic glycine buffer. Methods for multiplexed staining and destaining are described, for example, in Bolognesi et al., J. Histochem. Cytochem. 2017; 65(8): 431-444, Lin et al., Nat Commun. 2015; 6:8390, Pirici et al., J. Histochem. Cytochem. 2009; 57:567-75, and Glass et al., J. Histochem. Cytochem. 2009; 57:899-905, the entire contents of each of which are incorporated herein by reference.

### (vi) Isometric Expansion

In some embodiments, a biological sample embedded in a matrix (e.g., a hydrogel) can be isometrically expanded. Isometric expansion methods that can be used include hydration, a preparative step in expansion microscopy, as described in, e.g., Chen et al., Science 347(6221):543-548, 2015 and U.S. Pat. No. 10,059,990, which are herein incorporated by reference in their entireties.

Isometric expansion can be performed by anchoring one or more components of a biological sample to a gel, followed by gel formation, proteolysis, and swelling. In some embodiments, analytes in the sample, products of the analytes, and/or probes associated with analytes in the sample can be anchored to the matrix (e.g., hydrogel). Isometric expansion of the biological sample can occur prior to immobilization of the biological sample on a substrate, or after the biological sample is immobilized to a substrate. In some embodiments, the isometrically expanded biological sample can be removed from the substrate prior to contacting the substrate with probes disclosed herein.

In general, the steps used to perform isometric expansion of the biological sample can depend on the characteristics of the sample (e.g., thickness of tissue section, fixation, cross-linking), and/or the analyte of interest (e.g., different conditions to anchor RNA, DNA, and protein to a gel).

In some embodiments, proteins in the biological sample are anchored to a swellable gel such as a polyelectrolyte gel. An antibody can be directed to the protein before, after, or in conjunction with being anchored to the swellable gel. DNA and/or RNA in a biological sample can also be anchored to the swellable gel via a suitable linker. Examples of such linkers include, but are not limited to, 6-((Acryloyl)amino) hexanoic acid (Acryloyl-X SE) (available from ThermoFisher, Waltham, Mass.), Label-IT Amine (available from MirusBio, Madison, Wis.) and Label X (described for example in Chen et al., Nat. Methods 13:679-684, 2016, the entire contents of which are incorporated herein by reference).

Isometric expansion of the sample can increase the spatial resolution of the subsequent analysis of the sample. The increased resolution in spatial profiling can be determined by comparison of an isometrically expanded sample with a sample that has not been isometrically expanded.

In some embodiments, a biological sample is isometrically expanded to a size at least 2×, 2.1×, 2.2×, 2.3×, 2.4×, 2.5×, 2.6×, 2.7×, 2.8×, 2.9×, 3×, 3.1×, 3.2×, 3.3×, 3.4×, 3.5×, 3.6×, 3.7×, 3.8×, 3.9×, 4×, 4.1×, 4.2×, 4.3×, 4.4×, 4.5×, 4.6×, 4.7×, 4.8×, or 4.9× its non-expanded size. In some embodiments, the sample is isometrically expanded to at least 2× and less than 20× of its non-expanded size.

(vii) Crosslinking and De-Crosslinking

In some embodiments, the biological sample is reversibly cross-linked prior to or during an in situ assay round. In some aspects, the analytes, polynucleotides and/or amplification product (e.g., amplicon) of an analyte or a probe bound thereto can be anchored to a polymer matrix. For example, the polymer matrix can be a hydrogel. In some embodiments, one or more of the polynucleotide probe(s) and/or amplification product (e.g., amplicon) thereof can be modified to contain functional groups that can be used as an anchoring site to attach the polynucleotide probes and/or amplification product to a polymer matrix. In some embodiments, a modified probe comprising oligo dT may be used to bind to mRNA molecules of interest, followed by reversible or irreversible crosslinking of the mRNA molecules.

In some embodiments, the biological sample is immobilized in a hydrogel via cross-linking of the polymer material that forms the hydrogel. Cross-linking can be performed chemically and/or photochemically, or alternatively by any other suitable hydrogel-formation method. A hydrogel may include a macromolecular polymer gel including a network. Within the network, some polymer chains can optionally be cross-linked, although cross-linking does not always occur.

In some embodiments, a hydrogel can include hydrogel subunits, such as, but not limited to, acrylamide, bis-acrylamide, polyacrylamide and derivatives thereof, poly(ethylene glycol) and derivatives thereof (e.g. PEG-acrylate (PEG-DA), PEG-RGD), gelatin-methacryloyl (GelMA), methacrylated hyaluronic acid (MeHA), polyaliphatic polyurethanes, polyether polyurethanes, polyester polyurethanes, polyethylene copolymers, polyamides, polyvinyl alcohols, polypropylene glycol, polytetramethylene oxide, polyvinyl pyrrolidone, polyacrylamide, poly(hydroxyethyl acrylate), and poly(hydroxyethyl methacrylate), collagen, hyaluronic acid, chitosan, dextran, agarose, gelatin, alginate, protein polymers, methylcellulose, and the like, and combinations thereof.

In some embodiments, a hydrogel includes a hybrid material, e.g., the hydrogel material includes elements of both synthetic and natural polymers. Examples of suitable hydrogels are described, for example, in U.S. Pat. Nos. 6,391,937, 9,512,422, and 9,889,422, and in U.S. Patent Application Publication Nos. 2017/0253918, 2018/0052081 and 2010/0055733, the entire contents of each of which are incorporated herein by reference.

In some embodiments, the hydrogel can form the substrate. In some embodiments, the substrate includes a hydrogel and one or more second materials. In some embodiments, the hydrogel is placed on top of one or more second materials. For example, the hydrogel can be pre-formed and then placed on top of, underneath, or in any other configuration with one or more second materials. In some embodiments, hydrogel formation occurs after contacting one or more second materials during formation of the substrate. Hydrogel formation can also occur within a structure (e.g., wells, ridges, projections, and/or markings) located on a substrate.

In some embodiments, hydrogel formation on a substrate occurs before, contemporaneously with, or after probes are provided to the sample. For example, hydrogel formation can be performed on the substrate already containing the probes.

In some embodiments, hydrogel formation occurs within a biological sample. In some embodiments, a biological sample (e.g., tissue section) is embedded in a hydrogel. In some embodiments, hydrogel subunits are infused into the biological sample, and polymerization of the hydrogel is initiated by an external or internal stimulus.

In embodiments in which a hydrogel is formed within a biological sample, functionalization chemistry can be used. In some embodiments, functionalization chemistry includes hydrogel-tissue chemistry (HTC). Any hydrogel-tissue backbone (e.g., synthetic or native) suitable for HTC can be used for anchoring biological macromolecules and modulating functionalization. Non-limiting examples of methods using HTC backbone variants include CLARITY, PACT, ExM, SWITCH and ePACT. In some embodiments, hydrogel formation within a biological sample is permanent. For example, biological macromolecules can permanently adhere to the hydrogel allowing multiple rounds of interrogation. In some embodiments, hydrogel formation within a biological sample is reversible.

In some embodiments, additional reagents are added to the hydrogel subunits before, contemporaneously with, and/or after polymerization. For example, additional reagents can include but are not limited to oligonucleotides (e.g., probes), endonucleases to fragment DNA, fragmentation buffer for DNA, DNA polymerase enzymes, dNTPs used to amplify the nucleic acid and to attach the barcode to the amplified fragments. Other enzymes can be used, including without limitation, RNA polymerase, transposase, ligase, proteinase K, and DNAse. Additional reagents can also include reverse transcriptase enzymes, including enzymes with terminal transferase activity, primers, and switch oligonucleotides. In some embodiments, optical labels are added to the hydrogel subunits before, contemporaneously with, and/or after polymerization.

In some embodiments, HTC reagents are added to the hydrogel before, contemporaneously with, and/or after polymerization. In some embodiments, a cell labelling agent is added to the hydrogel before, contemporaneously with, and/or after polymerization. In some embodiments, a cell-penetrating agent is added to the hydrogel before, contemporaneously with, and/or after polymerization.

Hydrogels embedded within biological samples can be cleared using any suitable method. For example, electrophoretic tissue clearing methods can be used to remove biological macromolecules from the hydrogel-embedded sample. In some embodiments, a hydrogel-embedded sample is stored before or after clearing of hydrogel, in a medium (e.g., a mounting medium, methylcellulose, or other semi-solid mediums). In some embodiments, a method disclosed herein comprises de-crosslinking the reversibly cross-linked biological sample.

(viii) Tissue Permeabilization and Treatment

In some embodiments, a biological sample can be permeabilized to facilitate transfer of species (such as probes) into the sample. If a sample is not permeabilized sufficiently, the transfer of species (such as probes) into the sample may be too low to enable adequate analysis. Conversely, if the tissue sample is too permeable, the relative spatial relationship of the analytes within the tissue sample can be lost. Hence, a balance between permeabilizing the tissue sample enough to obtain good signal intensity while still maintaining the spatial resolution of the analyte distribution in the sample is desirable.

In general, a biological sample can be permeabilized by exposing the sample to one or more permeabilizing agents. Suitable agents for this purpose include, but are not limited to, organic solvents (e.g., acetone, ethanol, and methanol), cross-linking agents (e.g., paraformaldehyde), detergents (e.g., saponin, Triton X-100™ or Tween-20™), and enzymes (e.g., trypsin, proteases). In some embodiments, the biological sample can be incubated with a cellular permeabilizing agent to facilitate permeabilization of the sample. Additional methods for sample permeabilization are described, for example, in Jamur et al., Method Mol. Biol. 588:63-66, 2010, the entire contents of which are incorporated herein by reference. Any suitable method for sample permeabilization can generally be used in connection with the samples described herein.

In some embodiments, the biological sample can be permeabilized by adding one or more lysis reagents to the sample. Examples of suitable lysis agents include, but are not limited to, bioactive reagents such as lysis enzymes that are used for lysis of different cell types, e.g., gram positive or negative bacteria, plants, yeast, mammalian, such as lysozymes, achromopeptidase, lysostaphin, labiase, kitalase, lyticase, and a variety of other commercially available lysis enzymes.

Other lysis agents can additionally or alternatively be added to the biological sample to facilitate permeabilization. For example, surfactant-based lysis solutions can be used to lyse sample cells. Lysis solutions can include ionic surfactants such as, for example, sarcosyl and sodium dodecyl sulfate (SDS). More generally, chemical lysis agents can include, without limitation, organic solvents, chelating agents, detergents, surfactants, and chaotropic agents.

In some embodiments, the biological sample can be permeabilized by non-chemical permeabilization methods. For example, non-chemical permeabilization methods that can be used include, but are not limited to, physical lysis techniques such as electroporation, mechanical permeabilization methods (e.g., bead beating using a homogenizer and grinding balls to mechanically disrupt sample tissue structures), acoustic permeabilization (e.g., sonication), and thermal lysis techniques such as heating to induce thermal permeabilization of the sample.

Additional reagents can be added to a biological sample to perform various functions prior to analysis of the sample. In some embodiments, DNase and RNase inactivating agents or inhibitors such as proteinase K, and/or chelating agents such as EDTA, can be added to the sample. For example, a method disclosed herein may comprise a step for increasing accessibility of a nucleic acid for binding, e.g., a denaturation step to opening up DNA in a cell for hybridization by a probe. For example, proteinase K treatment may be used to free up DNA with proteins bound thereto.

### (ix) Selective Enrichment of RNA Species

In some embodiments, where RNA is the analyte, one or more RNA analyte species of interest can be selectively enriched. For example, one or more species of RNA of interest can be selected by addition of one or more oligonucleotides to the sample. In some embodiments, the additional oligonucleotide is a sequence used for priming a reaction by an enzyme (e.g., a polymerase). For example, one or more primer sequences with sequence complementarity to one or more RNAs of interest can be used to amplify the one or more RNAs of interest, e.g., to generate cDNA, thereby selectively enriching these RNAs.

Alternatively, one or more species of RNA can be down-selected (e.g., removed) using any of a variety of methods. For example, probes can be administered to a sample that selectively hybridize to ribosomal RNA (rRNA), thereby reducing the pool and concentration of rRNA in the sample. Additionally and alternatively, duplex-specific nuclease (DSN) treatment can remove rRNA (see, e.g., Archer, et al, Selective and flexible depletion of problematic sequences from RNA-seq libraries at the cDNA stage, BMC Genomics, 15 401, (2014), the entire contents of which are incorporated herein by reference). Furthermore, hydroxyapatite chromatography can remove abundant species (e.g., rRNA) (see, e.g., Vandernoot, V. A., cDNA normalization by hydroxyapatite chromatography to enrich transcriptome diversity in RNA-seq applications, Biotechniques, 53(6) 373-80, (2012), the entire contents of which are incorporated herein by reference).

A biological sample may comprise one or a plurality of analytes of interest. Methods for performing multiplexed assays to analyze two or more different analytes in a single biological sample are provided.

### B. Analytes

The methods and compositions disclosed herein can be used to detect and analyze a wide variety of different analytes. In some aspects, an analyte can include any biological substance, structure, moiety, or component to be analyzed. In some aspects, a target disclosed herein may similarly include any analyte of interest. In some examples, a target or analyte can be directly or indirectly detected.

Analytes can be derived from a specific type of cell and/or a specific sub-cellular region. For example, analytes can be derived from cytosol, from cell nuclei, from mitochondria, from microsomes, and more generally, from any other compartment, organelle, or portion of a cell. Permeabilizing agents that specifically target certain cell compartments and organelles can be used to selectively release analytes from cells for analysis, and/or allow access of one or more reagents (e.g., probes for analyte detection) to the analytes in the cell or cell compartment or organelle.

The analyte may include any biomolecule or chemical compound, including a macromolecule such as a protein or peptide, a lipid or a nucleic acid molecule, or a small molecule, including organic or inorganic molecules. The analyte may be a cell or a microorganism, including a virus, or a fragment or product thereof. An analyte can be any substance or entity for which a specific binding partner (e.g. an affinity binding partner or labelling agent) can be developed. Such a specific binding partner may be a nucleic acid probe (for a nucleic acid analyte) and may lead directly to the generation of a rolling circle amplification (RCA) template (e.g. a padlock or other circularizable probe). Alternatively, the specific binding partner may be coupled to a nucleic acid (e.g., reporter oligonucleotide), which may be detected using an RCA strategy, e.g. in an assay which uses or generates a circular nucleic acid molecule which can be the RCA template.

Analytes of particular interest may include nucleic acid molecules, such as DNA (e.g. genomic DNA, mitochondrial DNA, plastid DNA, viral DNA, etc.) and RNA (e.g. mRNA, microRNA, rRNA, snRNA, viral RNA, etc.), and synthetic and/or modified nucleic acid molecules, (e.g. including nucleic acid domains comprising or consisting of synthetic or modified nucleotides such as LNA, PNA, morpholino, etc.), proteinaceous molecules such as peptides, polypeptides, proteins or prions or any molecule which includes a protein or polypeptide component, etc., or fragments thereof, or a lipid or carbohydrate molecule, or any molecule which comprise a lipid or carbohydrate component. The analyte may be a single molecule or a complex that contains two or more molecular subunits, e.g. including but not limited to protein-DNA complexes, which may or may not be covalently bound to one another, and which may be the same or different. Thus in addition to cells or microorganisms, such a complex analyte may also be a protein complex or protein interaction. Such a complex or interaction may thus be a homo- or hetero-multimer. Aggregates of molecules, e.g. proteins may also be target analytes, for example aggregates of the same protein or different proteins. The analyte may also be a complex between proteins or peptides and nucleic acid molecules such as DNA or RNA, e.g. interactions between proteins and nucleic acids, e.g. regulatory factors, such as transcription factors, and DNA or RNA.

(i) Endogenous Analytes

In some embodiments, an analyte herein is endogenous to a biological sample and can include nucleic acid analytes and non-nucleic acid analytes. Methods and compositions for reducing the signals detected in a given detection step, round, or cycle, of decoding disclosed herein (e.g., in Section IV) can be used to analyze nucleic acid analytes (e.g., using a nucleic acid probe or probe set that directly or indirectly hybridizes to a nucleic acid analyte) and/or non-nucleic acid analytes (e.g., using a labelling agent that comprises a reporter oligonucleotide and binds directly or indirectly to a non-nucleic acid analyte) in any suitable combination.

Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral coat proteins, extracellular and intracellular proteins, antibodies, and antigen binding fragments. In some embodiments, the analyte is inside a cell or on a cell surface, such as a transmembrane analyte or one that is attached to the cell membrane. In some embodiments, the analyte can be an organelle (e.g., nuclei or mitochondria). In some embodiments, the analyte is an extracellular analyte, such as a secreted analyte. Exemplary analytes include, but are not limited to, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, an extracellular matrix protein, a posttranslational modification (e.g., phosphorylation, glycosylation, ubiquitination, nitrosylation, methylation, acetylation or lipidation) state of a cell surface protein, a gap junction, and an adherens junction.

Examples of nucleic acid analytes include DNA analytes such as single-stranded DNA (ssDNA), double-stranded DNA (dsDNA), genomic DNA, methylated DNA, specific methylated DNA sequences, fragmented DNA, mitochondrial DNA, in situ synthesized PCR products, and RNA/DNA hybrids. The DNA analyte can be a transcript of another nucleic acid molecule (e.g., DNA or RNA such as mRNA) present in a tissue sample.

Examples of nucleic acid analytes also include RNA analytes such as various types of coding and non-coding RNA. Examples of the different types of RNA analytes include messenger RNA (mRNA), including a nascent RNA, a pre-mRNA, a primary-transcript RNA, and a processed RNA, such as a capped mRNA (e.g., with a 5′ 7-methyl guanosine cap), a polyadenylated mRNA (poly-A tail at the 3′ end), and a spliced mRNA in which one or more introns have been removed. Also included in the analytes disclosed herein are non-capped mRNA, a non-polyadenylated mRNA, and a non-spliced mRNA. The RNA analyte can be a transcript of another nucleic acid molecule (e.g., DNA or RNA such as viral RNA) present in a tissue sample. Examples of a non-coding RNAs (ncRNA) that is not translated into a protein include transfer RNAs (tRNAs) and ribosomal RNAs (rRNAs), as well as small non-coding RNAs such as microRNA (miRNA), small interfering RNA (siRNA), Piwi-interacting RNA (piRNA), small nucleolar RNA (snoRNA), small nuclear RNA (snRNA), extracellular RNA (exRNA), small Cajal body-specific RNAs (scaRNAs), and the long ncRNAs such as Xist and HOTAIR. The RNA can be small (e.g., less than 200 nucleic acid bases in length) or large (e.g., RNA greater than 200 nucleic acid bases in length). Examples of small RNAs include 5.8S ribosomal RNA (rRNA), 5S rRNA, tRNA, miRNA, siRNA, snoRNAs, piRNA, tRNA-derived small RNA (tsRNA), and small rDNA-derived RNA (srRNA). The RNA can be double-stranded RNA or single-stranded RNA. The RNA can be circular RNA. The RNA can be a bacterial rRNA (e.g., 16s rRNA or 23s rRNA).

In some embodiments described herein, an analyte may be a denatured nucleic acid, wherein the resulting denatured nucleic acid is single-stranded. The nucleic acid may be denatured, for example, optionally using formamide, heat, or both formamide and heat. In some embodiments, the nucleic acid is not denatured for use in a method disclosed herein.

In certain embodiments, an analyte can be extracted from a live cell. Processing conditions can be adjusted to ensure that a biological sample remains live during analysis, and analytes are extracted from (or released from) live cells of the sample. Live cell-derived analytes can be obtained only once from the sample, or can be obtained at intervals from a sample that continues to remain in viable condition.

Methods and compositions disclosed herein can be used to analyze any number of analytes. For example, the number of analytes that are analyzed can be at least about 2, at least about 3, at least about 4, at least about 5, at least about 6, at least about 7, at least about 8, at least about 9, at least about 10, at least about 11, at least about 12, at least about 13, at least about 14, at least about 15, at least about 20, at least about 25, at least about 30, at least about 40, at least about 50, at least about 100, at least about 1,000, at least about 10,000, at least about 100,000 or more different analytes present in a region of the sample or within an individual feature of the substrate.

In any embodiment described herein, the analyte comprises a target sequence. In some embodiments, the target sequence may be endogenous to the sample, generated in the sample, added to the sample, or associated with an analyte in the sample. In some embodiments, the target sequence is a single-stranded target sequence (e.g., a sequence in a rolling circle amplification product). In some embodiments, the analytes comprise one or more single-stranded target sequences. In one aspect, a first single-stranded target sequence is not identical to a second single-stranded target sequence. In another aspect, a first single-stranded target sequence is identical to one or more second single-stranded target sequence. In some embodiments, the one or more second single-stranded target sequence is comprised in the same analyte (e.g., nucleic acid) as the first single-stranded target sequence. Alternatively, the one or more second single-stranded target sequence is comprised in a different analyte (e.g., nucleic acid) from the first single-stranded target sequence.

(ii) Labelling Agents

In some embodiments, provided herein are methods and compositions for analyzing endogenous analytes (e.g., RNA, ssDNA, and cell surface or intracellular proteins and/or metabolites) in a sample using one or more labelling agents. In some embodiments, an analyte labelling agent may include an agent that interacts with an analyte (e.g., an endogenous analyte in a sample). In some embodiments, the labelling agents can comprise a reporter oligonucleotide that is indicative of the analyte or portion thereof interacting with the labelling agent. For example, the reporter oligonucleotide may comprise a barcode sequence that permits identification of the labelling agent. In some cases, the sample contacted by the labelling agent can be further contacted with a probe (e.g., a single-stranded probe sequence), that hybridizes to a reporter oligonucleotide of the labelling agent, in order to identify the analyte associated with the labelling agent. In some embodiments, the analyte labelling agent comprises an analyte binding moiety and a labelling agent barcode domain comprising one or more barcode sequences, e.g., a barcode sequence that corresponds to the analyte binding moiety and/or the analyte. An analyte binding moiety barcode includes to a barcode that is associated with or otherwise identifies the analyte binding moiety. In some embodiments, by identifying an analyte binding moiety by identifying its associated analyte binding moiety barcode, the analyte to which the analyte binding moiety binds can also be identified. An analyte binding moiety barcode can be a nucleic acid sequence of a given length and/or sequence that is associated with the analyte binding moiety. An analyte binding moiety barcode can generally include any of the variety of aspects of barcodes described herein.

In some embodiments, the method comprises one or more post-fixing (also referred to as post-fixation) steps after contacting the sample with one or more labelling agents.

In the methods and systems described herein, one or more labelling agents capable of binding to or otherwise coupling to one or more features may be used to characterize analytes, cells and/or cell features. In some instances, cell features include cell surface features. Analytes may include, but are not limited to, a protein, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, a gap junction, an adherens junction, or any combination thereof. In some instances, cell features may include intracellular analytes, such as proteins, protein modifications (e.g., phosphorylation status or other post-translational modifications), nuclear proteins, nuclear membrane proteins, or any combination thereof.

In some embodiments, an analyte binding moiety may include any molecule or moiety capable of binding to an analyte (e.g., a biological analyte, e.g., a macromolecular constituent). A labelling agent may include, but is not limited to, a protein, a peptide, an antibody (or an epitope binding fragment thereof), a lipophilic moiety (such as cholesterol), a cell surface receptor binding molecule, a receptor ligand, a small molecule, a bi-specific antibody, a bi-specific T-cell engager, a T-cell receptor engager, a B-cell receptor engager, a pro-body, an aptamer, a monobody, an affimer, a darpin, and a protein scaffold, or any combination thereof. The labelling agents can include (e.g., are attached to) a reporter oligonucleotide that is indicative of the cell surface feature to which the binding group binds. For example, the reporter oligonucleotide may comprise a barcode sequence that permits identification of the labelling agent. For example, a labelling agent that is specific to one type of cell feature (e.g., a first cell surface feature) may have coupled thereto a first reporter oligonucleotide, while a labelling agent that is specific to a different cell feature (e.g., a second cell surface feature) may have a different reporter oligonucleotide coupled thereto. For a description of exemplary labelling agents, reporter oligonucleotides, and methods of use, see, e.g., U.S. Pat. No. 10,550,429; U.S. Pat. Pub. 20190177800; and U.S. Pat. Pub. 20190367969, which are each incorporated by reference herein in their entirety.

In some embodiments, an analyte binding moiety includes one or more antibodies or antigen binding fragments thereof. The antibodies or antigen binding fragments including the analyte binding moiety can specifically bind to a target analyte. In some embodiments, the analyte is a protein (e.g., a protein on a surface of the biological sample (e.g., a cell) or an intracellular protein). In some embodiments, a plurality of analyte labelling agents comprising a plurality of analyte binding moieties bind a plurality of analytes present in a biological sample. In some embodiments, the plurality of analytes includes a single species of analyte (e.g., a single species of polypeptide). In some embodiments in which the plurality of analytes includes a single species of analyte, the analyte binding moieties of the plurality of analyte labelling agents are the same. In some embodiments in which the plurality of analytes includes a single species of analyte, the analyte binding moieties of the plurality of analyte labelling agents are the different (e.g., members of the plurality of analyte labelling agents can have two or more species of analyte binding moieties, wherein each of the two or more species of analyte binding moieties binds a single species of analyte, e.g., at different binding sites). In some embodiments, the plurality of analytes includes multiple different species of analyte (e.g., multiple different species of polypeptides).

In other instances, e.g., to facilitate sample multiplexing, a labelling agent that is specific to a particular cell feature may have a first plurality of the labelling agent (e.g., an antibody or lipophilic moiety) coupled to a first reporter oligonucleotide and a second plurality of the labelling agent coupled to a second reporter oligonucleotide.

In some aspects, these reporter oligonucleotides may comprise nucleic acid barcode sequences that permit identification of the labelling agent which the reporter oligonucleotide is coupled to. The selection of oligonucleotides as the reporter may provide advantages of being able to generate significant diversity in terms of sequence, while also being readily attachable to most biomolecules, e.g., antibodies, etc., as well as being readily detected, e.g., using sequencing or array technologies.

Attachment (coupling) of the reporter oligonucleotides to the labelling agents may be achieved through any of a variety of direct or indirect, covalent or non-covalent associations or attachments. For example, oligonucleotides may be covalently attached to a portion of a labelling agent (such a protein, e.g., an antibody or antibody fragment) using chemical conjugation techniques (e.g., Lightning-Link® antibody labelling kits available from Innova Biosciences), as well as other non-covalent attachment mechanisms, e.g., using biotinylated antibodies and oligonucleotides (or beads that include one or more biotinylated linker, coupled to oligonucleotides) with an avidin or streptavidin linker. Antibody and oligonucleotide biotinylation techniques are available. See, e.g., Fang, et al., “Fluoride-Cleavable Biotinylation Phosphoramidite for 5′-end-Labelling and Affinity Purification of Synthetic Oligonucleotides,” Nucleic Acids Res. Jan. 15, 2003; 31(2):708-715, which is entirely incorporated herein by reference for all purposes. Likewise, protein and peptide biotinylation techniques have been developed and are readily available. See, e.g., U.S. Pat. No. 6,265,552, which is entirely incorporated herein by reference for all purposes. Furthermore, click reaction chemistry may be used to couple reporter oligonucleotides to labelling agents. Commercially available kits, such as those from Thunderlink and Abcam, and techniques common in the art may be used to couple reporter oligonucleotides to labelling agents as appropriate. In another example, a labelling agent is indirectly (e.g., via hybridization) coupled to a reporter oligonucleotide comprising a barcode sequence that identifies the label agent. For instance, the labelling agent may be directly coupled (e.g., covalently bound) to a hybridization oligonucleotide that comprises a sequence that hybridizes with a sequence of the reporter oligonucleotide. Hybridization of the hybridization oligonucleotide to the reporter oligonucleotide couples the labelling agent to the reporter oligonucleotide. In some embodiments, the reporter oligonucleotides are releasable from the labelling agent, such as upon application of a stimulus. For example, the reporter oligonucleotide may be attached to the labeling agent through a labile bond (e.g., chemically labile, photolabile, thermally labile, etc.) as generally described for releasing molecules from supports elsewhere herein. In some instances, the reporter oligonucleotides described herein may include one or more functional sequences that can be used in subsequent processing, such as an adapter sequence, a unique molecular identifier (UMI) sequence, a sequencer specific flow cell attachment sequence (such as an P5, P7, or partial P5 or P7 sequence), a primer or primer binding sequence, a sequencing primer or primer binding sequence (such as an R1, R2, or partial R1 or R2 sequence).

In some cases, the labelling agent can comprise a reporter oligonucleotide and a label. A label can be fluorophore, a radioisotope, a molecule capable of a colorimetric reaction, a magnetic particle, or any other suitable molecule or compound capable of detection. The label can be conjugated to a labelling agent (or reporter oligonucleotide) either directly or indirectly (e.g., the label can be conjugated to a molecule that can bind to the labelling agent or reporter oligonucleotide). In some cases, a label is conjugated to a first oligonucleotide that is complementary (e.g., hybridizes) to a sequence of the reporter oligonucleotide.

In some embodiments, multiple different species of analytes (e.g., polypeptides) from the biological sample can be subsequently associated with the one or more physical properties of the biological sample. For example, the multiple different species of analytes can be associated with locations of the analytes in the biological sample. Such information (e.g., proteomic information when the analyte binding moiety(ies) recognizes a polypeptide(s)) can be used in association with other spatial information (e.g., genetic information from the biological sample, such as DNA sequence information, transcriptome information (e.g., sequences of transcripts), or both). For example, a cell surface protein of a cell can be associated with one or more physical properties of the cell (e.g., a shape, size, activity, or a type of the cell). The one or more physical properties can be characterized by imaging the cell. The cell can be bound by an analyte labelling agent comprising an analyte binding moiety that binds to the cell surface protein and an analyte binding moiety barcode that identifies that analyte binding moiety. Results of protein analysis in a sample (e.g., a tissue sample or a cell) can be associated with DNA and/or RNA analysis in the sample.

(iii) Products of Endogenous Analyte and/or Labelling Agent

In some embodiments, provided herein are methods and compositions for analyzing one or more products of an endogenous analyte and/or a labelling agent in a biological sample. In some embodiments, an endogenous analyte (e.g., a viral or cellular DNA or RNA) or a product (e.g., a hybridization product, a ligation product, an extension product (e.g., by a DNA or RNA polymerase), a replication product, a transcription/reverse transcription product, and/or an amplification product such as a rolling circle amplification (RCA) product) thereof is analyzed e.g., using a method that reduces the signals detected in a given detection step, round, or cycle, of decoding, as described in Section IV. In some embodiments, a labelling agent that directly or indirectly binds to an analyte in the biological sample is detected and analyzed. In some embodiments, a product (e.g., a hybridization product, a ligation product, an extension product (e.g., by a DNA or RNA polymerase), a replication product, a transcription/reverse transcription product, and/or an amplification product such as a rolling circle amplification (RCA) product) of a labelling agent that directly or indirectly binds to an analyte in the biological sample is analyzed.

(a) Hybridization

In some embodiments, a product of an endogenous analyte and/or a labelling agent is a hybridization product comprising the pairing of substantially complementary or complementary nucleic acid sequences within two different molecules, one of which is the endogenous analyte or the labelling agent (e.g., reporter oligonucleotide attached thereto). The other molecule can be another endogenous molecule or another labelling agent such as a probe. Pairing can be achieved by any process in which a nucleic acid sequence joins with a substantially or fully complementary sequence through base pairing to form a hybridization complex. For purposes of hybridization, two nucleic acid sequences are “substantially complementary” if at least 60% (e.g., at least 70%, at least 80%, or at least 90%) of their individual bases are complementary to one another.

Various probes and probe sets can be hybridized to an endogenous analyte and/or a labelling agent and each probe may comprise one or more barcode sequences. Exemplary barcoded probes or probe sets may be based on a padlock probe, a gapped padlock probe, a SNAIL (Splint Nucleotide Assisted Intramolecular Ligation) probe set, a PLAYR (Proximity Ligation Assay for RNA) probe set, a PLISH (Proximity Ligation in situ Hybridization) probe set, and RNA-templated ligation probes. The specific probe or probe set design can vary. Various probes and probe sets described herein or products thereof can be detected using a method that reduces the signals detected in a given detection step, round, or cycle, of decoding, as described in Section IV.

(b) Ligation

In some embodiments, a product of an endogenous analyte and/or a labelling agent is a ligation product. In some embodiments, the ligation product is formed between two or more endogenous analytes. In some embodiments, the ligation product is formed between an endogenous analyte and a labelling agent. In some embodiments, the ligation product is formed between two or more labelling agent. In some embodiments, the ligation product is an intramolecular ligation of an endogenous analyte. In some embodiments, the ligation product is an intramolecular ligation of a labelling agent, for example, the circularization of a circularizable probe or probe set upon hybridization to a target sequence. The target sequence can be comprised in an endogenous analyte (e.g., nucleic acid such as a genomic DNA or mRNA) or a product thereof (e.g., cDNA from a cellular mRNA transcript), or in a labelling agent (e.g., the reporter oligonucleotide) or a product thereof.

In some embodiments, provided herein is a probe or probe set capable of DNA-templated ligation, such as from a cDNA molecule. See, e.g., U.S. Pat. No. 8,551,710, which is hereby incorporated by reference in its entirety. In some embodiments, provided herein is a probe or probe set capable of RNA-templated ligation. See, e.g., U.S. Pat. Pub. 2020/0224244 which is hereby incorporated by reference in its entirety. In some embodiments, the probe set is a SNAIL probe set. See, e.g., U.S. Pat. Pub. 20190055594, which is hereby incorporated by reference in its entirety. In some embodiments, provided herein is a multiplexed proximity ligation assay. See, e.g., U.S. Pat. Pub. 20140194311 which is hereby incorporated by reference in its entirety. In some embodiments, provided herein is a probe or probe set capable of proximity ligation, for instance a proximity ligation assay for RNA (e.g., PLAYR) probe set. See, e.g., U.S. Pat. Pub. 20160108458, which is hereby incorporated by reference in its entirety. In some embodiments, a circular probe can be indirectly hybridized to the target nucleic acid. In some embodiments, the circular construct is formed from a probe set capable of proximity ligation, for instance a proximity ligation in situ hybridization (PLISH) probe set. See, e.g., U.S. Pat. Pub. 2020/0224243 which is hereby incorporated by reference in its entirety.

In some embodiments, the ligation involves chemical ligation (e.g., click chemistry ligation). In some embodiments, the ligation involves template dependent ligation. In some embodiments, the ligation involves template independent ligation. In some embodiments, the click reaction is a template-independent reaction (see, e.g., Xiong and Seela (2011), J. Org. Chem. 76(14): 5584-5597, incorporated by reference herein in its entirety). In some embodiments, the click reaction is a template-dependent reaction or template-directed reaction. In some embodiments, the template-dependent reaction is sensitive to base pair mismatches such that reaction rate is significantly higher for matched versus unmatched templates. In some embodiments, the click reaction is a nucleophilic addition template-dependent reaction. In some embodiments, the click reaction is a cyclopropane-tetrazine template-dependent reaction.

In some embodiments, the ligation involves enzymatic ligation. In some embodiments, the enzymatic ligation involves use of a ligase. In some aspects, the ligase used herein comprises an enzyme that is commonly used to join polynucleotides together or to join the ends of a single polynucleotide. An RNA ligase, a DNA ligase, or another variety of ligase can be used to ligate two nucleotide sequences together. Ligases comprise ATP-dependent double-strand polynucleotide ligases, NAD-i-dependent double-strand DNA or RNA ligases and single-strand polynucleotide ligases, for example any of the ligases described in EC 6.5.1.1 (ATP-dependent ligases), EC 6.5.1.2 (NAD+-dependent ligases), EC 6.5.1.3 (RNA ligases). Specific examples of ligases comprise bacterial ligases such as E. coli DNA ligase, Tth DNA ligase, Thermococcus sp. (strain 9° N) DNA ligase (9° N™ DNA ligase, New England Biolabs), Taq DNA ligase, Ampligase™ (Epicentre Biotechnologies) and phage ligases such as T3 DNA ligase, T4 DNA ligase and T7 DNA ligase and mutants thereof. In some embodiments, the ligase is a T4 RNA ligase. In some embodiments, the ligase is a splintR ligase. In some embodiments, the ligase is a single stranded DNA ligase. In some embodiments, the ligase is a T4 DNA ligase. In some embodiments, the ligase is a ligase that has a DNA-splinted DNA ligase activity. In some embodiments, the ligase is a ligase that has an RNA-splinted DNA ligase activity.

In some embodiments, the ligation herein is a direct ligation. In some embodiments, the ligation herein is an indirect ligation. “Direct ligation” means that the ends of the polynucleotides hybridize immediately adjacently to one another to form a substrate for a ligase enzyme resulting in their ligation to each other (intramolecular ligation). Alternatively, “indirect” means that the ends of the polynucleotides hybridize non-adjacently to one another, e.g., separated by one or more intervening nucleotides or “gaps”. In some embodiments, said ends are not ligated directly to each other, but instead occurs either via the intermediacy of one or more intervening (so-called “gap” or “gap-filling” (oligo)nucleotides) or by the extension of the 3′ end of a probe to “fill” the “gap” corresponding to said intervening nucleotides (intermolecular ligation). In some cases, the gap of one or more nucleotides between the hybridized ends of the polynucleotides may be “filled” by one or more “gap” (oligo)nucleotide(s) which are complementary to a splint, padlock probe, or target nucleic acid. The gap may be a gap of 1 to 60 nucleotides or a gap of 1 to 40 nucleotides or a gap of 3 to 40 nucleotides. In specific embodiments, the gap may be a gap of about 1, 2, 3, 4, 5, 6, 7, 8, 9 or 10 or more nucleotides, of any integer (or range of integers) of nucleotides in between the indicated values. In some embodiments, the gap between said terminal regions may be filled by a gap oligonucleotide or by extending the 3′ end of a polynucleotide. In some cases, ligation involves ligating the ends of the probe to at least one gap (oligo)nucleotide, such that the gap (oligo)nucleotide becomes incorporated into the resulting polynucleotide. In some embodiments, the ligation herein is preceded by gap filling. In other embodiments, the ligation herein does not require gap filling.

In some embodiments, ligation of the polynucleotides produces polynucleotides with melting temperature higher than that of unligated polynucleotides. Thus, in some aspects, ligation stabilizes the hybridization complex containing the ligated polynucleotides prior to subsequent steps, comprising amplification and detection.

In some aspects, a high fidelity ligase, such as a thermostable DNA ligase (e.g., a Taq DNA ligase), is used. Thermostable DNA ligases are active at elevated temperatures, allowing further discrimination by incubating the ligation at a temperature near the melting temperature (Tm) of the DNA strands. This selectively reduces the concentration of annealed mismatched substrates (expected to have a slightly lower Tm around the mismatch) over annealed fully base-paired substrates. Thus, high-fidelity ligation can be achieved through a combination of the intrinsic selectivity of the ligase active site and balanced conditions to reduce the incidence of annealed mismatched dsDNA.

In some embodiments, the ligation herein is a proximity ligation of ligating two (or more) nucleic acid sequences that are in proximity with each other, e.g., through enzymatic means (e.g., a ligase). In some embodiments, proximity ligation can include a “gap-filling” step that involves incorporation of one or more nucleic acids by a polymerase, based on the nucleic acid sequence of a template nucleic acid molecule, spanning a distance between the two nucleic acid molecules of interest (see, e.g., U.S. Pat. No. 7,264,929, the entire contents of which are incorporated herein by reference). A wide variety of different methods can be used for proximity ligating nucleic acid molecules, including (but not limited to) “sticky-end” and “blunt-end” ligations. Additionally, single-stranded ligation can be used to perform proximity ligation on a single-stranded nucleic acid molecule. Sticky-end proximity ligations involve the hybridization of complementary single-stranded sequences between the two nucleic acid molecules to be joined, prior to the ligation event itself. Blunt-end proximity ligations generally do not include hybridization of complementary regions from each nucleic acid molecule because both nucleic acid molecules lack a single-stranded overhang at the site of ligation.

(c) Primer Extension and Amplification

In some embodiments, a product is a primer extension product of an analyte, a labelling agent, a probe or probe set bound to the analyte (e.g., a circularizable probe such as a padlock probe bound to genomic DNA, mRNA, or cDNA), or a probe or probe set bound to the labelling agent (e.g., a circularizable probe such as a padlock probe bound to one or more reporter oligonucleotides from the same or different labelling agents).

A primer is generally a single-stranded nucleic acid sequence having a 3′ end that can be used as a substrate for a nucleic acid polymerase in a nucleic acid extension reaction. RNA primers are formed of RNA nucleotides, and are used in RNA synthesis, while DNA primers are formed of DNA nucleotides and used in DNA synthesis. Primers can also include both RNA nucleotides and DNA nucleotides (e.g., in a random or designed pattern). Primers can also include other natural or synthetic nucleotides described herein that can have additional functionality. In some examples, DNA primers can be used to prime RNA synthesis and vice versa (e.g., RNA primers can be used to prime DNA synthesis). Primers can vary in length. For example, primers can be about 6 bases to about 120 bases. For example, primers can include up to about 25 bases. A primer may, in some cases, refer to a primer binding sequence. A primer extension reaction generally refers to any method where two nucleic acid sequences become linked (e.g., hybridized) by an overlap of their respective terminal complementary nucleic acid sequences (e.g., 3′ termini). Such linking can be followed by nucleic acid extension (e.g., an enzymatic extension) of one, or both termini using the other nucleic acid sequence as a template for extension. Enzymatic extension can be performed by an enzyme including, but not limited to, a polymerase and/or a reverse transcriptase.

In some embodiments, a product of an endogenous analyte and/or a labelling agent is an amplification product of one or more polynucleotides, for instance, a circular probe or circularizable probe or probe set. In some embodiments, the amplifying is achieved by performing rolling circle amplification (RCA). In other embodiments, a primer that hybridizes to the circular probe or circularized probe is added and used as such for amplification. In some embodiments, the RCA comprises a linear RCA, a branched RCA, a dendritic RCA, or any combination thereof.

In some embodiments, the amplification is performed at a temperature between or between about 20° C. and about 60° C. In some embodiments, the amplification is performed at a temperature between or between about 30° C. and about 40° C. In some aspects, the amplification step, such as the rolling circle amplification (RCA) is performed at a temperature between at or about 25° C. and at or about 50° C., such as at or about 25° C., 27° C., 29° C., 31° C., 33° C., 35° C., 37° C., 39° C., 41° C., 43° C., 45° C., 47° C., or 49° C.

In some embodiments, upon addition of a DNA polymerase in the presence of appropriate dNTP precursors and other cofactors, a primer is elongated to produce multiple copies of the circular template. This amplification step can utilize isothermal amplification or non-isothermal amplification. In some embodiments, after the formation of the hybridization complex and association of the amplification probe, the hybridization complex is rolling-circle amplified to generate a cDNA nanoball (e.g., amplicon) containing multiple copies of the cDNA. Techniques for rolling circle amplification (RCA) are known in the art such as linear RCA, a branched RCA, a dendritic RCA, or any combination thereof (See, e.g., Baner et al, Nucleic Acids Research, 26:5073-5078, 1998; Lizardi et al, Nature Genetics 19:226, 1998; Mohsen et al., Acc Chem Res. 2016 Nov. 15; 49(11): 2540-2550; Schweitzer et al. Proc. Natl Acad. Sci. USA 97:101 13-1 19, 2000; Faruqi et al, BMC Genomics 2:4, 2000; Nallur et al, Nucl. Acids Res. 29:el 18, 2001; Dean et al. Genome Res. 1 1:l095-1099, 2001; Schweitzer et al, Nature Biotech. 20:359-365, 2002; U.S. Pat. Nos. 6,054,274, 6,291,187, 6,323,009, 6,344,329 and 6,368,801). Exemplary polymerases for use in RCA comprise DNA polymerase such phi29 (φ29) polymerase, Klenow fragment, Bacillus stearothermophilus DNA polymerase (BST), T4 DNA polymerase, T7 DNA polymerase, or DNA polymerase I. In some aspects, DNA polymerases that have been engineered or mutated to have desirable characteristics can be employed. In some embodiments, the polymerase is phi29 DNA polymerase.

In some aspects, during the amplification step, modified nucleotides can be added to the reaction to incorporate the modified nucleotides in the amplification product (e.g., nanoball). Exemplary of the modified nucleotides comprise amine-modified nucleotides. In some aspects of the methods, for example, for anchoring or cross-linking of the generated amplification product (e.g., nanoball) to a scaffold, to cellular structures and/or to other amplification products (e.g., other nanoballs). In some aspects, the amplification products comprises a modified nucleotide, such as an amine-modified nucleotide. In some embodiments, the amine-modified nucleotide comprises an acrylic acid N-hydroxysuccinimide moiety modification. Examples of other amine-modified nucleotides comprise, but are not limited to, a 5-Aminoallyl-dUTP moiety modification, a 5-Propargylamino-dCTP moiety modification, a N6-6-Aminohexyl-dATP moiety modification, or a 7-Deaza-7-Propargylamino-dATP moiety modification.

In some aspects, the polynucleotides and/or amplification product (e.g., amplicon) can be anchored to a polymer matrix. For example, the polymer matrix can be a hydrogel. In some embodiments, one or more of the polynucleotide probe(s) can be modified to contain functional groups that can be used as an anchoring site to attach the polynucleotide probes and/or amplification product to a polymer matrix. Exemplary modification and polymer matrix that can be employed in accordance with the provided embodiments comprise those described in, for example, WO 2014/163886, WO 2017/079406, US 2016/0024555, US 2018/0251833 and US 2017/0219465, which are herein incorporated by reference in their entireties. In some examples, the scaffold also contains modifications or functional groups that can react with or incorporate the modifications or functional groups of the probe set or amplification product. In some examples, the scaffold can comprise oligonucleotides, polymers or chemical groups, to provide a matrix and/or support structures.

The amplification products may be immobilized within the matrix generally at the location of the nucleic acid being amplified, thereby creating a localized colony of amplicons. The amplification products may be immobilized within the matrix by steric factors. The amplification products may also be immobilized within the matrix by covalent or noncovalent bonding. In this manner, the amplification products may be considered to be attached to the matrix. By being immobilized to the matrix, such as by covalent bonding or cross-linking, the size and spatial relationship of the original amplicons is maintained. By being immobilized to the matrix, such as by covalent bonding or cross-linking, the amplification products are resistant to movement or unraveling under mechanical stress.

In some aspects, the amplification products are copolymerized and/or covalently attached to the surrounding matrix thereby preserving their spatial relationship and any information inherent thereto. For example, if the amplification products are those generated from DNA or RNA within a cell embedded in the matrix, the amplification products can also be functionalized to form covalent attachment to the matrix preserving their spatial information within the cell thereby providing a subcellular localization distribution pattern. In some embodiments, the provided methods involve embedding the one or more polynucleotide probe sets and/or the amplification products in the presence of hydrogel subunits to form one or more hydrogel-embedded amplification products. In some embodiments, the hydrogel-tissue chemistry described comprises covalently attaching nucleic acids to in situ synthesized hydrogel for tissue clearing, enzyme diffusion, and multiple-cycle sequencing while an existing hydrogel-tissue chemistry method cannot. In some embodiments, to enable amplification product embedding in the tissue-hydrogel setting, amine-modified nucleotides are comprised in the amplification step (e.g., RCA), functionalized with an acrylamide moiety using acrylic acid N-hydroxysuccinimide esters, and copolymerized with acrylamide monomers to form a hydrogel.

In some embodiments, the RCA template may comprise the target analyte, or a part thereof, where the target analyte is a nucleic acid, or it may be provided or generated as a proxy, or a marker, for the analyte. In some embodiments, different analytes are detected in situ in one or more cells using a RCA-based detection system, e.g., where the signal is provided by generating an RCA product from a circular RCA template which is provided or generated in the assay, and the RCA product is detected to detect the corresponding analyte. The RCA product may thus be regarded as a reporter which is detected to detect the target analyte. However, the RCA template may also be regarded as a reporter for the target analyte; the RCA product is generated based on the RCA template, and comprises complementary copies of the RCA template. The RCA template determines the signal which is detected, and is thus indicative of the target analyte. As will be described in more detail below, the RCA template may be a probe, or a part or component of a probe, or may be generated from a probe, or it may be a component of a detection assay (e.g., a reagent in a detection assay), which is used as a reporter for the assay, or a part of a reporter, or signal-generation system. The RCA template used to generate the RCP may thus be a circular (e.g. circularized) reporter nucleic acid molecule, namely from any RCA-based detection assay which uses or generates a circular nucleic acid molecule as a reporter for the assay. Since the RCA template generates the RCP reporter, it may be viewed as part of the reporter system for the assay.

In some embodiments, a product herein includes a molecule or a complex generated in a series of reactions, e.g., hybridization, ligation, extension, replication, transcription/reverse transcription, and/or amplification (e.g., rolling circle amplification), in any suitable combination. For example, a product comprising a target sequence for a probe disclosed herein may be a hybridization complex formed of a cellular nucleic acid in a sample and an exogenously added nucleic acid probe. The exogenously added nucleic acid probe may comprise an overhang that does not hybridize to the cellular nucleic acid but hybridizes to another probe. The exogenously added nucleic acid probe may be optionally ligated to a cellular nucleic acid molecule or another exogenous nucleic acid molecule. In other examples, a product comprising a target sequence for a probe disclosed herein may be an RCP of a circularizable probe or probe set which hybridizes to a cellular nucleic acid molecule (e.g., genomic DNA or mRNA) or product thereof (e.g., a transcript such as cDNA, a DNA-templated ligation product of two probes, or an RNA-templated ligation product of two probes). In other examples, a product comprising a target sequence for a probe disclosed herein may a probe hybridizing to an RCP. The probe may comprise an overhang that does not hybridize to the RCP but hybridizes to another probe. The probe may be optionally ligated to a cellular nucleic acid molecule or another probe, e.g., an anchor probe that hybridize to the RCP.

C. Target Sequences

A target sequence for a probe disclosed herein may be comprised in any analyte disclose herein, including an endogenous analyte (e.g., a viral or cellular nucleic acid), a labelling agent, or a product of an endogenous analyte and/or a labelling agent.

In some aspects, one or more of the target sequences includes one or more barcode(s), e.g., at least two, three, four, five, six, seven, eight, nine, ten, or more barcodes. Barcodes can spatially-resolve molecular components found in biological samples, for example, within a cell or a tissue sample. A barcode can be attached to an analyte or to another moiety or structure in a reversible or irreversible manner. A barcode can be added to, for example, a fragment of a deoxyribonucleic acid (DNA) or ribonucleic acid (RNA) sample before or during sequencing of the sample. Barcodes can allow for identification and/or quantification of individual sequencing-reads (e.g., a barcode can be or can include a unique molecular identifier or “UMI”). In some aspects, a barcode comprises about 4, 5, 6, 7, 8, 9, 10, 11, 12, 13, 14, 15, 16, 17, 18, 19, 20, 21, 22, 23, 24, 25, 26, 27, 28, 29, 30, or more than 30 nucleotides.

In some embodiments, a barcode includes two or more sub-barcodes that together function as a single barcode. For example, a polynucleotide barcode can include two or more polynucleotide sequences (e.g., sub-barcodes) that are separated by one or more non-barcode sequences. In some embodiments, the one or more barcode(s) can also provide a platform for targeting functionalities, such as oligonucleotides, oligonucleotide-antibody conjugates, oligonucleotide-streptavidin conjugates, modified oligonucleotides, affinity purification, detectable moieties, enzymes, enzymes for detection assays or other functionalities, and/or for detection and identification of the polynucleotide.

In any of the preceding embodiments, barcodes (e.g., primary and/or secondary barcode sequences) can be analyzed (e.g., detected or sequenced) using any suitable methods or techniques, including those described herein, such as RNA sequential probing of targets (RNA SPOTs), sequential fluorescent in situ hybridization (seqFISH), single-molecule fluorescent in situ hybridization (smFISH), multiplexed error-robust fluorescence in situ hybridization (MERFISH), in situ sequencing, hybridization-based in situ sequencing (HybISS), targeted in situ sequencing, fluorescent in situ sequencing (FISSEQ), sequencing by synthesis (SBS), sequencing by ligation (SBL), sequencing by hybridization (SBH), or spatially-resolved transcript amplicon readout mapping (STARmap). In any of the preceding embodiments, the methods provided herein can include analyzing the barcodes by sequential hybridization and detection with a plurality of labelled probes (e.g., detection oligos).

In some embodiments, in a barcode sequencing method, barcode sequences are detected for identification of other molecules including nucleic acid molecules (DNA or RNA) longer than the barcode sequences themselves, as opposed to direct sequencing of the longer nucleic acid molecules. In some embodiments, a N-mer barcode sequence comprises 4^{N} complexity given a sequencing read of N bases, and a much shorter sequencing read may be required for molecular identification compared to non-barcode sequencing methods such as direct sequencing. For example, 1024 molecular species may be identified using a 5-nucleotide barcode sequence (4⁵=1024), whereas 8 nucleotide barcodes can be used to identify up to 65,536 molecular species, a number greater than the total number of distinct genes in the human genome. In some embodiments, the barcode sequences contained in the probes or RCPs are detected, rather than endogenous sequences, which can be an efficient read-out in terms of information per cycle of sequencing. Because the barcode sequences are pre-determined, they can also be designed to feature error detection and correction mechanisms, see, e.g., U.S. Pat. Pubs. 20190055594 and 20210164039, which are hereby incorporated by reference in their entirety.

### III. Signal Detection and Crowding

In some embodiments, the present disclosure addresses signal crowding in methods that involve detecting signals, e.g., from nucleic acid sequences (either as the target analytes or as the labels or reporters for one or more target analytes, such as one or more target proteins), including in situ assays that detect the localization of analytes in sample. There are a number of situations in which it is desired to detect several different analytes in a sample simultaneously, for example when detecting the expression of different genes in situ in a sample, where there can be a wide range of different expression levels. In some embodiments, nucleic acid molecules are detected as target analytes in situ in a sample. In some embodiments, nucleic acid molecules are detected as reporters for other, non-nucleic acid analytes, including for example proteins, or indeed as a reporter, or signal amplifier, for a nucleic acid analyte. Thus, in a detection assay for such an analyte, nucleic acid molecules may be used, for example as a tag or reporter for an antibody or other affinity-binder-based probe (e.g. in immunoPCR or immunoRCA), or generated, for example by ligation or extension in a proximity probe-based assay. For example, a proximity ligation reaction can include reporter oligonucleotides attached to pairs of antibodies that can be joined by ligation if the antibodies have been brought in proximity to each other, e.g., by binding the same target protein (complex), and the DNA ligation products that form are then used to template PCR amplification, as described for example in Soderberg et al., Methods (2008), 45(3): 227-32, the entire contents of which are incorporated herein by reference. In some embodiments, a proximity ligation reaction can include reporter oligonucleotides attached to antibodies that each bind to one member of a binding pair or complex, for example, for analyzing a binding between members of the binding pair or complex. For detection of analytes using oligonucleotides in proximity, see, e.g., U.S. Patent Application Publication No. 2002/0051986, the entire contents of which are incorporated herein by reference. In some embodiments, two analytes in proximity can be specifically bound by two labelling agents (e.g., antibodies) each of which is attached to a reporter oligonucleotide (e.g., DNA) that can participate, when in proximity when bound to their respective targets, in ligation, replication, and/or sequence decoding reactions. The nucleic acid molecule may be present in an amount which reflects the level of the analyte and may be detected as a “proxy” for the target analyte. Suitable methods for detecting multiple nucleic acid sequences in a sample are well known in the art, including the use of hybridization probes and sequencing-by-hybridization. Sequences of any suitable hybridization probes or products thereof can be analyzed (e.g., detected or sequenced) including those described herein, such as RNA sequential probing of targets (RNA SPOTs), sequential fluorescent in situ hybridization (seqFISH), single-molecule fluorescent in situ hybridization (smFISH), multiplexed error-robust fluorescence in situ hybridization (MERFISH), in situ sequencing, hybridization-based in situ sequencing (HybISS), or spatially-resolved transcript amplicon readout mapping (STARmap). For exemplary probes and probe sets, see, e.g., U.S. Pat. Pub. 2020/0224244, 2019/0055594, 2014/0194311, 2016/0108458, and 2020/0224243, each of which is hereby incorporated by reference in its entirety.

In some embodiments, a method disclosed herein comprises labelling analytes which are to be detected (either directly or indirectly) with detectable labels, using hybridization probes for example, and then detecting signals from those labels in order to identify the nucleic acid sequences. In some embodiments, some of the target nucleic acid sequences are present in the sample at significantly higher or lower concentrations than the other target nucleic acid sequences. If a particular target nucleic acid sequence is present in the sample at a high concentration, then a large number of hybridization probes will be bound to that target nucleic acid sequence and a large number of signals will be generated. In some embodiments, multiple signals are generated and detected concurrently, and the number of signals that are generated from each target nucleic acid sequence is related to the amount of that target nucleic acid sequence which is present in the sample. Accordingly, signals from target nucleic acid sequences which are present in high concentrations or in close proximity to signals from other target nucleic acid sequences may overcrowd and mask signals from the target nucleic acid sequences. In some embodiments, a method disclosed herein prevents and/or ameliorates signal crowding in multiplex assays where it is desired to detect a number of different nucleotide sequences, regardless of the means by which the sequences are labelled, and the type of labelling that is used (e.g. optical signals, radioactive signals, etc.). The present disclosure is particularly useful where a number of different signals are being generated simultaneously in close proximity.

In some embodiments, a method disclosed herein comprises detecting and identifying RNA transcripts in a given cell, in order to analyze the gene expression of that cell. In some embodiments, a method disclosed herein comprises labelling the RNA transcripts (or one or more primary or higher order probes bound thereto) with fluorescently labelled probes. The signals from the fluorescent labels can then be visualized in order to determine which RNA transcripts are present in a given cell of, e.g., a tissue sample. This can also be used to provide information on the location and the relative quantities of different RNA transcripts (and therefore the location and relative levels of expression of the corresponding genes). If a particular gene (or genes) is significantly overexpressed, a large number of RNA transcripts corresponding to that gene will be present in the sample, and thus a large number of fluorescent signals indicating the presence of that RNA transcript will be generated. At a certain point, the signal density will be such that at least some individual signals cannot be resolved using conventional fluorescence microscopy, thereby inhibiting or even preventing the detection of signals from other RNA transcripts corresponding to genes which are expressed at a lower level or that physically overlap or are otherwise in close proximity in the sample (either in 2D or 3D space), which leads to a loss of information and an inaccurate picture of gene expression. It will be understood that this problem can occur in many other nucleic acid detection methods. In some aspects, the present disclosure provides a method of detecting multiple analytes (e.g., target nucleic acid sequences) in a sample wherein signal crowding is reduced.

In some embodiments, the methods provided in this disclosure are for use in the multiplexed detection of analytes (such as nucleic acids), that is, for the detection of multiple target analytes in a sample, e.g., one or more tissue samples such as a single tissue section or a series of tissue sections. In some embodiments, the methods use hybridization probes, whilst reducing signal crowding from said hybridization probes. In some embodiments, the methods provided herein comprise sequencing-by-hybridization (SBH) for detecting nucleic acid sequences in a sample, including multiplex SBH for detecting different target nucleic acid sequences (e.g., labels or reporters for one or more target analytes), with a wide range of distribution and abundance simultaneously in a sample. In some embodiments, the methods provided herein comprise multiple rounds of probe hybridization and detection for each nucleic acid sequence in a sample. In some embodiments, the methods provided herein address signal crowding issues due to signals indicative of target nucleic acid sequences present in high concentrations and/or close proximity that may mask and/or overcrowd other signals. In some cases, signal overcrowding may introduce a mismatch in the observed signal and expected signal, resulting in a signal that cannot be associated with its corresponding analyte.

In some aspects, signal overcrowding may prevent signals relating to the target nucleic acid sequences from being generated, detected, analyzed and/or otherwise distinguished from other signals in the sample. For example, if the hybridization probes cannot successfully hybridize to their cognate target nucleic acid sequences due to steric hindrances, or if detection probes cannot hybridize to the hybridization probes, then signals will not be generated and thus the target nucleic acid sequences will not be detected. This may be referred to as steric crowding. In some cases, it may be that signals are properly generated from all of the target nucleic acid sequences, but that so many signals are generated, either in a particular area of the sample or in the sample as a whole (e.g., the signal density is too large), that not all of the signals can be properly detected and resolved. Where the signals are detected by optical means, this may be referred to as optical crowding, and the present methods are particularly suited to resolving, or reducing, optical crowding. In some aspects, by “optical means” is meant that the signals are detected visually, or by visual means, namely that the signals are visualized. Thus, in some instances, the signals that are generated involve detection of light or other visually detectable electromagnetic radiation (such as fluorescence). In some aspects, the signals may be optical signals, visual signals, or visually detectable signals. The signals may be detected by sight, typically after magnification, but more typically they are detected and analyzed in an automated system for the detection of the signals.

In some aspects, the signals may be detected by microscopy. In some aspects, an image may be generated in which the signals may be seen and detected, for example an image of the field of view of a microscope, or an image obtained from a camera. The signals may be detected by imaging, more particularly by imaging the sample or a part or reaction mixture thereof. By way of example, signals in an image may be detected as “spots” which can be seen in the image. In some aspects, a signal may be seen as a spot in an image. In some aspects, optical crowding occurs when individual spots cannot be resolved, or distinguished from one another, for example when they overlap, or mask one another. By reducing the number of spots using the methods herein, such that individual spots, or signals, can be resolved, optical crowding can be reduced. In some aspects, the present methods optically de-crowd the signals.

In some embodiments, the analyzing the sequence comprises imaging the sample (e.g., an amplification product or associated probes). In some cases, analysis is performed on one or more images captured, and may comprise processing the image(s) and/or quantifying signals observed. In some embodiments, images of signals from different fluorescent channels and/or detectable probe hybridization cycles can be compared and analyzed. In some embodiments, images of signals (or absence thereof) at a particular location in a sample from different fluorescent channels and/or sequential detectable probe hybridization cycles can be aligned to analyze an analyte at the location. For instance, a particular location in a sample can be tracked and signal spots from sequential sequencing-by-ligation cycles can be analyzed to detect a target polynucleotide sequence (e.g., a barcode sequence or subsequence thereof) in a nucleic acid at the location. The analysis may comprise processing information of one or more cell types, one or more types of analytes, a number or level of analyte, and/or a number or level of cells detected in a particular region of the sample. In some embodiments, the analysis comprises detecting a sequence e.g., a barcode sequence present in an amplification product at a location in the sample. In some embodiments, the analysis includes quantification of puncta (e.g., if amplification products are detected). In some cases, the analysis includes determining whether particular cells and/or signals are present that correlate with one or more analytes from a particular panel. In some embodiments, the obtained information may be compared to a positive and negative control, or to a threshold of a feature to determine if the sample exhibits a certain feature or phenotype. In some cases, the information may comprise signals from a cell, a region, and/or comprise readouts from multiple detectable labels. In some case, the analysis further includes displaying the information from the analysis or detection step. In some embodiments, software may be used to automate the processing, analysis, and/or display of data.

In some aspects, the reduction in signal crowding associated with the present methods may be considered to be a reduction relative to the level of signal crowding which would occur in a method which did not comprise steps to reduce signal crowding, for example, without reducing the number of targets detected in a given cycle, such as by the omission of particular hybridization probes described herein.

In some aspects, the methods herein involve reducing the number of signals that are detected in a detection step of the method. This is achieved in different ways in the different methods, to prevent or block a signal from being generated from certain (e.g., pre-selected or randomly or combinatorially designated) targets, and/or in the context of a sequential detection scheme comprising a number of detection cycles, to omit (e.g., pre-selected or randomly or combinatorially designated) target(s) from detection in a given cycle of detection, such that a reduced number of targets is detected in a given cycle.

### IV. Signal Amplification and Sparse Labelling

### A. Signal Amplification In Situ

In some embodiments, the present disclosure relates to the detection of analytes (e.g., nucleic acids sequences) in situ using probe hybridization. In some embodiments, the detection of analytes comprises generation of amplified signals associated with the probes.

In some embodiments, detection comprises an amplification product of one or more polynucleotides, for instance, a circular probe or circularizable probe or probe set hybridized to an analyte. In some embodiments, the amplifying is achieved as described in Section II.B, e.g., by performing extension or amplification such as rolling circle amplification (RCA).

Exemplary signal amplification methods include targeted deposition of detectable reactive molecules around the site of probe hybridization, targeted assembly of branched structures (e.g., bDNA or branched assay using locked nucleic acid (LNA)), programmed in situ growth of concatemers by enzymatic rolling circle amplification (RCA) (e.g., as described in US 2019/0055594 incorporated herein by reference), hybridization chain reaction, assembly of topologically catenated DNA structures using serial rounds of chemical ligation (clampFISH), signal amplification via hairpin-mediated concatemerization (e.g., as described in US 2020/0362398 incorporated herein by reference), e.g., primer exchange reactions such as signal amplification by exchange reaction (SABER) or SABER with DNA-Exchange (Exchange-SABER), or any combination thereof. In some embodiments, non-enzymatic signal amplification may be used. In some embodiments, an amplification product of any of the amplification methods provided herein (e.g., comprising a barcode sequence that uniquely corresponds to a particular analyte) can be analyzed using a method that reduces the signals detected in a given detection step, round, or cycle, of decoding, as described in Section IV.B.

The detectable reactive molecules may comprise tyramide, such as used in tyramide signal amplification (TSA) or multiplexed catalyzed reporter deposition (CARD)-FISH. In some embodiments, the detectable reactive molecule may be releasable and/or cleavable from a detectable label such as a fluorophore. In some embodiments, a method disclosed herein comprises multiplexed analysis of a biological sample comprising consecutive cycles of probe hybridization, fluorescence imaging, and signal removal, where the signal removal comprises removing the fluorophore from a fluorophore-labeled reactive molecule (e.g., tyramide). Exemplary detectable reactive reagents and methods are described in U.S. Pat. No. 6,828,109, US 2019/0376956, WO 2019/236841, US 2022/0026433, US 2022/0128565, and US 2021/0222234, all of which are incorporated herein by reference in their entireties.

In some embodiments, hybridization chain reaction (HCR) can be used for signal amplification. HCR is an enzyme-free nucleic acid amplification based on a triggered chain of hybridization of nucleic acid molecules starting from HCR monomers, which hybridize to one another to form a nicked nucleic acid polymer. This polymer is the product of the HCR reaction which is ultimately detected in order to indicate the presence of the target analyte. HCR is described in detail in Dirks and Pierce, 2004, PNAS, 101(43), 15275-15278 and in U.S. Pat. No. 7,632,641 and U.S. Pat. No. 7,721,721 (see also US 2006/00234261; Chemeris et al, 2008 Doklady Biochemistry and Biophysics, 419, 53-55; Niu et al, 2010, 46, 3089-3091; Choi et al, 2010, Nat. Biotechnol. 28(11), 1208-1212; and Song et al, 2012, Analyst, 137, 1396-1401). HCR monomers typically comprise a hairpin, or other metastable nucleic acid structure. In the simplest form of HCR, two different types of stable hairpin monomer, referred to here as first and second HCR monomers, undergo a chain reaction of hybridization events to form a long nicked double-stranded DNA molecule when an “initiator” nucleic acid molecule is introduced. The HCR monomers have a hairpin structure comprising a double stranded stem region, a loop region connecting the two strands of the stem region, and a single stranded region at one end of the double stranded stem region. The single stranded region which is exposed (and which is thus available for hybridization to another molecule, e.g. initiator or other HCR monomer) when the monomers are in the hairpin structure may be known as the “toehold region” (or “input domain”). The first HCR monomers each further comprise a sequence which is complementary to a sequence in the exposed toehold region of the second HCR monomers. This sequence of complementarity in the first HCR monomers may be known as the “interacting region” (or “output domain”). Similarly, the second HCR monomers each comprise an interacting region (output domain), e.g. a sequence which is complementary to the exposed toehold region (input domain) of the first HCR monomers. In the absence of the HCR initiator, these interacting regions are protected by the secondary structure (e.g. they are not exposed), and thus the hairpin monomers are stable or kinetically trapped (also referred to as “metastable”), and remain as monomers (e.g. preventing the system from rapidly equilibrating), because the first and second sets of HCR monomers cannot hybridize to each other. However, once the initiator is introduced, it is able to hybridize to the exposed toehold region of a first HCR monomer, and invade it, causing it to open up. This exposes the interacting region of the first HCR monomer (e.g. the sequence of complementarity to the toehold region of the second HCR monomers), allowing it to hybridize to and invade a second HCR monomer at the toehold region. This hybridization and invasion in turn opens up the second HCR monomer, exposing its interacting region (which is complementary to the toehold region of the first HCR monomers), and allowing it to hybridize to and invade another first HCR monomer. The reaction continues in this manner until all of the HCR monomers are exhausted (e.g. all of the HCR monomers are incorporated into a polymeric chain). Ultimately, this chain reaction leads to the formation of a nicked chain of alternating units of the first and second monomer species. The presence of the HCR initiator is thus required in order to trigger the HCR reaction by hybridization to and invasion of a first HCR monomer. The first and second HCR monomers are designed to hybridize to one another are thus may be defined as cognate to one another. They are also cognate to a given HCR initiator sequence. HCR monomers which interact with one another (hybridize) may be described as a set of HCR monomers or an HCR monomer, or hairpin, system.

An HCR reaction could be carried out with more than two species or types of HCR monomers. For example, a system involving three HCR monomers could be used. In such a system, each first HCR monomer may comprise an interacting region which binds to the toehold region of a second HCR monomer; each second HCR may comprise an interacting region which binds to the toehold region of a third HCR monomer; and each third HCR monomer may comprise an interacting region which binds to the toehold region of a first HCR monomer. The HCR polymerization reaction would then proceed as described above, except that the resulting product would be a polymer having a repeating unit of first, second and third monomers consecutively. Corresponding systems with larger numbers of sets of HCR monomers could readily be conceived. Branching HCR systems have also been devised and described (see, e.g., US 2022/0064697 incorporated herein by reference), and may be used in the methods herein.

In some embodiments, similar to HCR reactions that use hairpin monomers, linear oligo hybridization chain reaction (LO-HCR) can also be used for signal amplification. In some embodiments, provided herein is a method of detecting an analyte in a sample comprising: (i) performing a linear oligo hybridization chain reaction (LO-HCR), wherein an initiator is contacted with a plurality of LO-HCR monomers of at least a first and a second species to generate a polymeric LO-HCR product hybridized to a target nucleic acid molecule, wherein the first species comprises a first hybridization region complementary to the initiator and a second hybridization region complementary to the second species, wherein the first species and the second species are linear, single-stranded nucleic acid molecules; wherein the initiator is provided in one or more parts, and hybridizes directly or indirectly to or is comprised in the target nucleic acid molecule; and (ii) detecting the polymeric product, thereby detecting the analyte. In some embodiments, the first species and/or the second species may not comprise a hairpin structure. In some embodiments, the plurality of LO-HCR monomers may not comprise a metastable secondary structure. In some embodiments, the LO-HCR polymer may not comprise a branched structure. In some embodiments, performing the linear oligo hybridization chain reaction comprises contacting the target nucleic acid molecule with the initiator to provide the initiator hybridized to the target nucleic acid molecule. In any of the embodiments herein, the target nucleic acid molecule and/or the analyte can be an RCA product.

In some embodiments, detection of nucleic acids sequences in situ includes an assembly for branched signal amplification. In some embodiments, the assembly complex comprises an amplifier hybridized directly or indirectly (via one or more oligonucleotides) to a sequence (e.g., any of the barcodes described herein) present in the primary and secondary probes described herein and/or in a product or derivative thereof. In some embodiments, the assembly includes one or more amplifiers each including an amplifier repeating sequence. In some aspects, the one or more amplifiers is labeled. Described herein is a method of using the aforementioned assembly, including for example, using the assembly in multiplexed error-robust fluorescent in situ hybridization (MERFISH) applications, with branched DNA amplification for signal readout. In some embodiments, the amplifier repeating sequence is about 5-30 nucleotides, and is repeated N times in the amplifier. In some embodiments, the amplifier repeating sequence is about 20 nucleotides, and is repeated at least two times in the amplifier. In some aspects, the one or more amplifier repeating sequence is labeled. For exemplary branched signal amplification, see e.g., U.S. Pat. Pub. No. US20200399689A1 and Xia et al., Multiplexed Detection of RNA using MERFISH and branched DNA amplification. Scientific Reports (2019), each of which is fully incorporated by reference herein.

In some embodiments, the sequences (e.g., any of the barcodes described herein) present in the primary and second probes described herein and/or in a product or derivative thereof can be detected in with a method that comprises signal amplification by performing a primer exchange reaction (PER). In various embodiments, a primer with domain on its 3′ end binds to a catalytic hairpin, and is extended with a new domain by a strand displacing polymerase. For example, a primer with domain 1 on its 3′ ends binds to a catalytic hairpin, and is extended with a new domain 1 by a strand displacing polymerase, with repeated cycles generating a concatemer of repeated domain 1 sequences. In various embodiments, the strand displacing polymerase is Bst. In various embodiments, the catalytic hairpin includes a stopper which releases the strand displacing polymerase. In various embodiments, branch migration displaces the extended primer, which can then dissociate. In various embodiments, the primer undergoes repeated cycles to form a concatemer primer. In various embodiments, the sample may be contacted with a plurality of concatemer primers and a plurality of labeled probes. see e.g., U.S. Pat. Pub. No. US20190106733, which is incorporated herein by reference, for exemplary molecules and PER reaction components.

In some embodiments, provided herein are probes, probe sets, and assay methods to couple target nucleic acid detection, signal amplification (e.g., through nucleic acid amplification such as RCA, and/or hybridization of a plurality of detectably labeled probes, such as in hybridization chain reactions and the like), and decoding of the barcodes.

In some aspects, a primary probe, a secondary probe, and/or a higher order probe can be selected from the group consisting of a circular probe, a circularizable probe, and a linear probe. In some embodiments, a circular probe can be one that is pre-circularized prior to hybridization to a target nucleic acid and/or one or more other probes. In some embodiments, a circularizable probe can be one that can be circularized upon hybridization to a target nucleic acid and/or one or more other probes such as a splint. In some embodiments, a linear probe can be one that comprises a target recognition sequence and a sequence that does not hybridize to a target nucleic acid, such as a 5′ overhang, a 3′ overhang, and/or a linker or spacer (which may comprise a nucleic acid sequence or a non-nucleic acid moiety). In some embodiments, the sequence (e.g., the 5′ overhang, 3′ overhang, and/or linker or spacer) is non-hybridizing to the target nucleic acid but may hybridize to one another and/or one or more other probes, such as detectably labeled probes.

Specific probe designs can vary depending on the application. For instance, a primary probe, a secondary probe, and/or a higher order probe disclosed herein can comprise a padlock probe that does require gap filling to circularize upon hybridization to a template (e.g., a target nucleic acid and/or a probe such as a splint), a gapped padlock probe (e.g., one that require gap filling to circularize upon hybridization to a template), an L-shaped probe (e.g., one that comprises a target recognition sequence and a 5′ or 3′ overhang upon hybridization to a target nucleic acid or a probe), a U-shaped probe (e.g., one that comprises a target recognition sequence, a 5′ overhang, and a 3′ overhang upon hybridization to a target nucleic acid or a probe), a V-shaped probe (e.g., one that comprises at least two target recognition sequences and a linker or spacer between the target recognition sequences upon hybridization to a target nucleic acid or a probe), a probe or probe set for proximity ligation (such as those described in U.S. Pat. No. 7,914,987 and U.S. Pat. No. 8,580,504 incorporated herein by reference in their entireties, and probes for Proximity Ligation Assay (PLA) for the simultaneous detection and quantification of nucleic acid molecules and protein-protein interactions), or any suitable combination thereof. A target recognition sequence can be any sequence that binds directly or indirectly to a nucleic acid sequence in or associated with a target analyte (e.g., a sequence capable of hybridizing to the nucleic acid sequence, such as a sequence complementary to the nucleic acid sequence). In some embodiments, a target recognition sequence is between about 4 and about 50 nucleotides in length (e.g., between about 10 and about 40, between about 10 and about 30, or between about 15 and about 25 nucleotides in length). In some embodiments, a primary probe, a secondary probe, and/or a higher order probe disclosed herein can comprise a padlock-like probe or probe set. In some embodiments, a nucleic acid probe disclosed herein is part of a SNAIL (Splint Nucleotide Assisted Intramolecular Ligation) probe set, such as one described in US 2019/0055594 or US 2021/0164039 which are incorporated herein by reference in their entireties. In some embodiments, a nucleic acid probe disclosed herein is part of a PLAYR (Proximity Ligation Assay for RNA) probe set, such as one described in US 2016/0108458 which is incorporated herein by reference in its entirety. In some embodiments, a nucleic acid probe disclosed herein is part of a PLISH (Proximity Ligation in situ hybridization) probe set, such as one described in US 2020/0224243 which is incorporated herein by reference in its entirety. Any suitable combination of the probe designs described herein can be used.

Any suitable circularizable probe or probe set, or indeed more generally circularizable reporter molecules, may be used to generate the RCA template which is used to generate the RCA product. By “circularizable” is meant that the probe or reporter (the RCA template) is in the form of a linear molecule having ligatable ends which may circularized by ligating the ends together directly or indirectly to each other, or to the respective ends of an intervening (“gap”) oligonucleotide or to an extended 3′ end of the circularizable RCA template. A circularizable template may also be provided in two or more parts, namely two or more molecules (e.g., oligonucleotides) which may be ligated together to form a circle. When said RCA template is circularizable it is circularized by ligation prior to RCA. Ligation may be templated using a ligation template, and in the case of padlock and molecular inversion probes and such like the target analyte may provide the ligation template, or it may be separately provided. The circularizable RCA template (or template part or portion) will comprise at its respective 3′ and 5′ ends regions of complementarity to corresponding cognate complementary regions (or binding sites) in the ligation template, which may be adjacent where the ends are directly ligated to each other, or non-adjacent, with an intervening “gap” sequence, where indirect ligation is to take place.

In the case of padlock probes, in one embodiment the ends of the padlock probe may be brought into proximity to each other by hybridization to adjacent sequences on a target nucleic acid molecule (such as a target analyte), which acts as a ligation template, thus allowing the ends to be ligated together to form a circular nucleic acid molecule, allowing the circularized padlock probe to act as a template for an RCA reaction. In such an example the terminal sequences of the padlock probe which hybridize to the target nucleic acid molecule will be specific to the target analyte in question, and will be replicated repeatedly in the RCA product. They may therefore act as a marker sequence indicative of that target analyte. Accordingly, it can be seen that the marker sequence in the RCA product may be equivalent to a sequence present in the target analyte itself. Alternatively, a marker sequence (e.g. tag or barcode sequence) may be provided in the non-target complementary parts of the padlock probe. In still a further embodiment, the marker sequence may be present in the gap oligonucleotide which is hybridized between the respective hybridized ends of the padlock probe, where they are hybridized to non-adjacent sequences in the target molecule. Such gap-filling padlock probes are akin to molecular inversion probes.

In some embodiments, similar circular RCA template molecules can be generated using molecular inversion probes. Like padlock probes, these are also typically linear nucleic acid molecules capable of hybridizing to a target nucleic acid molecule (such as a target analyte) and being circularized. The two ends of the molecular inversion probe may hybridize to the target nucleic acid molecule at sites which are proximate but not directly adjacent to each other, resulting in a gap between the two ends. The size of this gap may range from only a single nucleotide in some embodiments, to larger gaps of 100 to 500 nucleotides, or longer, in other embodiments. Accordingly, it is necessary to supply a polymerase and a source of nucleotides, or an additional gap-filling oligonucleotide, in order to fill the gap between the two ends of the molecular inversion probe, such that it can be circularized.

As with the padlock probe, the terminal sequences of the molecular inversion probe which hybridize to the target nucleic acid molecule, and the sequence between them, will be specific to the target analyte in question, and will be replicated repeatedly in the RCA product. They may therefore act as a marker sequence indicative of that target analyte. Alternatively, a marker sequence (e.g. tag or barcode sequence) may be provided in the non-target complementary parts of the molecular inversion probe.

In some embodiments, the probes disclosed herein may be invader probes, e.g., for generating a circular nucleic acid such as a circularized probe. Such probes are of particular utility in the detection of single nucleotide polymorphisms. The detection method of the present disclosure may, therefore, be used in the detection of a single nucleotide polymorphism, or indeed any variant base, in the target nucleic acid sequence. Probes for use in such a method may be designed such that the 3′ ligatable end of the probe is complementary to and capable of hybridizing to the nucleotide in the target molecule which is of interest (the variant nucleotide), and the nucleotide at the 3′ end of the 5′ additional sequence at the 5′ end of the probe or at the 5′ end of another, different, probe part is complementary to the same said nucleotide, but is prevented from hybridizing thereto by a 3′ ligatable end (e.g., it is a displaced nucleotide). Cleavage of the probe to remove the additional sequence provides a 5′ ligatable end, which may be ligated to the 3′ ligatable end of the probe or probe part if the 3′ ligatable end is hybridized correctly to (e.g., is complementary to) the target nucleic acid molecule. Probes designed according to this principle provide a high degree of discrimination between different variants at the position of interest, as only probes in which the 3′ ligatable end is complementary to the nucleotide at the position of interest may participate in a ligation reaction. In one embodiment, the probe is provided in a single part, and the 3′ and 5′ ligatable ends are provided by the same probe. In some embodiments, an invader probe is a padlock probe (an invader padlock or “iLock”), e.g., as described in Krzywkowski et al., Nucleic Acids Research 45, e161, 2017 and US 2020/0224244, which are incorporated herein by reference.

Other types of probe which result in circular molecules which can be detected by RCA and which comprise either a target analyte sequence or a complement thereof have been developed by Olink Bioscience (now Navinci Diagnostics AB) and include the Selector-type probes described in US 2019/0144940, which comprise sequences capable of directing the cleavage of a target nucleic acid molecule (e.g., a target analyte) so as to release a fragment comprising a target sequence from the target analyte and sequences capable of templating the circularization and ligation of the fragment. US 2019/0144940 describes probes which comprise a 3′ sequence capable of hybridizing to a target nucleic acid molecule (e.g., a target analyte) and acting as a primer for the production of a complement of a target sequence within the target nucleic acid molecule (e.g., by target templated extension of the primer), and an internal sequence capable of templating the circularization and ligation of the extended probe comprising the reverse complement of the target sequence within the target analyte and a portion of the probe. In the case of both such probes, target sequences or complements thereof are incorporated into a circularized molecule which acts as the template for the RCA reaction to generate the RCA product, which consequently comprises concatenated repeats of said target sequence. Again, said target sequence may act as, or may comprise a marker sequence within the RCA product indicative of the target analyte in question. Alternatively, a marker sequence (e.g. tag or barcode sequence) may be provided in the non-target complementary parts of the probes.

In some embodiments, when the RCA products of a plurality of different mRNA and/or cDNA analytes can be analyzed, a barcode sequence in a particular circular or padlock probe can uniquely correspond to a particular mRNA or cDNA analyte, and the particular circular or padlock probe can further comprise an anchor sequence that is common among circular or padlock probes for a subset of the plurality of different mRNA and/or cDNA analytes. In some embodiments, the first and/or second probes disclosed herein can further comprise an anchor sequence. In some embodiments, the anchor sequence or complement thereof in RCA products can be detected using detectable probes, e.g., an immediate probe (e.g., an L-shaped probe) that hybridizes to the anchor sequence or complement thereof and a fluorescently-labeled probe that hybridizes to immediate probe. Signals associated with the anchor sequence can be used to detect all RCA products that comprise the common anchor sequence or complement thereof. Thus, in some embodiments, signals associated with the anchor sequence can be used as controls during sequential cycles of detecting the target-specific barcode sequences (or complements thereof) in a plurality of RCA products.

In some embodiments, a nucleic acid probe disclosed herein can be pre-assembled from multiple components, e.g., prior to contacting the nucleic acid probe with a target nucleic acid or a sample. In some embodiments, a nucleic acid probe disclosed herein can be assembled during and/or after contacting a target nucleic acid or a sample with multiple components. In some embodiments, a nucleic acid probe disclosed herein is assembled in situ in a sample. In some embodiments, the multiple components can be contacted with a target nucleic acid or a sample in any suitable order and any suitable combination. For instance, a first component and a second component can be contacted with a target nucleic acid, to allow binding between the components and/or binding between the first and/or second components with the target nucleic acid. Optionally a reaction involving either or both components and/or the target nucleic acid, between the components, and/or between either one or both components and the target nucleic acid can be performed, such as hybridization, ligation, primer extension and/or amplification, chemical or enzymatic cleavage, click chemistry, or any combination thereof. In some embodiments, a third component can be added prior to, during, or after the reaction. In some embodiments, a third component can be added prior to, during, or after contacting the sample with the first and/or second components. In some embodiments, the first, second, and third components can be contacted with the sample in any suitable combination, sequentially or simultaneously. In some embodiments, the nucleic acid probe can be assembled in situ in a stepwise manner, each step with the addition of one or more components, or in a dynamic process where all components are assembled together. One or more removing steps, e.g., by washing the sample such as under stringent conditions, may be performed at any point during the assembling process to remove or destabilize undesired intermediates and/or components at that point and increase the chance of accurate probe assembly and specific target binding of the assembled probe.

### B. Sparse Labelling

Signal crowding can occur during the detection of these signals in highly multiplexed systems, or systems where there are a large number of signals. In accordance with the general principle outlined above of reducing the signals which are detected in a given detection step, the present inventors have developed a method with a combinatorial labelling and sequential decoding scheme in which signals from certain target nucleic acid sequences are not detected in given rounds, or cycles, of decoding. Thus, the decoding scheme includes so-called “dark” rounds or cycles, wherein a target analyte or probe thereto is not detected (“rendered” or “read”) in that round or cycle, e.g., it is omitted from the detection step, or binding (e.g., hybridization) and/or detection of a probe to the target analyte is blocked by an interfering agent. In this way, signal crowding is reduced by detecting signals from certain selected target nucleic acid sequences over an increased time period, e.g., over an increased number of decoding cycles. As the signals from the detectable labels of the hybridization probes are detected less frequently, this method may be referred to as “sparse labelling”. In some aspects, the detection of the signals from the hybridization probes is effectively spread or spaced out over time, which means that fewer signals are being detected at once, and therefore signal crowding is reduced. In some cases, wherein amplification is performed (e.g., as described in Section IV.A.), while signal amplification can increase signal-to-noise ratios (e.g., noise may be due to background signals such as autofluorescence in a sample) and increase detection sensitivity, signal amplification associated with highly expressed or abundant analytes in a sample may also exacerbate issues associated with crowding, such as those due to overlapping signals. In some cases, sparse labelling is useful for decoding of amplified signals. In some cases, sparse labelling is used in combination with one or more other approaches to decode amplified signals.

In some embodiments, a method disclosed herein comprises performing multiple cycles of decoding using a plurality of probes (e.g., using the probes in a pre-determined order), wherein each cycle comprises contacting the analytes (e.g., a signal amplification product such as rolling circle amplification products) with the probes, allowing the probes to bind to their respective analytes, and detecting a signal from the probes which have bound to the analytes, wherein in one or more cycles, the probes for one or more analytes (e.g., selected target nucleic acid sequences), and/or the detectable labels thereof, are omitted, such that signals from the one or more analytes are not detected in those cycles, thereby reducing signal crowding. In some embodiments, the bound (e.g., hybridized) probes and/or the detectable labels thereof are removed between cycles.

In some embodiments, a dark cycle for a given endogenous analyte may be achieved by omitting the probe for the given analyte in that cycle, or by contacting the sample with an interfering agent for that analyte or probe. In some instances, probes are omitted or blocked from binding the endogenous target analyte. In some instances, probes are omitted or blocked from binding a primary probe bound to the endogenous target analyte. In some instances, probes are omitted or blocked from binding an intermediate probe bound to a primary probe that is bound to the endogenous target analyte. In some instances, probes are omitted or blocked from binding an intermediate probe bound to an amplification product generated using a primary probe that is bound to the endogenous target analyte.

In some embodiments, a method disclosed herein comprises performing multiple cycles of decoding using a plurality of probes (e.g., using the probes in a pre-determined order), wherein each cycle comprises contacting the sample with the probes, allowing the probes to bind to their respective analytes (e.g., selected target nucleic acid sequences), and detecting a signal from the probes which have bound to the analytes, wherein in one or more cycles, the method comprises contacting the sample with an interfering agent that blocks binding (e.g., hybridization) and/or detection of probes for one or more analytes (e.g., selected target nucleic acid sequences), such that signals from the one or more analytes are not detected in those cycles (or are detected at a reduced level compared to signals in the absence of an interfering agent), thereby reducing signal crowding. In some embodiments, the bound (e.g., hybridized) probes and/or the detectable labels thereof are removed between cycles. In some embodiments, the interfering agent is contacted with the probes (e.g., mixed with the probes) before contacting the sample with the interfering agent and probes.

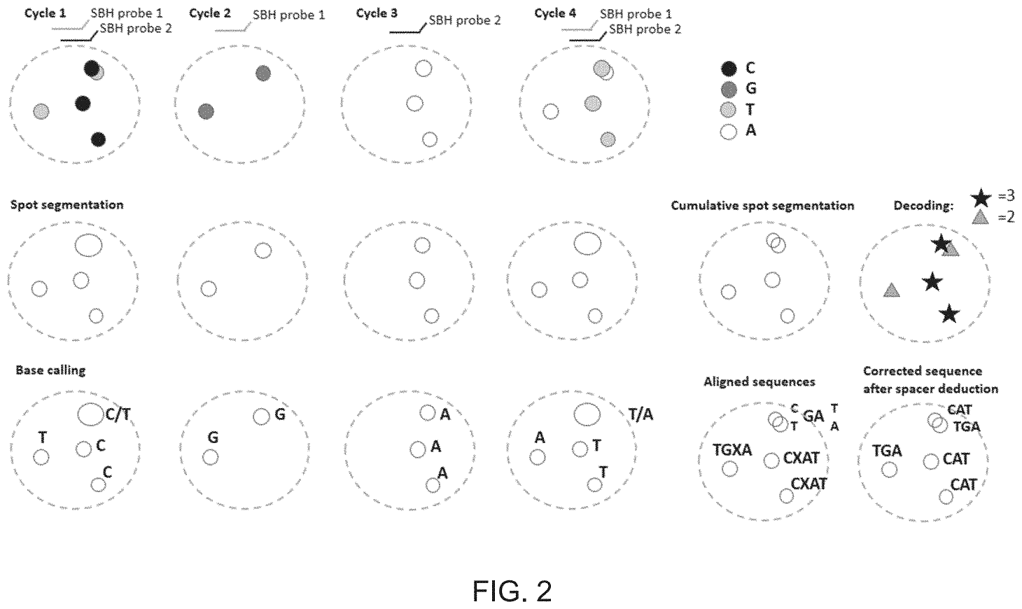

In some embodiments, provided herein is a method for analyzing a biological sample comprising a plurality of analytes, comprising detecting a plurality of optical signals in sequential cycles, wherein at least a subset of the plurality of optical signals detected at a location in the biological sample form an optical signature corresponding to an analyte of the plurality of analytes. In some embodiments, the detecting comprises in a first cycle (e.g., a non-sparse cycle), detecting a plurality of overlapping optical signals at a location, for instance, as shown in FIG. 2 where overlapping signals are detected in Cycle 1 (“C/T”) and Cycle 4 (“T/A”). In some embodiments, the detecting further comprises in a second cycle, detecting a first non-overlapping optical signal at the location, wherein the first non-overlapping optical signal is associated with a first analyte.

The second cycle can be a “sparse cycle” in which signals associated with the first analyte are detected, and “dark” for a second analyte—that is, signals associated with the second analyte are not detected. For instance, as shown in FIG. 2, a non-overlapping optical signal (“G”) associated with a first analyte (corresponding to “TGA”) is detected in Cycle 2 at a particular location, whereas a signal associated with a second analyte (e.g., corresponding to “CAT”) is not detected at the particular location in Cycle 2. In some embodiments, the detecting further comprises in a third cycle, detecting a second non-overlapping optical signal at the location, wherein the second non-overlapping optical signal is associated with a second analyte. The third cycle can be a “sparse cycle” in which signals associated with the second analyte are detected, and “dark” for the first analyte — that is, signals associated with the first analyte are not detected. For instance, as shown in FIG. 2, a non-overlapping optical signal (“A”) associated with a second analyte (corresponding to “CAT”) is detected in Cycle 3 at a particular location, whereas a signal associated with the first analyte (e.g., corresponding to “TGA”) is not detected at the particular location in Cycle 3.

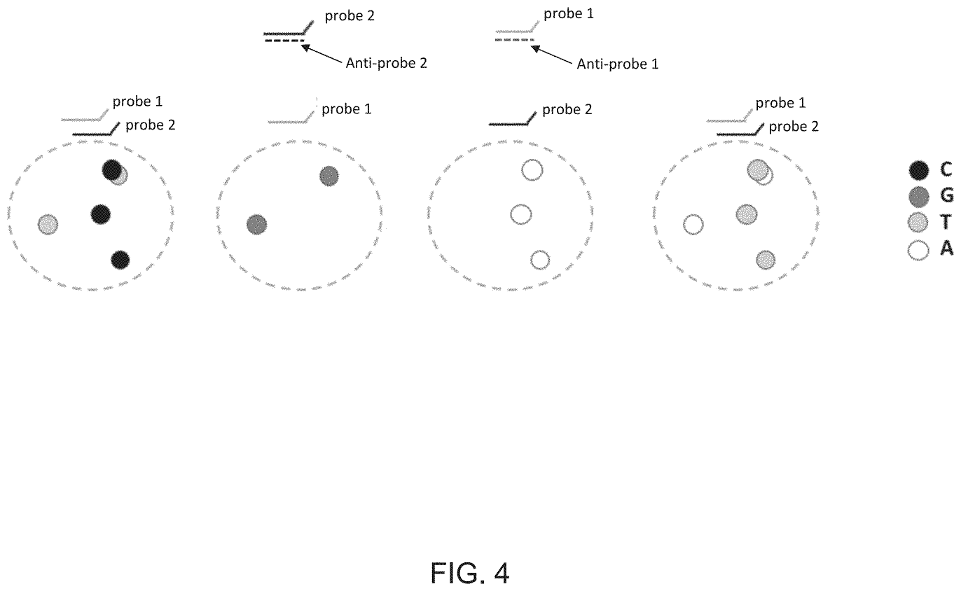

In another example shown in FIG. 4, the second cycle can be a “sparse cycle” in which signals associated with the first analyte are detected, and “dark” for a second analyte, wherein an interfering agent (Anti-probe 2) is included in Cycle 2 that blocks hybridization of a hybridization probe (probe 2) to the second analyte. In some embodiments, the detecting further comprises in a third cycle, detecting a second non-overlapping optical signal at the location, wherein the second non-overlapping optical signal is associated with a second analyte. The third cycle can be a “sparse cycle” in which signals associated with the second analyte are detected, and “dark” for the first analyte. For instance, as shown in FIG. 4, an interfering agent (Anti-probe 1) is included in Cycle 3 that blocks hybridization of a hybridization probe (probe 1) to the first analyte. In some instances, an interfering agent (Anti-probe 1) is included that blocks hybridization of a hybridization probe (probe 1) to a probe bound to the first analyte, or a probe bound to a product (e.g., an amplification product or extension product) of the first analyte. In some instances, an interfering agent (Anti-probe 1) is included that blocks hybridization of a hybridization probe (probe 1) to a product of a probe bound to the first analyte, such as a rolling circle amplification product of a circularized probe. In some embodiments, an interfering agent (Anti-probe 1) is included that blocks hybridization of a hybridization probe (probe 1) to a product of the first analyte, such as an amplification or extension product (e.g., cDNA).

In some embodiments, the method further comprises using an identifier for the first analyte and an identifier for the second analyte to associate a first overlapping optical signal of the plurality overlapping optical signals to the first analyte, and associate a second overlapping optical signal of the plurality overlapping optical signals to the second analyte, wherein the identifier for the first analyte is a first order of signal codes that identifies the first analyte, and the identifier for the second analyte is a second order of signal codes that identifies the second analyte. For instance, as shown in FIG. 2, the overlapping optical signal “T” of “C/T” in Cycle 1 can be associated with the first analyte (corresponding to “TGA”), and the overlapping optical signal “C” of “C/T” in Cycle 1 can be associated with the second analyte (corresponding to “CAT”). In some embodiments, the first order comprises signal codes that match an optical signature comprising the first overlapping optical signal and the first non-overlapping optical signal, and the second order comprises signal codes that match an optical signature comprising the second overlapping optical signal and the second non-overlapping optical signal, thereby identifying the first and second analytes at the location. For instance, as shown in FIG. 2, possible optical signatures at the overlapping positions (C/T)GA(T/A) can be compared to identifiers in a codebook or whitelist (e.g., one comprising identifiers TGA and

CAT) in order to identify the correct or most likely optical signatures corresponding to the first and second analytes, thereby identifying the first and second analytes at the location having overlapping signals.

In some embodiments, a plurality of analytes can be decoded (e.g., identified from other analytes) by contacting a sample containing or suspected of containing the analyte with detectably labeled probes that recognize the barcode sequences to produce probe binding patterns in sequential probe binding cycles, whereby patterns of detectably labeled probe binding can be compared to a list of known and/or allowed order of signal codes (e.g., identifiers) corresponding to the analytes (e.g., a codebook or “whitelist”). In some embodiments, the list of known and/or allowed order of signal codes (e.g., identifiers) corresponding to the analytes has not accounted for a sparse cycle or a dark cycle prior to decoding. In some embodiments, the identifiers in the list of known and/or allowed order of signal codes (e.g., identifiers) do not contain “0” or “X” bits indicating absence of signals (e.g., dark cycles). In some embodiments, all observed or recorded optical signatures, including all possible order of signal codes (e.g., identifiers) at locations having overlapping signals and/or at locations having non-overlapping signals (e.g., (C/T)GA(T/A), TGXA, or CXAT in FIG. 2), can be compared with the list of known and/or allowed order of signal codes (e.g., identifiers) for decoding, and for any given observed or recorded optical signature, it is not known a priori whether it contains any “0” or “X” bit or where any “0” or “X” bit is in the identifier. In some embodiments, the observed dark signals (e.g., lack of signal at a position) from a particular round is deduced and matched to the original known and/or allowed order of signal codes (e.g., identifiers).

In some embodiments, the inclusion or insertion of a “dark” signal code (e.g., a “0” or “X” bit) in an order of signal codes is not by design, e.g., it is not known a priori whether or not an observed or recorded optical signature comprises any “dark” signal code, or if present, where the “dark” signal code may be in the observed or recorded optical signature. In some embodiments, an algorithm can be used to compare a plurality of possible chains (e.g., all or substantially all possible chains of the observed or recorded optical signals or absence thereof) to a list of known and/or allowed order of signal codes (e.g., identifiers). In some embodiments, a match between a possible chain of optical signals or absence thereof and an identifier can be used to identify the corresponding analyte at a location in a sample, even when in one or more cycle the optical signal(s) associated with the analyte at the location may be overlapping with neighboring optical signal(s).

In some embodiments, decoding comprises compiling, processing, and analyzing a plurality of optical signals detected from sequential cycles at a location in a biological sample. In some aspects, a location comprising optical signals comprises optical signals from at least two neighboring spots (e.g., two analytes, such as two RCPs) that are close in proximity (e.g., optically overlapping). In some embodiments, a location may comprise two or more neighboring spots that are within 1 μm distance. In some embodiments, a location may comprise two or more neighboring spots that are within about 0.5 to 1 μm distance. In some cases, each location including all spots (e.g., overlapping spots) within that distance is observed and potential signal sequence chains are generated for this location. In some aspects, the optical signals from neighboring spots are detected and processed to determine which optical signals are associated with the first of the neighboring spots vs. the optical signals that are associated with the second of the neighboring spots. In some cases, an optical signal can be a presence of a signal (e.g., a detected signal in a channel) or an absence of a signal in the location. In some cases, the processing includes compiling the detected optical signals. In some cases, the processing includes generating a plurality of potential signal sequence chains with all possibilities of pairing the observed optical signals from various sequential cycles of detection. Then, the generated potential signal sequence chains can be compared to a list of known and/or allowed order of signal codes (e.g., of the identifiers) corresponding to the analytes (e.g., from a codebook or “whitelist”). In some cases, each of the potential signal code chains are assigned a probability (e.g. a likelihood) that the chain matches an identifier. In some embodiments, the analysis (e.g., processing of optical signals and generation of chain signals) can be performed using an algorithm. In some cases, this allows identification and matching, assigning or associating of the correct optical signals to the corresponding analyte (or to the other optical signals associated with the same analyte). In some embodiments, the processing allows any ambiguous optical signals (e.g., overlapping signals) to be associated or assigned to the corresponding analyte (or to the other optical signals associated with the same analyte).

In some embodiments, a probability of matching the identifier for the first or second analyte is assigned to each potential signal sequence chain of the plurality of potential signal sequence chains. In some embodiments, one or more modifications can be made to the potential signal sequence chains. In some cases, the potential signal sequence chain compared to the identifiers is processed to remove any absence of signals. In some cases, the potential signal sequence chain compared to the identifiers comprises detected signals and absence of signals.

In an exemplary workflow, RCA products (RCP) at a location in a biological sample can be identified from individual channels in different cycles. For each RCP, its nearest neighbors within 1 μm distance across all images can be identified, and only the closest neighbor from each image cycle is kept. In this example, a sequence of channels can be constructed into a “chained” event (e.g., a potential signal sequence chain) that includes certain detected signals and uncertain signals(?). For example, an object (e.g., a location) can have the chained sequence: cycle1channel4 (c1c4)-c2c1-c3?-c4c2-c5?-c6c3). In some cases, a detected signal is considered certain because only one signal (e.g. one channel or color) is detected during that cycle (e.g., c2c1). In some cases, a detected signal is considered uncertain because more than one signal (e.g., two channels or colors) are detected during that cycle (e.g., c3?). During the generation of the potential signal sequence chains, all potential signal sequence chain possibilities can be generated and kept. In some cases, the generation of all the potential signal sequence chains are performed by an automated algorithm. The automated algorithm can then compare the potential signal sequence chains with all possibilities to all existing barcode sequences (e.g. identifiers in a codebook). The algorithm may also compare and determine the likelihood of a match between each potential signal sequence chain to existing barcodes. Then, the highest likely match can be determined. In some cases, the dark spacers are deduced from the signal sequence chain resulting in the final barcode sequence that matches the coding sequence with % of probability. In some aspects, this enables the disentangling of ambiguous chains that are the result of 2 optically overlapping objects (e.g., RCPs) with different barcode sequence, which can be distinguished as two separate barcodes. The described sparse labelling approach may allow the identification of more very closely localized RCPs and more accurate quantification.

In some embodiments, during the process of generating the potential signal sequence chains and matching to the identifiers, it can allow association of: 1) ambiguous detected signals comprising two colors or from two channels (e.g., R/G) at a location comprising two neighboring spots, and/or 2) a detected signal and a deduced dark signal, to the first of the neighboring spots vs. the second of the neighboring spots.

In some examples, the method can be applied to two genes at an optically overlapping location in a biological sample which can be detected from individual channels in different cycles as outlined below in Table 1. In step 1, the identifiers (an order of signal codes) of the two exemplary genes are provided. In step 2, dark cycles can be inserted into the order of signal codes as denoted by the (X's) by omitting detection probes or blocking detection probes from binding the target analyte (directly or indirectly via an interfering agent such as an intermediate probe). In some cases, it is not known a priori whether or where (in which cycle) any dark cycles would be inserted in the identifier. In step 3, the optical signals from each channel detected in each cycle is provided, including where overlapping signals are detected (e.g., R/G, detected in both red and green channels) or where no overlap is detected (in some examples due to an inserted dark round, e.g., X/B, signal detected only in the blue channel). In step 4, an algorithm may be used to automatically generate all possible signal sequence chains from the observed signals from step 3. Only a portion of all possible signal sequence chains are provided for illustrative purposes. In some embodiments, the signal sequence chains can be compared to the identifiers (e.g., Step 1) to assign a likelihood of a match to the identifier. In some cases, in the comparison of potential signal sequence chains to identifiers, a percentage, possibility, or likelihood of a match is assigned to the potential signal sequence chain. In some embodiments, the signal sequence chains can be collapsed to remove the dark cycles (X's).

TABLE 1
Exemplary Decoding with Two Genes

| Step | Gene 1: Calb2 | Gene 2 : chodl |
|---|---|---|
| 1. Identifiers (e.g., | GGRB | GBRG |
| signal code) | | |
| G = green, R = red, | | |
| B = blue | | |
| 2. Signal code | GXGXRB | GBXRGX |
| with dark | | |
| (X) inserted | | |

| | |
|---|---|
| 3. Optical | Cycles: 1-2-3-4-5-6 |
| signals detected | G/G-X/B-X/G- |
| including dark (X) | X/R-R/G-B/X |
| 4. Potential signal | G-X-G-R-G-X |
| sequence | G-B-X-R-G-X -> match chodl |
| chains generated | (collapsed GBRG) |
| | G-X-X-R-G-B |
| | G-X-X-R-R-X |
| | G-X-X-R-R-B |
| | G-B-X-X-G-X |
| | G-B-G-X-G-X |
| | G-X-G-X-R-B -> match calb2 |
| | (collapsed GGRB) . . . many more |
| | potential signal sequence chains |

In some embodiments, sparse labelling as described herein can be combined with an intentional design for sparsely decoding analytes where it is known a priori that the detection of signals for an analyte contains one or more dark cycles, or “0” or “X” bit and where the “0” or “X” bit is in the identifier (e.g., codebook). In some aspects, decoding for the intentionally inserted and known dark cycle may not include deducing whether a dark cycle exists, where a dark cycle is, and/or may not include generation of potential signal sequence chains.

As described above, a dark cycle for a given analyte may be achieved by omitting the probe for the given analyte in that cycle, or by contacting the sample with an interfering agent for that analyte or probe. In some embodiments, the interfering agent can block hybridization of the probe to the analyte (e.g., by hybridizing to the target-binding region of the probe as shown in FIG. 4, or by hybridizing to the target sequence of the analyte). In some embodiments, the interfering agent can displace the probe from the analyte. In some embodiments, the interfering agent can block detection of a signal from the probe (e.g., interfere with detectably labeled probes that bind the hybridization probe or the interfering agent can comprise a quencher moiety that blocks detection of the signal). In some embodiments, the interfering agent can interfere with hybridization of a detection oligonucleotide to the probe.

In some embodiments, provided herein is a method for nucleic acid sequence detection, comprising: (a) contacting a sample with multiple sets of probes (e.g., hybridization probes) in sequential cycles, wherein each set is specific for a corresponding target nucleic acid sequence and each set comprises multiple probes that (i) hybridize to a particular nucleic acid sequence and (ii) are directly or indirectly labelled with a detectable label which may be the same or different for probes in the same probe set, wherein the sequential cycles comprise contacting the sample with probes within a set in a pre-determined order which corresponds to a signal code sequence for the corresponding target nucleic acid sequence, wherein the sequential cycles comprise one or more de-crowded cycles in which only probes for a subset of the target nucleic acid sequences are contacted with the sample and are detected, whereby probes for the remaining target nucleic acid sequences are not contacted with the sample and thereby not detected; and (b) obtaining a signal code sequence for each target nucleic acid sequence, wherein the signal code sequence comprises signal codes corresponding to the signals or absence thereof from detectable labels for probes in the sequential cycles, thereby detecting the target nucleic acid sequences in the sample. In some embodiments, a spacer signal code is provided to a particular target nucleic acid sequence in a particular de-crowded cycle in which probes for that particular target nucleic acid sequence are not contacted with the sample in that particular de-crowded cycle. In some embodiments, one or more spacer signal codes are removed from the signal code sequence for each target nucleic acid sequence to provide a corrected signal code sequence which uniquely identifies that target nucleic acid sequence among the target nucleic acid sequences.

In some embodiments, provided herein is a method for nucleic acid sequence detection, comprising: (a) contacting a sample with multiple sets of probes (e.g., hybridization probes) in sequential cycles, wherein each set is specific for a corresponding target nucleic acid sequence and each set comprises multiple probes that (i) hybridize to a particular nucleic acid sequence and (ii) are directly or indirectly labelled with a detectable label which may be the same or different for probes in the same probe set, wherein the sequential cycles comprise contacting the sample with probes within a set in a pre-determined order which corresponds to a signal code sequence for the corresponding target nucleic acid sequence, wherein the sequential cycles comprise one or more de-crowded cycles in which the sample is contacted with interfering agents for one or more selected target nucleic acid sequences, whereby signals associated with the selected target nucleic acid sequences are not detected or are detected at a reduced level compared to signals detected in the absence of the interfering agent; and (b) obtaining a signal code sequence for each target nucleic acid sequence, wherein the signal code sequence comprises signal codes corresponding to the signals or absence thereof from detectable labels for probes in the sequential cycles, thereby detecting the target nucleic acid sequences in the sample. In some embodiments, a spacer signal code is provided to a particular target nucleic acid sequence in a particular de-crowded cycle in which an interfering agent for that particular target nucleic acid sequence is contacted with the sample in that particular de-crowded cycle. In some embodiments, one or more spacer signal codes are removed from the signal code sequence for each target nucleic acid sequence to provide a corrected signal code sequence which uniquely identifies that target nucleic acid sequence among the target nucleic acid sequences.

In any of the embodiments herein, instead of or in combination with omitting a detectable probe (e.g., a fluorescently labeled probe and/or an intermediate probe that hybridizes to both the fluorescently labeled probe and a target nucleic acid, also referred to as a “hybridization probe” herein), an interfering agent such as a blocking probe, also termed herein “antidote probe” in some examples, may be used to prevent signals from being generated and/or detected or reduce the amplitude of signals indicative of certain (e.g., selected) target nucleic acid sequences (e.g., reporter sequence(s) or barcode(s) associated with a given analyte). In some aspects, an antidote probe can be viewed as a blocking probe, which blocks, inhibits, or prevents a hybridization probe from functioning to detect its target and/or a detection probe from functioning to detect the hybridization probe. In some embodiments, this may be achieved by blocking or reducing the binding of the hybridization probe to its target (e.g., by allowing an antidote probe to hybridize to the hybridization probe or by allowing an antidote probe to hybridize to the target). In some embodiments, the method comprises blocking or reducing the binding of one or more detection probes (e.g., fluorescently labelled oligos) to the hybridization probe while binding between the hybridization probe and its target is not blocked or reduced. In some embodiments, the method comprises allowing an antidote probe to hybridize to a hybridization probe at a sequence outside of the target-binding sequence of the hybridization probe (e.g., an overhang of the hybridization probe hybridized to its target). In some aspects, an antidote probe may be termed a “silencer probe,” as it can be seen to function to silence a signal indicative of an analyte, e.g., a signal from a hybridization probe corresponding to the analyte. Exemplary interfering agents/antidote probes are described in more detail in below.

In one aspect, provided herein is a method of detecting multiple target nucleic acid sequences in a sample (e.g., multiple reporter sequences or barcodes associated with analytes in a sample), wherein the target nucleic acid sequences are detected by hybridization probes which hybridize to the target nucleic acid sequences and provide detectable signals that allow the target nucleic acid sequences to be identified and detected, said method comprising: (a) providing a plurality of hybridization probes comprising different hybridization probes each specific for a different target nucleic acid sequence, wherein each hybridization probe has a recognition sequence complementary to a sequence within its target nucleic acid sequence, and is capable of giving rise to a signal by means of which it can be detected; (b) further providing at least one antidote probe for at least one selected hybridization probe specific for a selected target nucleic acid sequence; (c) contacting the sample with the plurality of hybridization probes of (a) and the antidote probe(s) of (b), allowing the antidote probe(s) to hybridize to the selected hybridization probe(s) or the selected target nucleic acid sequence(s), and allowing the hybridization probes to hybridize to target nucleic acid sequences that are present in the sample; (d) detecting a signal from each hybridization probe which has hybridized to its target sequence, wherein signals are not detected from the selected target nucleic acid sequences of (b); (e) identifying target nucleic acid sequences from the signals detected in step (d), and thereby detecting those target nucleic acid sequences in the sample.

In some embodiments, provided herein is a method for nucleic acid sequence detection, comprising: (a) contacting a sample with a first probe set and a second probe set in sequential cycles, wherein: the first probe set hybridizing to a first target nucleic acid sequence, wherein the first probe set comprises P11, P12, and P13, the second probe set hybridizing to a second target nucleic acid sequence different from the first target nucleic acid sequence, wherein the second probe set comprises P21, P22, and P23, each probe is directly or indirectly labelled with a detectable label which may be the same or different for probes in the same probe set, and the sequential cycles comprise, in any order, (i) contacting the sample with P11 and P21, (ii) contacting the sample with P12; (iii) contacting the sample with P22, and (iv) contacting the sample with P13 and P23; and (b) obtaining a signal code sequence for each target nucleic acid sequence, wherein the signal code sequence comprises signal codes corresponding to the signals or absence thereof from detectable labels for probes in cycles (i)-(iv), thereby detecting the first and second target nucleic acid sequences in the sample.

In some embodiments, provided herein is a method for nucleic acid sequence detection, comprising: (a) contacting a sample with a first probe set and a second probe set in sequential cycles, wherein: the first probe set hybridizing to a first target nucleic acid sequence, wherein the first probe set comprises P11, P12, P13, and P14, the second probe set hybridizing to a second target nucleic acid sequence different from the first target nucleic acid sequence, wherein the second probe set comprises P21, P22, P23, and P24, each probe is directly or indirectly labelled with a detectable label which may be the same or different for probes in the same probe set, and the sequential cycles comprise, in any order, (i) contacting the sample with P11 and P21, (ii) contacting the sample with P12, P22, and an antidote probe that blocks interaction between P22 and the second target nucleic acid sequence (e.g., the antidote probe can hybridize to P22 or the second target nucleic acid sequence); (iii) contacting the sample with P13, P23, and an antidote probe that blocks interaction between P13 and the first target nucleic acid sequence (e.g., the antidote probe can hybridize to P13 or the first target nucleic acid sequence); and (iv) contacting the sample with P14 and P24; and (b) obtaining a signal code sequence for each target nucleic acid sequence, wherein the signal code sequence comprises signal codes corresponding to the signals or absence thereof from detectable labels for probes in cycles (i)-(iv), thereby detecting the first and second target nucleic acid sequences in the sample.

In some embodiments, the signal code sequence uniquely identifies the corresponding target nucleic acid sequence. In some embodiments, probes in the same probe set can be used in a pre-determined order in cycles (i)-(iv) which corresponds to the signal code sequence for the target nucleic acid sequence. In some embodiments, a signal from the detectable label for probes in the second set is not detected in cycle (ii), thereby providing a spacer signal code corresponding to cycle (ii) in the signal code sequence for the second target nucleic acid sequence, and a signal from the detectable label for probes in the first set is not detected in cycle (iii), thereby providing a spacer signal code corresponding to cycle (iii) in the signal code sequence for the first target nucleic acid sequence. In some embodiments, the spacer signal codes can be removed from the signal code sequences. In some embodiments, P11, P12, and

P13 can comprise a common recognition sequence complementary to a barcode sequence in the first target nucleic acid sequence, and P21, P22, and P23 can comprise a common recognition sequence complementary to a barcode sequence in the second target nucleic acid sequence.

In some embodiments, described herein is a method of localized detection of multiple target nucleic acid sequences in a sample, wherein each target nucleic acid sequence is detected using a circularizable probe specific for said target sequence which is circularized upon hybridization to the target sequence and is amplified by rolling circle amplification (RCA) to produce a rolling circle product (RCP), each circularizable probe comprising a different barcode sequence specific for a different target nucleic acid sequence, and each RCP containing multiple complementary copies of the barcode sequence, wherein the barcode sequence is decoded in multiple sequential decoding cycles each using hybridization probes which hybridize to the complementary copies of the barcode sequence in a RCP and provide detectable signals which together yield a unique signal code sequence which identifies the target nucleic acid sequence, and wherein signal crowding from said hybridization probes is reduced, characterized in that the method comprises: (a) providing multiple sets of hybridization probes, wherein each set is specific for a different target nucleic acid sequence and each set comprises multiple hybridization probes, each hybridization probe in a set (i) comprising a common recognition sequence complementary to the barcode sequence for that nucleic acid sequence and (ii) being directly or indirectly labelled with a detectable label which gives rise to a given signal, which may be the same or different, and wherein the hybridization probes within a set are designated for use in a pre-determined order which corresponds to the signal code sequence for that target nucleic acid sequence; (b) performing multiple cycles of decoding using the hybridization probes of each set in their pre-determined order, wherein each cycle comprises contacting the RCPs with hybridization probes, allowing the hybridization probes to hybridize to their respective RCPs, and detecting a signal from the hybridization probes which have hybridized to the RCPs, wherein in at least some cycles hybridization probes for one or more target nucleic acid sequences, or the detectable labels thereof, are omitted, such that signals from the target nucleic acid sequences are not detected in those cycles, thereby reducing signal crowding, and wherein hybridized hybridization probes are removed between cycles; (c) analyzing the collective signals detected in all the cycles of step (b) to identify signals which correspond to signal codes within a signal code sequence, and determining a signal code sequence therefrom, thereby to identify and detect the target nucleic acid sequences, wherein each signal code sequence is composed only of detected signals from the detectable labels (e.g., determined by the presence of a label).

In some embodiments, the omission of hybridization probes (or the detectable labels thereof) for one or more of the target nucleic acid sequences of (b) means that signals are not detected from these target nucleic acid sequences, thus reducing signal crowding and allowing signals from other target nucleic acid sequences to be resolved.

In some embodiments, absence of signal may be recorded and the corresponding “dark” (or spacer) signal code may be removed so that it does not contribute to the corrected signal code sequence. In some aspects, in a dark cycle in which a hybridization probe is omitted for a first target, a signal indicative of the first target is not detected, and that dark cycle is not scored as part of the detection of the first target. However, that same dark cycle for the first target may be a non-dark cycle for a second target, and one or more signal spots indicative of the second target may be detected and resolved from other spots in that cycle, including signal spots that might otherwise overlap or become crowded out by signal spot(s) indicative of the first target, if signal spots for both the first and second targets were to be detected in the same cycle.

In some embodiments, also provided herein is a method for reducing signal crowding in the detection of nucleic acids, involving the so-called “sparse labelling” method. In some embodiments, the problem of signal crowding is addressed by detecting the necessary signals from the hybridization probes over an increased number of decoding cycles, including some cycles in which given target(s) are not detected, e.g., blank or dark cycles, thereby reducing the number of signals which are generated and detected at any single time (or in any single cycle). In some aspects, the total number of cycles is greater than the number of positions in an signal code sequence (e.g., first or second order of signal codes) of an identifier. In some aspects, this method is accordingly applicable to a sequential combinatorial labelling scheme, and in particular to a sequential SBH method, in which a sequential series of SBH cycles are performed using hybridization probes to decode a barcode in a series of cycles.

In some embodiments, a sequential decoding scheme involves detecting repeated signals from a given target in multiple cycles, and the target may in the same position in the sample in the different cycles. In some embodiments, a method disclosed herein comprises the localized detection of the target nucleic acid sequences. This is discussed and defined further below. In some embodiments, the target nucleic acid sequence is present at a fixed or defined location in the sample, and is detected at that location. The target nucleic acid sequence may be localized by virtue of being present in situ at its native location in the sample (e.g. a cell or tissue sample), or of being attached or otherwise localized to a target analyte which is present in situ at its native location in the sample. The target nucleic acid sequence may be immobilized in the sample. Thus, a target analyte or a target nucleic acid sequence may be artificially immobilized in the sample, e.g. fixed on or to a solid surface, or bound to an immobilizing moiety provided on a solid surface.

In some embodiments, provided herein is a method comprising detecting target nucleic acid sequences by padlock probes, or circularizable probes or probe sets more generally, wherein the padlock probe is detected by detecting a rolling circle amplification (RCA) product of the padlock probe.

In some embodiments, each target nucleic acid sequence in the sample is detected using a circularizable probe specific for that target sequence which is circularized upon hybridization to the target sequence and is amplified by rolling circle amplification (RCA) to produce a rolling circle product (RCP). In some embodiments, each circularizable probe comprises a different barcode sequence specific for a different target nucleic acid sequence, and therefore each RCP contains multiple complementary copies of the barcode sequence.

In some embodiments, the barcode sequence is decoded in multiple sequential decoding cycles. In some embodiments, the barcode sequence corresponds to a unique signal code sequence which is specific to the target nucleic acid sequence. In some embodiments, each decoding cycle uses hybridization probes which hybridize to the complementary copies of the barcode sequence in the RCP and provide detectable signals, wherein each signal corresponds to an individual signal code, and the signal codes together yield the unique signal code sequence which identifies the target nucleic acid sequence.

The circularizable probe may be of any design known in the art which, when circularized, is suitable for amplification by RCA. In some embodiments, a “circularizable” probe can take the form of a linear molecule having ligatable ends which may circularized by ligating the ends together directly or indirectly, e.g., to each other, or to the respective ends of an intervening (“gap”) oligonucleotide, or to an end generated by extension of a 3′ end of the probe (so-called “gap-filling extension”) to form a circularized probe hybridized to the target nucleic acid sequence. In some embodiments, the circularizable probe may be a padlock probe. The circularizable probe may also be provided in two or more parts, namely two or more molecules (e.g. oligonucleotides) which may be ligated together to form a circle. The circularizable probe is circularized by ligation prior to the RCA reaction. This ligation may be templated using a ligation template, which may be the target nucleic acid sequence. The circularizable probe will comprise at its respective 3′ and 5′ ends, regions of complementarity to corresponding cognate complementary regions (or binding sites) in the target nucleic acid sequence, which may be adjacent where the ends are directly ligated to each other, or non-adjacent, with an intervening “gap” sequence, where indirect ligation is to take place.

In some embodiments, the sequence of the circularizable probe forms the template for the RCA reaction, and thus it can be seen that the RCP will contain repeated copies of the complement of a barcode sequence provided in the circularizable probe. The sequence present in the circularizable probe which is capable of hybridizing to the target nucleic acid sequence may act as a barcode sequence. In some embodiments, the barcode sequence may be a synthetic or artificial sequence which is present in the portion of the circularizable probe which does not hybridize to the target nucleic acid sequence. The circularizable probe comprises a barcode sequence for the target nucleic acid sequence to which it is capable of hybridizing. The barcode sequence is then copied, as a complementary sequence, into the RCP. The term “barcode sequence” can therefore encompass both a barcode sequence present in the RCP and its complement (more particularly reverse complement) present in the circularizable probe. Accordingly, a “barcode sequence” as referred to herein can include the complementary sequence.

In some embodiments, a method disclosed herein comprises a step of providing multiple sets of hybridization probes, wherein each set is specific for a different target nucleic acid sequence and each set comprises multiple hybridization probes. In some embodiments, each hybridization probe in a set comprises a common recognition sequence complementary to the barcode sequence for that target nucleic acid sequence, such that all of the hybridization probes in the set are capable of hybridizing to the barcode sequence. In some embodiments, each hybridization probe in a set is directly or indirectly labelled with a detectable label which gives rise to a given signal, which may be the same or different, and the hybridization probes within a set are designated for use in a pre-determined order, which corresponds to the signal code sequence for that target nucleic acid sequence. In some embodiments, in the “sparse labelling” detection method, the hybridization probes are directly or indirectly labelled with a detectable label. In some embodiments, hybridization probes which lack a detectable label and which therefore signal via the absence of a detectable signal are not used in the “sparse labelling” detection method. In other embodiments, one or more of the hybridization probes are not detectably labelled. For example, the one or more hybridization probes may lack sequences for hybridization to and detection by one or more reporter or detection probes (e.g., fluorescently labelled detection oligos) and do not provide a detectable signal in the sample (e.g., after washing away unbound probes) even when contacted with the reporter or detection probes in a cycle. In an embodiment, the hybridization probes provide a signal indirectly via a reporter domain which is not complementary to the target nucleic acid sequence, but which comprises a binding site for a reporter probe which comprises a detectable label.

In some embodiments, hybridization probes in different sets have different recognition sequences, such that they are capable of hybridizing to the barcode sequences for different target nucleic acid sequences. In some embodiments, each set of hybridization probes is designed to bind to a different target sequence, in particular a different barcode sequence.

In some embodiments, the detection method comprises a step (b) of performing multiple cycles of decoding using the hybridization probes of each set in their pre-determined order, wherein each cycle comprises contacting the RCPs with hybridization probes, allowing the hybridization probes to hybridize to their respective RCPs, and detecting a signal from the hybridization probes which have hybridized to the RCPs. The hybridization probes that have hybridized to their respective RCPs are removed between cycles, such that the hybridization probes from the subsequent cycle can hybridize to the RCPs in their place.

In some embodiments, in order to reduce signal crowding, in at least one cycle the hybridization probes for one or more target nucleic acid sequences, and/or the detectable labels thereof (e.g. the reporter probes), are omitted, such that signals from the target nucleic acid sequences are not detected in those cycles. In some embodiments, this reduces the number of signals that are generated, and thus allows for signals from other target nucleic acid sequences to be detected and resolved.

In an embodiment, the hybridization probes for one or more target nucleic acid sequences are omitted. In this case, there is no hybridization probe to hybridize to the complement of the barcode sequence in the RCP corresponding to that target nucleic acid sequence, and thus no signal can be detected for that target nucleic acid sequence. Alternatively, the reporter probes for the hybridization probes for one or more target nucleic acid sequences may be omitted. In this case, although the hybridization probes may hybridize to the RCPs in respect of the target nucleic acid sequences, the reporter probes will not be present, and thus no detectable signal will be generated. The end result in terms of signal detection is therefore the same, whether the hybridization probes or the reporter probes are omitted.

In some embodiments, a target nucleic acid sequence may be selected based on their relative abundance in the sample, for example sequences which are present at high levels (high abundance), and which do or may lead to signal crowding. In some embodiments, a target nucleic acid sequence may be designated randomly (e.g., not based on knowledge or suspicion that the particular target nucleic acid sequence may contribute to signal crowding), for example, as part of a combinatorial scheme. In some embodiments, a target nucleic acid sequence that is known to be highly expressed is not detected in a plurality of cycles whole other target sequences are detected e.g., by omitting the hybridization probes for the highly expressed target nucleic acid or the reporter probes for the hybridization probes for the highly expressed target nucleic acid. In some cases, a target sequence that is known to be highly expressed is detected separately and individually from other target sequences.

In some embodiments, the cycle in which the hybridization probes for one or more selected or designated target nucleic acid sequences (or the detectable labels thereof) are omitted or not detected may be referred to as a dark cycle in respect of the one or more selected or designated target nucleic acid sequences. The dark cycle, which may also be referred to a “blank cycle”, acts as a spacer, or space holder, in the decoding of that barcode; it is does not contribute to the signal code sequence which is derived to the barcode.

The number of dark cycles, both in total, and for an individual barcode may be varied. It is not necessary for there to be a dark cycle for each barcode, although in an embodiment at least one dark cycle is included for each barcode to be read. In another embodiment, a dark cycle is included only for selected or designated barcodes (e.g., selected or designated targets), for example for targets which are present in increased amounts, or which are known or determined to cause signal crowding. Similarly, it is not necessary for a dark cycle to be included in each cycle of decoding (e.g. in each round of cycles). In other words, it is not necessary that in each cycle a hybridization probe for a selected or designated target, or a detectable label thereof, is omitted, as long as in the multiple cycles which are performed there are included at least some dark cycles, for at least some target nucleic acid sequences. Accordingly, in an embodiment all the hybridization probes for one or more decoding cycles can be labelled, as long as in the method as a whole there is at least one decoding cycle in which a hybridization probe or detectable label thereof is omitted.

In some embodiments, a cycle wherein the hybridization probes for one or more selected or designated target nucleic acid sequences (or the detectable labels thereof) are omitted may occur once during decoding process. Alternatively put, there may be one dark cycle in the decoding of the barcode sequence.

Depending on the degree to which signal crowding occurs, the number of dark cycles may be varied. If it is desired to detect a large number of different target nucleic acid sequences in a given sample, and thus it is necessary to detect a large number of signals simultaneously, more dark cycles may be employed to reduce the number of signals which are generated in any one cycle. Accordingly, in some embodiments, there may be more than one, such as 2, 3, 4, 5 or more dark cycles in the decoding of the barcode sequence. It will be understood that where multiple dark cycles are used, the one or more selected or designated target nucleic acid sequences for which the hybridization probes (or the detectable labels thereof) are omitted may vary between decoding cycles. In some embodiments, for a plurality of analytes in the sample, the same number of dark cycles are used. For example, decoding a first and second analyte comprises the incorporation of the same number of dark cycles for each analyte.

In some embodiments, at least one dark cycle is included per 3 cycles, at least one dark cycle is included per 4 cycles, at least one dark cycle is included per 5 cycles, at least one dark cycle is included per 6 cycles, at least one dark cycle is included per 7 cycles, or at least one dark cycle is included per 8 cycles. In some examples, at least two dark cycles are included per 6 cycles, at least two dark cycles are included per 7 cycles, at least two dark cycles are included per 8 cycles, at least two dark cycles are included per 9 cycles, or at least two dark cycles are included per 10 cycles. In some examples, at least three dark cycles are included per 8 cycles, at least three dark cycles are included per 9 cycles, at least three dark cycles are included per 10 cycles, at least three dark cycles are included per 11 cycles, or at least three dark cycles are included per 12 cycles.

For example, if the method is applied to a sample comprising three different target nucleic acid sequences, the hybridization probes for the first target nucleic acid sequence may be omitted in the first cycle, such that the RCPs are only contacted with the hybridization probes for the second and third target nucleic acid sequences. Subsequently, in the second decoding cycle, the hybridization probes for the second target nucleic acid sequence may be omitted, such that the RCPs are only contacted with the hybridization probes for the first and third target nucleic acid sequences.

In some embodiments, the number of target nucleic acid sequences for which the hybridization probes (or the detectable labels thereof) are omitted in a dark cycle may similarly be varied. In some embodiments, only the hybridization probes (or the detectable labels thereof) for a single target nucleic acid sequence may be omitted. Alternatively, if there are multiple target nucleic acid sequences for which a large number of signals are being generated, such that signals in respect of other target nucleic acid sequences cannot be properly detected and resolved, then the hybridization probes (or the detectable labels thereof) for multiple target nucleic acid sequences may be omitted. It will be clear that the hybridization probes (or the detectable labels thereof) for any number of target nucleic acid sequences may be omitted, provided that the RCPs are contacted with a hybridization probe for at least one target nucleic acid sequence. Alternatively put, each decoding cycle must comprise a hybridization probe for at least one target nucleic acid sequence.

In some embodiments, increasing the number of dark cycles, or increasing the number of hybridization probes (or detectable labels) which are omitted in each dark cycle, increases the resolution and dynamic range of the detection method (e.g. will increase the extent to which signal crowding is reduced).

In a particular embodiment, every decoding cycle may be a dark cycle for at least one target nucleic acid sequence. That is to say that in each cycle of decoding, the hybridization probes for one or more selected or designated target nucleic acid sequences (or the detectable labels thereof) are omitted in each decoding cycle. Again, it will be clear that the selected or designated target nucleic acid sequences for which the hybridization probes (or the detectable labels thereof) are omitted may vary between decoding cycles. Alternatively put, the decoding may be arranged such that no decoding cycle involves the use of a hybridization probe for each target nucleic acid sequence.

In some embodiments, the signals detected in the sequential decoding cycles are analyzed in a step (c) of the detection method in order to identify the individual signal codes which correspond to the detected signals, and therefore the signal code sequences for the target nucleic acid sequences. The signal code sequences in turn allow the target nucleic acid sequences to be identified and therefore detected within the sample. In the analysis, the dark cycles can be deduced from the signals that are detected, and the identified signal codes. The dark cycles can be removed from the signal codes identified for a given barcode (target sequence), and the remaining signal code sequence can be used to identify the barcode, and hence the target nucleic acid sequence. The analysis step thus comprises analyzing the signals detected from the multiple decoding cycles, identifying signal codes, deducing the cycles in which for a given selected or designated target nucleic acid sequence a hybridization probe or detectable label thereof was omitted, removing the said deduced cycles from the detected signal (and hence signal codes), and deriving a signal code sequence from the remaining identified signal codes. Thus, by analysis of the detected signals, the dark cycles for each barcode can be identified, and for each barcode a signal code can be extracted from each cycle, and a signal code sequence derived which matches a known signal code sequence, thereby to identify that signal code sequence. Software or algorithms for performing such an analysis, for example by image analysis of fluorescent signals, is available or can readily be modified for use by the skilled person.

In some embodiments, a signal code sequence is composed only of detected signals from the detectable labels. In some embodiments, hybridization probes which lack a detectable label and therefore signal using the absence of a signal are not used in a “sparse labelling” method disclosed herein. In some embodiments, the signal code sequences which are assigned to the target nucleic acid sequences only comprise actively detected signals, e.g., they do not comprise at any point the absence of a signal. In some embodiments of the “sparse labelling” method, the absence of a signal in respect of a particular target nucleic acid sequence may be observed, but this will be indicative of a dark cycle in respect of the target nucleic acid sequence in question, where the hybridization probe for that target nucleic acid sequence has been omitted, which is not included in the signal code sequence. In some embodiments, the individual signals within the signal code sequence are detected (e.g., the presence or absence of any dark cycles, and the sequence thereof) does not alter the identity of the signal code sequence. In some embodiments, the signal code sequences which are assigned to the target nucleic acid sequences are unique (e.g., no two signal code sequences are identical), even when dark cycles are not included in the signal code sequence.

In some embodiments, hybridization probes which lack a detectable label may be used in a “sparse labelling” method disclosed herein; instead of omitting hybridization probes for a given target in a cycle, hybridization probes which lack a detectable label may be used. For instance, in examples using the first probe set (comprising P11, P12, and P13) for the first nucleic acid target sequence and the second probe set (comprising P21, P22, and P23) for the second nucleic acid target sequence in sequential cycles comprising, in any order, cycles (i) to (iv), the probe/cycle configurations can include:

| | Cycle i | Cycle ii | Cycle iii | Cycle iv |
|---|---|---|---|---|
| Target | Probes | Probe(s) | Probe(s) | Probes |
| Target | P11 | P12 | No probe | P13 |
| 1 | | | for Target 1 | |
| Target | P21 | No probe | P22 | P23 |
| 2 | | for Target 2 | | |

In some embodiments, Cycle ii may include one or more probes for the second nucleic acid target, and/or Cycle iii may include one or more probes for the first nucleic acid target. In some embodiments, the one or more probes for the first or second nucleic acid target may lack a detectable label or comprises a detectable label that is not detected in the same cycle as the probe(s) for the second or first nucleic acid target, respectively. For example, hybridization probes P22′ and P12′ may be included in Cycle ii and Cycle iii, respectively, where probe P22′ may lack a detectable label that can be detected by a detection probe in Cycle ii and therefore does not give rise to a signal when P12 (indicative of the first target) is detected in Cycle ii. Similarly, probe P12′ may lack a detectable label that can be detected by a detection probe in Cycle iii and therefore does not give rise to a signal when P22 (indicative of the second target) is detected in Cycle iii:

| | Cycle i | Cycle ii | Cycle iii | Cycle iv |
|---|---|---|---|---|
| Target | Probes | Probe(s) | Probe(s) | Probes |
| Target 1 | P11 | P12 | P12′ | P13 |
| Target 2 | P21 | P22′ | P22 | P23 |

In some embodiments, if a given target nucleic acid sequence is assigned the signal code R-G-B (wherein R represents a red fluorescent label, G represents a green fluorescent label, and B represents a blue fluorescent label), the individual signal codes within this signal code sequence may be detected in only 3 decoding cycles (one for each signal code), or there may be one or more dark cycles. In the first decoding cycle, for example, a hybridization probe comprising a red fluorescent label may be used, and thus a red signal may be detected. In the second decoding cycle, the hybridization probes for the target nucleic acid sequence in question may be omitted, such that no signal is detected. At this point, the only signal code which has been identified in respect of the target nucleic acid sequence is R. The absence of the signal which was observed during the second decoding cycle (whilst signals were detected in respect of other target nucleic acid sequences within the sample) does not form part of the signal code sequence. The subsequent decoding cycles, possibly including one or more additional dark cycles, will in due course lead to the detection of green and blue signals, and therefore to the identification of the signal code sequence for the target nucleic acid sequence. Thus, the absence of signal in a cycle for a given target (e.g. for a spot in an image representing a given target) is not called or recorded as a signal code. In some embodiments, the dark cycles are not part of the coding scheme, or in other words a signal code in a signal code sequence is not an undetectable signal but comprises the presence of a detectable signal from a detectable label.

In some embodiments, a “sparse labelling” detection method is for detecting multiple target nucleic acid sequences in a sample, and thus the detection of the target nucleic acid sequences occurs in multiplex. That is to say that whilst there may be a dark cycle in respect of one target nucleic acid sequence, signals are being detected in respect of other target nucleic acid sequences, and corresponding signal codes are being identified. Accordingly, the individual signal codes which make up the signal code sequence for each of the target nucleic acid sequences may not be identified at the same time. In some embodiments, at a given point in the decoding process, e.g., after a given number of decoding cycles, it may be that more signal codes have been identified in respect of one target nucleic acid sequence than in respect of another.

In some embodiments, the “sparse labelling” detection method involves the use of at least one dark cycle, e.g., due to the omission of one or more hybridization probes for certain target nucleic acid sequences (e.g., barcode sequences in RCPs that are indicative of certain analytes in the sample) in the dark cycle. In some embodiments, the number of decoding cycles that are required to fully identify all of the individual signal codes which make up the signal code sequence for all of the target nucleic acid sequences is greater than the number of individual signal codes which make up the signal code sequences. In some embodiments, the number of decoding cycles required to fully identify all of the signal code sequences is greater than the number of hybridization probes in at least one set of hybridization probes, or greater than the number of signal code positions in a least one signal code sequence. Generally speaking the signal code sequences for different targets are designed to be of the same length, but this is not essential.

In some aspects, the present disclosure provides various formats of interfering agents capable of preventing signals associated with one or more selected probes from being generated and/or detected (e.g, for a dark cycle), or capable of reducing the level of detected signal associated with one or more selected. In some embodiments, the interfering agents (also referred to interchangeably as antidote probes) disclosed herein eliminate or reduce signal generation and/or detection associated with selected probes by manipulation of the analyte/probe binding (e.g., hybridization) and/or the probe/detection agent binding (e.g., hybridization). In some embodiments, interfering agents (antidote probes) disclosed herein eliminate or reduce signal generation and/or detection by quenching a detectable signal associated with a selected probe. In some embodiments, the interfering agents (antidote probes) disclosed herein eliminate or reduce signal generation and/or detection associated with selected probes by manipulation of hybridization, ligation, and/or amplification of selected circular or circularizable probe sets. The interfering agents disclosed herein may comprise any of a variety of entities that can hybridize to a nucleic acid (e.g., to a selected probe or a selected target nucleic acid sequence), typically by Watson-Crick base pairing, such as DNA, RNA, LNA, PNA, etc. In some embodiments, the interfering agents are interfering oligonucleotides (interfering oligos).

In some embodiments, the interfering agent (antidote probe) works by hybridizing to the selected hybridization probe (e.g., as shown in FIG. 4), or by hybridizing to the selected target hybridization sequence for the given cycle, resulting in a dark cycle for the selected target analyte (e.g., target nucleic acid molecule). These hybridization reactions may occur in any order. In some embodiments, the hybridization probes for the cycle and the antidote probe(s) for the cycle are added to the sample simultaneously. In some embodiments, the hybridization probes for the cycle are contacted with the at least one antidote probe for the cycle first, such that the hybridization probe-antidote probe complex can form, before the sample is contacted with the hybridization and antidote probes for the cycle. In some embodiments, the step of contacting the sample with the hybridization probes and the at least one antidote probe may comprise contacting the hybridization probes with the at least one antidote probe to provide a hybridization probe/antidote probe mixture for the cycle, and subsequently contacting the sample with the hybridization probe/antidote probe mixture for the cycle. In some embodiments, antidote probes can be provided in a plurality of sequential decoding rounds performed, wherein the antidote probes provided for a given decoding round correspond to the assigned “dark” cycles.

In some embodiments, there may be one or more steps of incubation to allow for the necessary hybridization reactions. In this regard, the hybridization probes may be incubated with the at least one antidote probe before they are added to the sample for the cycle. Additionally or alternatively, the mixture of hybridization probes and hybridization probe-antidote probe complexes may be incubated with the target nucleic acid sequences present in the sample before signal detection occurs.

Contacting/incubation steps may be followed by one or more washing steps, e.g. to remove probes that have not hybridized.

In some embodiments, each antidote probe comprises a sequence which is complementary to a sequence within the selected hybridization probe or the corresponding target nucleic acid. In some embodiments, the antidote probe is complementary to at least a portion of the recognition sequence of the hybridization probe, or complementary to at least a portion of the target nucleic acid sequence which the hybridization probe recognizes. As such, the antidote functions to block or otherwise attenuate binding of the hybridization probe to the target nucleic acid molecule, thereby reducing or eliminating detection of a signal from the target nucleic acid molecule and consequently reducing signal crowding.

In some embodiments, the hybridization probe is indirectly labelled and comprises a reporter domain for hybridizing a detection probe/reporter probe, and the antidote probe may be complementary to at least a portion of the reporter domain. As such, the antidote functions to block or otherwise attenuate binding of the reporter probe (e.g., comprising a detectable label) to the hybridization molecule, thereby reducing or eliminating detection of a signal from the target nucleic acid molecule in the dark cycle and consequently reducing signal crowding. In some embodiments, an antidote probe is complementary to the reporter domain of the hybridization probe, and the hybridization probe-antidote probe complex is able to bind to the target nucleic acid sequence, but the reporter probe will be unable to bind to the reporter domain of the hybridization probe, because it will be obstructed or blocked by the antidote probe. Thus, the “dark” cycle for a given target nucleic acid sequence can occur when the hybridization probe-antidote probe complex is unable to provide a signal which allows the target nucleic acid sequence to be identified.

In some embodiments, the interfering agents (antidote probes) are complementary to the reporter domain of the hybridization probe (and the hybridization probes are indirectly labelled using separate reporter probes), and the step of providing the one or more antidote probes occurs before the step of providing the set of reporter probes, such that the antidote probe can bind to the reporter domain of the hybridization probe before the cognate reporter probe. This will ensure that the hybridization probe-antidote probe complex can form correctly, so that a signal allowing the target nucleic acid sequence to be identified is not provided during the dark cycle.

In some embodiments, the interfering agent (antidote probe) is capable of hybridizing to the hybridization probe to form a hybridization probe-antidote probe complex which is not capable of providing a signal which allows the selected target nucleic acid sequence to be detected. If the antidote probe is complementary to the recognition sequence of the hybridization probe, the complex formed between the antidote probe and the hybridization probe (the hybridization probe-antidote probe complex) will be unable to bind to the target nucleic acid sequence, because the recognition sequence of the hybridization probe will be obstructed, or blocked, by the antidote probe. In other embodiments, a complex is formed between the antidote probe and the target nucleic acid, and the complex is unable to bind to the hybridization probe.

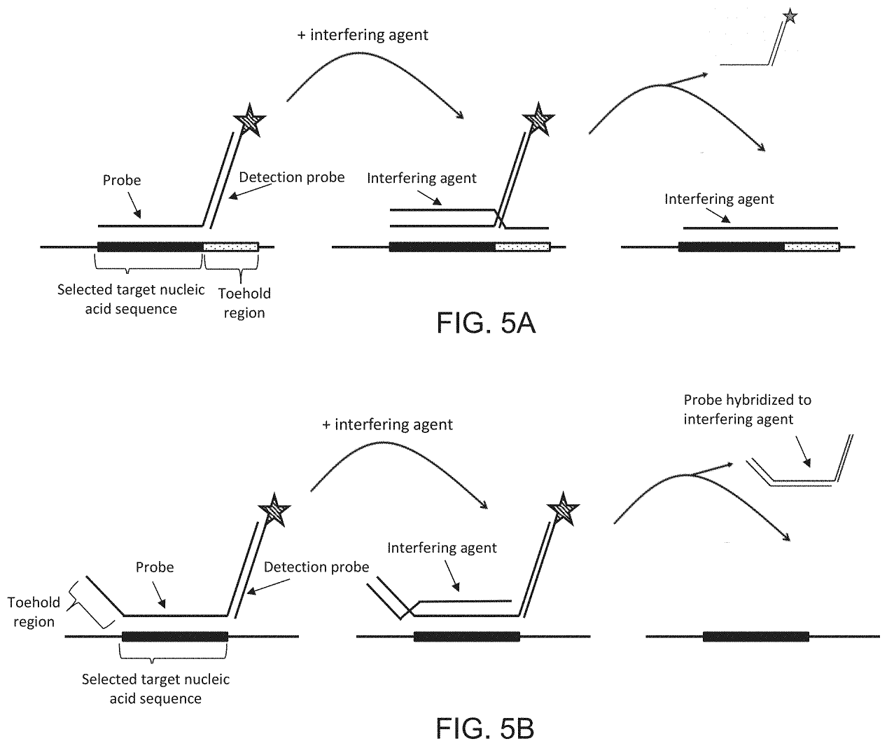

In some embodiments, the interfering agent (antidote probe) is capable of displacing the selected probe from its cognate target nucleic acid sequence, as shown in FIGS. 5A-5B. In some embodiments, the interfering agent comprises a sequence complementary to a toehold region that is a sequence adjacent to a selected target nucleic acid sequence, and a sequence complementary to the target nucleic acid sequence (as shown in FIG. 5A). In some embodiments, the toehold region is a single-stranded region in the target nucleic acid, and is available for hybridization by the interfering agent. In some embodiments, hybridization of the interfering agent to the toehold region can initiate a strand displacement reaction, wherein the interfering agent outcompetes the selected probe for hybridization to the selected target nucleic acid sequence, thereby displacing the selected probe. The displaced probe can be removed in a wash step. After displacement of the selected probe, the interfering agent can remain hybridized to the target nucleic acid sequence until it is optionally removed in a subsequent wash/stripping step. In some embodiments, the target nucleic acid sequence is in a nucleic acid analyte, a labelling agent, or a product of a nucleic acid analyte or labelling agent (e.g., a cDNA).

In some embodiments, the target nucleic acid sequence is in a probe that binds to the nucleic acid analyte or labelling agent. In some embodiments, the target nucleic acid sequence is in an overhang region of a probe that binds to the nucleic acid analyte or labelling agent, wherein the overhang region does not hybridize to the nucleic acid analyte or labelling agent. For example, in some instances the target nucleic acid sequence is an overhang region of an L-shaped probe (e.g., one that comprises a target recognition sequence and a 5′ or 3′ overhang upon hybridization to a target nucleic acid or a probe) or an overhang region of a U-shaped probe (e.g., one that comprises a target recognition sequence, a 5′ overhang, and a 3′ overhang upon hybridization to a target nucleic acid or a probe). In some embodiments, the target nucleic acid sequence is in a rolling circle amplification product. In some embodiments, the method comprises providing a circularizable probe (e.g., a padlock probe) that binds to a nucleic acid analyte or labelling agent in the sample such that the ends of the probe are juxtaposed for ligation; circularizing the probe, and generating a rolling circle amplification product from the circularized probe. In some instances, the circularized probe comprises the complement of the target nucleic acid sequence.

In some embodiments, the selected probe can comprise a toehold region adjacent to the recognition sequence that hybridizes to the target nucleic acid sequence. For example, a toehold region can be located on an overhang of the probe, as shown in FIG. 5B, and the interfering agent can hybridize to the toehold region of the selected probe. The interfering agent can thus comprise a sequence complementary to the toehold region of the selected probe, and a sequence complementary to the recognition sequence of the selected probe, such that hybridization of the interfering agent to the toehold region initiates a strand displacement reaction that releases a hybridization complex comprising the selected probe hybridized to the interfering agent from the selected target nucleic acid sequence. The released hybridization complex can be removed in a wash step.

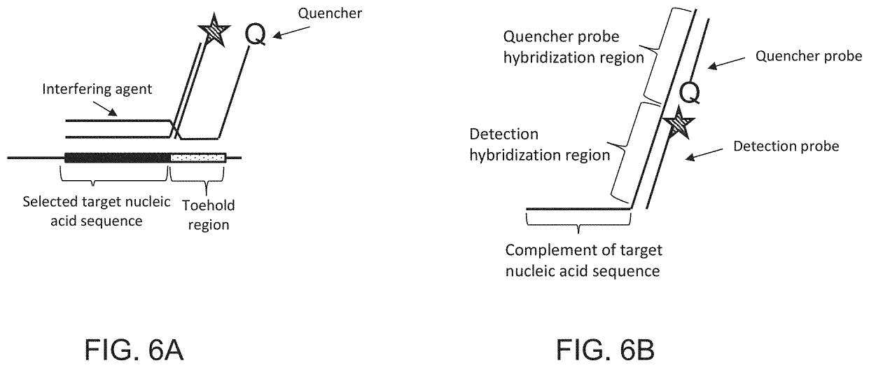

As discussed above, in some embodiments, an interfering agent can comprise a quencher moiety and can interfere with detection of a signal from selected probes (e.g., probes selected for a “dark” cycle) by quenching a detectable signal associated with said selected probes. In some embodiments, an interfering agent comprising a quencher moiety can also interfere with hybridization of a selected probe to its cognate target nucleic acid sequence, as shown in FIG. 6A. The inclusion of a quencher moiety can help further reduce or eliminate the signal associated with a selected probe. For example, in some embodiments, an interfering agent designed to displace a selected probe from a target nucleic acid may also comprise a quencher moiety, such that the quencher is brought into proximity with the detectable moiety (e.g., fluorescent moiety) of the selected probe during the displacement reaction, and can quench a signal of the detectable moiety if the interfering agent fails to completely displace the selected probe, as shown in FIG. 6A. Although FIG. 6A depicts an interfering agent comprising a quencher moiety hybridizing to a toehold region in the target nucleic acid (adjacent to the target nucleic acid sequence), it will be understood that the interfering agent comprising a quencher moiety could also be designed to hybridize to a toehold region within the probe, thereby initiating a strand displacement reaction (e.g., as shown in FIG. 5B).

In some aspects, an interfering agent can comprise a quencher moiety and can interfere with detection of a signal from selected probes for a dark cycle, without interfering with hybridization of said probes to their cognate target nucleic acids. In some embodiments, a hybridization probe of a probe mixture for detection of a plurality of analytes comprises a recognition sequence capable of hybridizing to a particular target nucleic acid sequence (e.g., the complement of the target nucleic acids sequence), a detection hybridization region (reporter region), and a quencher probe hybridization region, as shown in FIG. 5B. In some embodiments, the quencher probe hybridization region corresponds to the recognition sequence (e.g., the quencher probe hybridization region is specific for the target nucleic acid sequence). In this way, interfering agents comprising quencher moieties (quencher probes) can be designed to hybridize to the quencher probe hybridization region of selected hybridization probes (e.g., hybridization probes corresponding to highly abundant analytes). Thus, the selected hybridization probes can be selectively targeted for quenching during a dark cycle. As shown in FIG. 5B, in some embodiments, the quencher probe hybridizes to the selected hybridization probe such that the quencher moiety is brought into proximity with the detectable label (e.g., fluorescent moiety) of the detection/reporter probe that is hybridized to the same selected hybridization probe, thereby specifically quenching the signal associated with the selected hybridization probe.

Suitable quenchers are known in the art. In some embodiments, the quencher is a non-fluorescent quencher. Non-fluorescent quenchers have been described, for example, in WO200608406 and in U.S. Pat. No. 7,019,129, the contents of which are herein incorporated by reference in their entirety. Commonly used non-fluorescent quenchers include DABCYL, TAMRA, BlackHole Quenchers™ (BHQ, e.g. BHQ, BHQ1, or BHQ2), Biosearch Technologies, Inc. (Novato, Calif.), Iowa Black™, Integrated DNA Tech., Inc. (Coralville, Iowa), BlackBerry™ Quencher 650 (BBQ-650), Berry & Assoc., (Dexter, Mich.).

In some embodiments, each of the at least one antidote probes is provided at a concentration that is the same as or higher than the concentration at which the hybridization probe to which it will bind is provided. Generally, the antidote probes are used in excess of the hybridization probes. In some embodiments, the concentration of the antidote probe is at least 1.5 times greater, such as at least 2, 3, 4, 5 or 10 times greater than the concentration of the corresponding hybridization probe. In some embodiments, the concentration of the antidote probe is any one of at least 15, 20, 25, 30, 35, 40, 50, 60, 70, 80, 90, 100, 200, 300, or more times greater than the concentration of the corresponding hybridization probe. In some embodiments, by using an increased concentration of antidote probe, relative to hybridization probe, the formation of the hybridization probe-antidote probe complex is favored. This ensures that a signal from the selected target nucleic acid sequence(s) is not detected or is strongly reduced in the dark cycle.

In some embodiments, an antidote probe is provided at a final concentration (e.g., a final concentration in the probe mixture for contacting a sample) of between any of 0.5 uM and 1 uM, 0.75 uM and 1.5 uM, 1 uM and 2 uM, 1 uM and 5 uM, or 1 uM and 10 uM. In some embodiments, an antidote probe is provided at a final concentration of at least any one of 0.5 uM, 0.75 uM, 1 uM, 2 uM, 3 uM, 4 uM, or 5 uM. In some embodiments, an antidote probe is provided at a final concentration of no more than any one of 15 uM, 12 uM, 10 uM, 8 uM, 7.5 uM, 5 uM, 4 uM, 3 uM, 2 uM, or 1.5 uM.

In some embodiments, once the hybridization probes have hybridized to the target nucleic acid sequences, and the antidote probes have hybridized as necessary, the method comprises detecting a signal from each hybridization probe. This may involve detecting a signal from the detectable label of each hybridization probe which is directly or indirectly labelled with a detectable label. As noted above, signals are not detected from the hybridization probes for which antidote probes were provided, e.g. for the “dark” cycles. Instead, signals are only detected from the hybridization probes for which cognate antidote probes were not provided. The presence of the antidote probes prevents signals being generated from the selected target nucleic acid sequences for the dark cycle, and thus reduces signal crowding. This allows signals from the other, non-selected target nucleic acid sequences to be detected and resolved.

The signals may be detected by any suitable means for detecting the relevant detectable labels. In some embodiments, the signals may be detected by imaging the sample of target nucleic acid sequences. For example, if the detectable labels are fluorescent, the signals may be detected using fluorescence microscopy to determine the identity of the fluorescent label. It will be evident that other appropriate imaging techniques to identify signals from suitable detectable moieties may be used in the present methods to detect a signal from the label of a hybridization probe.

In some embodiments, the step of detecting signal from the labels of the hybridization probes which have hybridized to their respective target sequences may further comprise a step of removing unhybridized probes, prior to detecting the signals. The removal of the unhybridized probes may improve the strength of the signal that is detected, or the signal to noise ratio. This removal step may be carried out by washing the target nucleic acid sequences with an appropriate wash buffer. The step of washing the may be repeated multiple times, e.g. 2, 3, 4, 5 or more times, as is necessary.

In some embodiments, the signals that are detected from the hybridization probes which have hybridized to their target sequences allow for the target nucleic acid sequences which are not targeted by antidote probe(s) (e.g., where hybridization probes are contacted with antidote probes before contacting with the sample) to be identified based on the signals detected, thereby detecting the target nucleic acid sequences within the sample.

In some embodiments, each hybridization probe is capable of giving rise to signal by being detected, either directly or indirectly. As noted above, this may be the presence or absence of signal. Different hybridization probes may be detected, or distinguished from one another, by different labels, or by absence of a detectable label. In some embodiments, each hybridization probe may be directly or indirectly labelled with a detectable label which gives rise to a signal which may be recorded and/or assigned (e.g., serially) a signal code. In some embodiments, each hybridization probe is capable of hybridizing to a different target nucleic acid sequence (e.g., barcode sequence corresponding to a target analyte) and providing a signal. In some embodiments, a signal may include the signal detectable from the detectable label, and different detectable labels may provide different signals which may be distinguished, e.g. by color. In some embodiments, absence of signal may also be recorded and/or assigned a signal code. In some embodiments, in a plurality of hybridization probes, one or more of the probes may be lacking a detectable label, and thus the absence of a signal may be recorded and analyzed, for example, by assigning a signal code to the absence of signal (also known as a “dark” cycle for the one or more of the probes and the corresponding analyte(s)). In some embodiments, when there is a single cycle of detection to detect the signals from the hybridization probes, the plurality of hybridization probes may comprise molecules of one hybridization probe which is not labelled, and the remainder of the probes may comprise detectable labels which can be distinguished from one another. In some embodiments, a combinatorial, e.g. sequential, labelling scheme is used (e.g., multiple cycles of sequential signal detection), and the plurality of hybridization probes for different analytes (or different barcode sequences corresponding to the same or different analytes) used in a given cycle need not all be distinguishable from one another in terms of the signal (e.g., may comprise the same detectable label, such as the same color of fluorophore), as it is the combination (e.g., sequence or order) of signals which identifies the target nucleic acid sequence, not a single signal.

The detectable label may be any detectable moiety and may be directly or indirectly linked to the hybridization probe. The hybridization probe may thus be considered to be directly or indirectly signal-giving. In some embodiments, the detectable label is incorporated into the hybridization probe. For example, the detectable label may be linked directly (e.g., covalently) or via a linker (e.g., a chemical or nucleic acid linker) to the target nucleic acid recognition sequence of the hybridization probe.

In some embodiments, the hybridization probe (e.g., bound to the target nucleic acid) may provide a signal indirectly, e.g., via one or more further components (e.g., detectably labeled probes that bind the hybridization probe) to generate a signal. For instance, the hybridization probe may comprise a domain which is capable of binding a species that comprises a detectable label. In some embodiments, the hybridization probe comprises a detection hybridization region (also referred to as a reporter domain) which is not complementary to, and does not bind to, the target nucleic acid sequence but which comprises a binding site for a detection probe (also referred to as a reporter probe) which comprises a detectable label. More particularly, the detection hybridization region/reporter domain of the hybridization probe may comprise a binding site in the form of a nucleotide sequence comprising a region or domain to which a complementary detection/reporter probe may hybridize. In some embodiments, the nucleotide sequence of the detection/reporter domain is not complementary to and does not hybridize to the target nucleic acid sequence.

In some embodiments, the detection/reporter domain may be in the form of an overhang region of the hybridization probe, which is not complementary to the target nucleic acid sequence, but which comprises a binding site that is complementary to the sequence of a detection/reporter probe. In some embodiments, the detection/reporter probe comprises a cognate sequence which is complementary to that of the binding site in the reporter domain, and a detectable label.

In some embodiments, a method disclosed herein comprises providing a plurality of hybridization probes each specific for a target nucleic acid and a set of detection probes/reporter probes, cognate for the hybridization probes. The detection/reporter probes may be used separately from the hybridization probes and that they do not necessarily need to be provided together or at the same time. For instance, the detection/reporter probes (as well as the interfering agents such as antidote probes) may be contacted with the sample at a separate time, or in separate step, to contacting with the hybridization probes. For example, the sample may be contacted with the detection/reporter probes after contacting with the hybridization probes and the antidote probes, for example during the detecting step. In some embodiments, a detection/reporter probe and a hybridization probe are cognate to each other in that the detection/reporter probe corresponds to and is designed to bind to the hybridization probe (e.g., via a reporter domain on the hybridization probe) .

In some embodiments, a detection/reporter probe (e.g., a fluorescently labelled detection oligo) herein comprises a sequence which is complementary to that of a reporter domain (the detection hybridization region or reporter probe binding site) in a hybridization probe. In some embodiments, each detection/reporter probe comprises a detectable label. In some embodiments, a plurality of different sets of detection/reporter probes are provided, each set with a type of detectable label. The detectable label for each set of detection/reporter probes may be different, for example, the detectable label for each set can be a different fluorophore detectable in a separate fluorescence channel of a microscope. The plurality of hybridization probes and the sets of reporter probes may be provided simultaneously or sequentially. In an embodiment, a mixture of hybridization probes and detection/reporter probes may be prepared and added to or contacted with the sample. Further, in some embodiments, the reporter probes may be pre-hybridized to the hybridization probes. In other embodiments, the detection/reporter probes are hybridized to the hybridization probes after they have hybridized to their target sequence(s), or after the hybridization probes have been allowed to hybridize to their target sequence(s), and the antidote probes have been allowed to hybridize to the hybridization probes or the target sequence(s).

Detectable labels that may be used according to the methods herein, either in hybridization probes, or in detection/reporter probes, include any moiety capable of providing a signal that can be detected, for example fluorescent molecules (e.g. fluorescent proteins or organic fluorophores), colorimetric moieties (e.g. colored molecules or nanoparticles), particles, for example gold or silver particles, quantum dots, radioisotopes, chemiluminescent molecules, and the like. Any detectable moiety may be used as the detectable label. In particular, any spectrophotometrically or optically detectable label may be used. In some embodiments, the detectable label may be optically detectable. The detectable label may be distinguishable by color, but any other parameter may be used e.g. size or intensity.

In an embodiment, the hybridization probe or the reporter probe comprises a fluorescent label. This may be a fluorescent molecule, e.g. a fluorophore. Exemplary fluorophores include ATTO dyes (such as ATTO 425, ATTO 550, ATTO 647(N), ATTO 655), cyanine dyes (e.g., Cy3, Cy5, Cy7), and Alexa Fluor dyes (such as AF 488, AF555, AF 647, AF 750), though any suitable fluorophores may be used. Fluorophores have been identified with excitation and emission spectra ranging from UV to near IR wavelengths. Thus, the fluorophore may have an excitation and/or emission wavelength in the UV, visible or IR spectral range. In some instances, the fluorophore is a green fluorescent protein, a blue fluorescent protein, a yellow fluorescent protein, an orange fluorescent protein, a red fluorescent protein, a far-red fluorescent protein, or a near-IR fluorescent protein, or any combinations thereof. The fluorophore may be a peptide, small organic compound, synthetic oligomer or synthetic polymer. In some embodiments, the fluorophore is a small organic compound.

In some embodiments, a reporter probe (also referred to as detection probe, e.g., detection oligo) may comprise no detectable label. In this case, the signal that is reported is the absence of any detectable label, which is distinguishable from any number of distinct positively detectable labels.

### V. Compositions and Kits

In some aspects, disclosed herein is a composition that comprises a complex containing a target nucleic acid and one or more probes or probe sets (e.g., primary, secondary probes and/or detection probes any as described herein). In some embodiments, the composition further includes a primer for amplification of the probe or probe sets. In some embodiments, the composition comprises a target nucleic acid or multiple target nucleic acids and an amplification product of the probe.

Also provided herein are kits comprising one or more probes, including any as described in Sections II-IV, and reagents for performing the methods provided herein, for example reagents required for one or more steps comprising hybridization, ligation, amplification, detection, sequencing, and/or sample preparation as described herein. In some embodiments, the kit further comprises a target nucleic acid. In some embodiments, any or all of the polynucleotides are DNA molecules. In some embodiments, the target nucleic acid is a messenger RNA molecule. In some embodiments, the kit further comprises a ligase. In some embodiments, the kit further comprises a polymerase, for instance for performing amplification of a circular or circularizable probe set (e.g., a padlock probe). In some embodiments, the kit further comprises a primer for amplification. In some embodiments, the kit further comprises probes for a subset of the target nucleic acid sequences that are not labelled.

Also provided herein is a kit for use in the detection method described above. In some embodiments, the kit further comprises one or more interfering agents (e.g., antidote probe), wherein each interfering agent interferes with hybridization of the selected probe to its corresponding nucleic acid sequence in or associated with the corresponding analyte. In some embodiments, the kit comprises: (a) a plurality of hybridization probes comprising different hybridization probes each specific for a different target nucleic acid sequence, wherein each hybridization probe has a recognition sequence complementary to a sequence within its target nucleic acid sequence, and is directly or indirectly labelled with a detectable label which gives rise to a signal; and (b) at least one antidote probe for at least one selected hybridization probe specific for a selected target nucleic acid sequence. In some embodiments, each antidote probe comprises a sequence complementary to a sequence within the selected hybridization probe and is capable of hybridizing to it to form a hybridization probe-antidote probe complex which is not capable of providing a signal which allows the selected target nucleic acid sequence to be detected. In some embodiments, each antidote probe comprises a sequence complementary to a sequence within the selected target nucleic acid sequence and is capable of hybridizing to it to form a selected target nucleic acid sequence-antidote probe complex which is not capable of providing a signal which allows the selected target nucleic acid sequence to be detected, for example, because the antidote probe prevents or reduces hybridization of the selected hybridization probe to the selected target nucleic acid sequence.

The disclosures above relating to the structures of the hybridization probes and the antidote probes apply equally in relation to the hybridization probes and antidote probes which make up the kit.

In some embodiments, the number of antidote probes is less than the number of hybridization probes, e.g., in cases where one antidote probe is provided for each target nucleic acid sequence for which a signal is to be suppressed or silenced. In some embodiments, the kit is arranged such that not every hybridization probe has a corresponding antidote probe. In some embodiments, the number of antidote probes in the kit may be less than 50%, such as less than 40%, 30%, 25%, 20%, 15%, 10% or 5% of the number of hybridization probes in the kit.

In some embodiments, the kit comprises n sets of probes:
Probe Set 1 comprises P11, . . . , P1j, . . . , and P1m, for target nucleic acid sequence T1,
Probe Set k comprises Pk1, . . . , Pkj, . . . , and Pkm, for target nucleic acid sequence Tk,
Probe Set n comprises Pn1, . . . , Pnj, . . . , and Pnm, for target nucleic acid sequence Tn,

wherein j, k, m, and n are integers, 2≤j≤m, and 2≤k≤n, and the n sets of probes are used to decode signal code sequences for target nucleic acid sequences T1, . . . , Tk, . . . , Tn, in m cycles. In some embodiments, each probe is detectable by a fluorescently labelled reporter probe, and the fluorescent signals for different probes in each probe set or each probe library can be of the same or different colors. In some embodiments, n (the number of target nucleic acid sequences to be detected) is at least 5, 10, 20, 50, 100, 200, 500, 1,000, 2,000, 5,000, or 10,000, or greater than 10,000. In some embodiments, m (the number of cycles) is at least 2, 3, 4, 5, 6, 7, 8, 9, 10, 11, 12, 13, 14, 15, 16, 17, 18, 19, 20, or greater than 20. In some embodiments, the sample is contacted with Probe Library P11, . . . , Pk1, . . . , and Pn1 in Cycle 1, with Probe Library P1j, Pkj, . . . , and Pnj in Cycle j, and Probe Library P1m, Pkm, . . . , and Pnm in Cycle m.

In some embodiments, in one or more of Cycle 1 through Cycle m, the sample is contacted with an interfering oligo that hybridizes to a target nucleic acid sequence or the corresponding probe(s), and the interfering oligo is not detectably (e.g., fluorescently) labelled. In some embodiments, the kit further comprises interfering oligos that hybridizes to all of the probes in any one or more of Probe Set 1 to Probe Set n. For example, when target nucleic acid sequence Tk is an abundant or highly expressed in the sample, an interfering oligo Ik that hybridizes to Tk or the corresponding probes Pk1, . . . , Pkj, . . . , and Pkm may be included in the kit. In some examples, interfering oligo Ik hybridizes to all of probes Pk1, . . . , Pkj, . . . , and Pkm and prevents these probes from hybridizing to Tk and providing detectable signals indicative of Tk in the sample. For instance, the sample can be contacted with Probe Library P11, . . . , Pk1, . . . , and Pn1 and Ik (antidote for Pk1) in Cycle 1, with Probe Library P1j, . . . , Pkj, . . . , and Pnj and Ik (antidote for Pkj) in Cycle j, and with Probe Library P1m, . . . , Pkm, . . . , and Pnm and Ik (antidote for Pkm) in Cycle m. In some embodiments, the kit comprises hybridization probes and antidote probes for each cycle in a pre-mix which is then contacted with the sample. In some embodiments, a kit disclosed herein comprises any one or more of m compositions: Composition No. 1 comprising P11, . . . , Pk1, . . . , and Pn1 and Ik (antidote for Pk1), . . . , Composition No. j comprising P1j, . . . , Pkj, . . . , and Pnj and Ik (antidote for Pkj), . . . , and Composition No. m comprising P1m, . . . , Pkm, . . . , and Pnm and Ik (antidote for Pkm).

The various components of the kit may be present in separate containers or certain compatible components may be pre-combined into a single container. In some embodiments, the kits further contain instructions for using the components of the kit to practice the provided methods. In some embodiments, probes (e.g., with detectable labels) can be provided in a manner such that probes for one or more target nucleic acid sequences can be omitted.

In some embodiments, the kits can contain reagents and/or consumables required for performing one or more steps of the provided methods. In some embodiments, the kits contain reagents for fixing, embedding, and/or permeabilizing the biological sample. In some embodiments, the kits contain reagents, such as enzymes and buffers for ligation and/or amplification, such as ligases and/or polymerases. In some aspects, the kit can also comprise any of the reagents described herein, e.g., wash buffer and ligation buffer. In some embodiments, the kits contain reagents for detection and/or sequencing, such as barcode detection probes or detectable labels. In some embodiments, the kits optionally contain other components, for example nucleic acid primers, enzymes and reagents, buffers, nucleotides, modified nucleotides, reagents for additional assays.

### VI. Applications

The methods disclosed herein are methods for detecting multiple target nucleic acid sequences in a sample. These methods may be used in a variety of different applications, and thus the identity of the sample and of the target nucleic acid sequences may be varied. Any means of determining the presence of the target nucleic acid sequences (e.g., if they are present or not) or any form of measurement of the target nucleic acid sequences may be employed. A method disclosed herein may comprise determining, measuring, assessing and/or assaying the presence or absence or amount or location of the target nucleic acid sequences in any way.

In some embodiments, a method disclosed herein may comprise using a sequential decoding scheme for localized detection of target nucleic acid sequences in a sample. In some embodiments, in a localized detection, the signals giving rise to the detection of the target nucleic acid sequences are localized to those sequences. In turn, the target nucleic acid sequences are localized in the sample, that is they are present at, and remain at, a given or specific location in the sample. The target nucleic acid sequences may therefore be detected in or at their locations in the sample. In some embodiments, the spatial position (or localization) of the target nucleic acid sequences within the sample may be determined (or “detected”). This means, for example, that the target nucleic acid sequences may be localized to, or within, a cell or tissue in which they are expressed, or to a position within a cell or tissue sample in which they are present. A target nucleic acid sequence which is not itself the target analyte of an assay, but which is generated therefrom, or used or generated as a reporter therefor may be localized to the target analyte, and hence in the sample, by virtue of being bound to or otherwise associated with the analyte. Thus “localized detection” may include determining, measuring, assessing or assaying the presence or amount and location, or absence, of the target nucleic acid sequences in any way.

More particularly, the methods may be used for the in situ detection of target nucleic acid sequences, or of a target analyte for which the target nucleic acid sequence is a reporter. In a particular embodiment, the methods may be used for the localized, particularly in situ, detection of mRNA sequences. More particularly, the methods may be used for the localized, particularly in situ, detection of mRNA sequences in a sample of cells.

In some embodiments, an in situ assay comprises the detection of target nucleic acid sequences, or target analytes, in their native contexts, e.g. in the cell or tissue in which they normally occur. Thus, this may refer to the natural or native localization of target nucleic acid sequences or target analytes. In other words, the target nucleic acid sequences may be detected where, or as, they or the target analytes in respect of which they are to be detected, occur in their native environment or situation. Thus, the target nucleic acid sequences or analytes are not moved from their normal location, e.g. are not isolated or purified in any way, or transferred to another location or medium etc. In some embodiments, an in situ assay comprises the detection of the target nucleic acid sequences or analytes as they occur within a cell or within a cell or tissue sample, e.g. their native localization within the cell or tissue and/or within their normal or native cellular environment. In particular, in situ detection includes detecting the target nucleic acid sequences within a tissue sample, and particularly a tissue section. In other embodiments the methods can be carried out on a sample of isolated cells, such that the cells themselves are not in situ.

In some embodiments, a sparse labelling detection method comprises multiple sequential decoding cycles, wherein the signal code sequence for each target nucleic acid sequence is determined by detecting signals from individual hybridization probes across multiple cycles. It will therefore be understood, that in order for the signal code sequences for each target nucleic acid sequence to be built up, the target nucleic acid sequences must be fixed in position, or immobilized. If the target nucleic acid sequences were not each located at a single site or position (e.g., immobilized) in the sample, it would not be possible to identify a sequential set of signals which were detected from the same target nucleic acid sequence, and thus the signal code sequences could not be correctly determined. In some embodiments, this immobilization may occur by virtue of the target nucleic acid sequences being present in situ in a sample, or being bound to or associated with a target analyte which is present in situ. In other embodiments, this may be done by immobilizing the target nucleic acid sequences in situ, for example, the target nucleic acid sequences may be immobilized, or fixed, as they occur in the sample, e.g. in cells. For example, a tissue sample may be fixed or immobilized, or cells may be taken from a sample, which may be tissue or body fluid sample, or indeed a culture or any sample containing cells, and the cells may be fixed or immobilized onto a solid surface. In such a situation, whilst the cells may no longer be in a native in situ context, the target analyte/nucleic acid sequence may remain in an in situ context within the cell. In still other embodiments, the target nucleic acid sequences, or their corresponding target analytes (e.g. the analytes to which they bind or become bound or associated etc.) may be removed from their native in situ context and immobilized on a solid surface. In this way, the target nucleic acid sequence or target analyte may be localized at a particular identifiable site or location, and may remain there during the performance of the method such that, in particular, the location does not change, and remains the same, from cycle to cycle.

Accordingly, the “sparse labelling” method is not necessarily limited to localized detection in situ; the target nucleic acid sequences may alternatively be localized by being immobilized on a solid support not in the context of their original or native location. In this context, the target nucleic acid sequences are isolated from their original environment, and thus it will be understood that information about the location of the target nucleic acid sequences within that environment will not be available.

Further, this method include embodiments which are not in situ, in the sense that the target nucleic acid sequences, or their corresponding or respective target analytes, are not present (e.g. not fixed) in their native contexts. This may include embodiments in which target nucleic acid sequences are immobilized, directly or indirectly, e.g. on a solid support.

The target nucleic acid sequences are present within a sample. The sample may be any sample which contains any amount of target nucleic acid sequences which are to be detected, from any source or of any origin. A sample may thus be any clinical or non-clinical sample, and may be any biological, clinical or environmental sample in which the target nucleic acid sequences may occur. All biological and clinical samples are included, e.g. any cell or tissue sample of an organism, or any body fluid or preparation derived therefrom, as well as samples such as cell cultures, cell preparations, cell lysates etc. Environmental samples, e.g. soil and water samples or food samples are also included. The samples may be freshly prepared for use in the methods of the present disclosure, or they may be prior-treated in any convenient way e.g. for storage.

As noted above, in one embodiment, the target nucleic acid sequences may be detected in situ, as they naturally occur in the sample. In such an embodiment the target nucleic acid sequences may be present in a sample at a fixed, detectable or visualisable position in the sample. The sample will thus be any sample which reflects the normal or native (“in situ”) localization of the target nucleic acid sequences, e.g. any sample in which they normally or natively occur. Such a sample will advantageously be a cell or tissue sample. Particularly preferred are samples such as cultured or harvested or biopsied cell or tissue samples in which the target nucleic acid sequences may be detected to reveal the localization of the target nucleic acid sequences relative to other features of the sample. Thus, the in situ context may be the context of a cell. In another embodiment the in situ context may be the in the context of a tissue which contains the cell, etc. Accordingly, in some embodiments, the sample may be a cell or tissue sample, in particular a human tissue sample. In some embodiments, the sample may be a cancer tissue sample.

The target nucleic acid sequences within the sample may be any target nucleic acid sequences which it is desired to detect. In some embodiments, the target nucleic acid sequence may be target analyte nucleic acid sequences. The target analyte nucleic acid sequences may be any nucleic acid sequences, including DNA, RNA, or a mixture thereof. Moreover, the target analyte nucleic acid sequences may be any form of nucleic acid, such as mRNA, cDNA, etc. As noted above, in a particularly preferred embodiment, the target nucleic acid sequences are mRNA sequences.

The target nucleic acid sequences may be nucleic acid sequences generated from target analyte nucleic acid sequences, such as amplicons or complementary copies of target analyte nucleic acid sequences. In some embodiments, the RNA sequences present in the sample may be reverse transcribed into cDNA sequences, for example by contacting the sample with a reverse transcriptase enzyme and appropriate primers. In such an embodiment, the cDNA sequences produced by the reverse transcription reaction can then be considered as the target nucleic acid sequences to be detected.

Furthermore, the target nucleic acid sequences may be nucleic acid sequences which are generated as reporters for other target analytes. In such an embodiment, target nucleic acid sequences may be provided to, e.g. added to or generated in a sample (e.g. they may be nucleic acid sequences that were not present in the original sample). A target nucleic acid sequence may be provided in the sample as a tag or reporter for a target analyte, for example by one or more molecules that interact with, e.g. bind to, target analytes. The detection of the added or generated target nucleic acid sequences is thus indicative of the presence of the alternative target analytes in a sample.

In such a context the target analyte may be any target molecule, including nucleic acid molecules, or analytes other than nucleic acid molecules, such as a proteins, peptides, carbohydrates etc. Various methods based upon detecting such a reporter target nucleic acid sequence, which is indicative of an underlying target analyte, are well described in the art, including, for example, immunoRCA and immunoPCR as noted above, and assays using padlock probes or proximity probes. The use of proximity probes comprising analyte-binding domains and nucleic acid domains which interact upon binding of the probes to a target analyte is widely described in the literature. In the context of proximity probes, a target nucleic acid sequence may be generated by extension or ligation of nucleic acid domains of proximity probes, or of additional nucleic acid molecules (e.g. oligonucleotides) which hybridize to the nucleic acid domain of a proximity probe. In the context of a padlock probe, a target nucleic acid sequence may be generated as the RCP of that probe, or indeed simply a result of ligation of the probe. In the context of immunoPCR or immunoRCA, a PCR or RCA product may be generated to provide a target nucleic acid sequence. Still further, a target nucleic acid sequence may be added to the sample as the nucleic acid domain of an analyte-binding probe.

For example, a protein or other analyte in a sample may be detected by an antibody or other analyte-specific binding partner, which is provided with an oligonucleotide, and that oligonucleotide may be considered to be a target nucleic acid sequence. In this case, the hybridization probes may be designed to hybridize to that oligonucleotide, such that the oligonucleotide can be detected, and therefore can indicate the presence of the antibody, and hence the analyte. Similarly, an oligonucleotide sequence may be generated as part of an analyte detection assay, e.g. an extension or ligation product may be generated as the result of a proximity assay, and this oligonucleotide may be considered to be a target nucleic acid sequence.

In some embodiments, the “sparse labelling” detection method uses a circularizable probe specific for each target nucleic acid sequence which is circularized upon hybridization to the target nucleic acid sequence and is amplified by rolling circle amplification (RCA) to produce a rolling circle product (RCP). As noted above, the target nucleic acid sequences to which the circularizable probes are hybridized may be any nucleic acid sequences which are desired to be detected. The RCA reaction is used to amplify the signal which is generated from the target nucleic acid sequence, in order to improve the signal to noise ratio and therefore increase the utility of the detection method.

The circularizable probes from which the RCPs are generated may be as defined above. It will be clear that the circularizable probes will be circularized before the RCA reactions occur. In particular, the circularizable probes may be padlock probes. As outlined above, the padlock probes may each comprise a barcode sequence, wherein each padlock probe comprises a different barcode sequence specific for a different target nucleic acid sequence.

In some embodiments, the padlock probes may be specific for target nucleic acid analytes present in the sample. In a particular embodiment, the method may be used for the detection of mRNA sequences, and thus the padlock probes may be specific for mRNA sequences present in the sample. More particularly, the sample may be a sample of cells, and the mRNA sequences may be detected in situ. Accordingly, the padlock probes may hybridize to and circularize on the mRNA sequences present in the cells in the sample. When the padlock probes are amplified by RCA, the resulting RCPs will each comprise multiple complementary copies of the barcode sequence from the relevant padlock probe. The barcode sequences will allow the RCPs (the target nucleic acid sequences) to be detected using the decoding methods outlined above, and therefore will in turn allow the mRNA sequences to be indirectly detected.

The present detection methods reduce signal crowding by reducing the number of signals which are generated and detected at any one time. As set out above, this may be done by omitting hybridization probes (or the detectable labels thereof) from decoding cycles and it is possible to vary the extent to which these strategies are employed (e.g. to vary the number of dark cycles which are used), depending on the degree of signal crowding which is experienced.

In some embodiments, any one or more target nucleic acid sequences may be selected and hybridization probes are omitted. In some embodiments, in order to reduce the signal crowding to the largest possible extent, it may be desirable to target the signal crowding reduction strategies against the specific target nucleic acid sequences which are significantly responsible for causing the signal crowding problem. Accordingly, in some embodiments, the target nucleic acid sequences which are selected (for which hybridization probes are to be omitted) are target nucleic acid sequences which are present in the sample in an increased amount relative to other target nucleic acid sequences in the sample.

For example, if the detection methods are to be used to detect multiple target mRNA sequences in order to assess gene expression in a particular cell or tissue sample, the target nucleic acid sequences which are selected (for which hybridization probes are to be omitted) may be target mRNA sequences corresponding to a gene or genes which is expressed in an increased amount relative to other genes in the sample.

The selection of target nucleic acid sequences which are present in the sample in an increased amount relative to other target nucleic acid sequences may be informed by prior knowledge of the sample in question. In the example provided above of a gene expression analysis, the skilled person may be aware of genes which are likely to be highly expressed within the sample in question, and may select target nucleic acid sequences accordingly. That is to say that the skilled person may be able to use the common general knowledge in the field to select appropriate target nucleic acid sequences.

Alternatively, the selection may be based on the results of previous experiments. In this regard, in some embodiments, the detection methods may comprise preceding steps of identifying target nucleic acid sequences which cause signal crowding.

In the context of the “sparse labelling” detection method, the method may comprise preceding steps of detecting target nucleic acid sequences in the sample using the sets of hybridization probes and determining the presence in the sample of target nucleic acid sequences which give rise to signals which crowd out signals from other target nucleic acid sequences in the sample, wherein those target sequences are selected for one or more sparse cycles for detection.

### VII. Terminology

Specific terminology is used throughout this disclosure to explain various aspects of the apparatus, systems, methods, and compositions that are described.

Having described some illustrative embodiments of the disclosure, it should be apparent to those skilled in the art that the foregoing is merely illustrative and not limiting, having been presented by way of example only. Numerous modifications and other illustrative embodiments are within the scope of one of ordinary skill in the art and are contemplated as falling within the scope of the present disclosure. In particular, although many of the examples presented herein involve specific combinations of method acts or system elements, it should be understood that those acts and those elements may be combined in other ways to accomplish the same objectives.

As used herein, the singular forms “a,” “an,” and “the” include plural referents unless the context clearly dictates otherwise. For example, “a” or “an” means “at least one” or “one or more.”

The term “about” as used herein refers to the usual error range for the respective value readily known to the skilled person in this technical field. Reference to “about” a value or parameter herein includes (and describes) embodiments that are directed to that value or parameter per se.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

### (i) Barcode

A “barcode” is a label, or identifier, that conveys or is capable of conveying information (e.g., information about an analyte in a sample). A barcode can be part of an analyte, or independent of an analyte. A barcode can be attached to an analyte. A particular barcode can be unique relative to other barcodes.

Barcodes can have a variety of different formats. For example, barcodes can include polynucleotide barcodes, random nucleic acid and/or amino acid sequences, and synthetic nucleic acid and/or amino acid sequences. A barcode can be attached to an analyte or to another moiety or structure in a reversible or irreversible manner. A barcode can be added to, for example, a fragment of a deoxyribonucleic acid (DNA) or ribonucleic acid (RNA) sample before or during sequencing of the sample. Barcodes can allow for identification and/or quantification of individual sequencing-reads (e.g., a barcode can be or can include a unique molecular identifier or “UMI”).

Barcodes can spatially-resolve molecular components found in biological samples, for example, at single-cell resolution (e.g., a barcode can be or can include a “spatial barcode”). In some embodiments, a barcode includes both a UMI and a spatial barcode. In some embodiments, a barcode includes two or more sub-barcodes that together function as a single barcode. For example, a polynucleotide barcode can include two or more polynucleotide sequences (e.g., sub-barcodes) that are separated by one or more non-barcode sequences.

### (ii) Nucleic Acid and Nucleotide

The terms “nucleic acid” and “nucleotide” are intended to be consistent with their use in the art and to include naturally-occurring species or functional analogs thereof. Particularly useful functional analogs of nucleic acids are capable of hybridizing to a nucleic acid in a sequence-specific fashion (e.g., capable of hybridizing to two nucleic acids such that ligation can occur between the two hybridized nucleic acids) or are capable of being used as a template for replication of a particular nucleotide sequence. Naturally-occurring nucleic acids generally have a backbone containing phosphodiester bonds. An analog structure can have an alternate backbone linkage including any of a variety of those known in the art. Naturally-occurring nucleic acids generally have a deoxyribose sugar (e.g., found in deoxyribonucleic acid (DNA)) or a ribose sugar (e.g. found in ribonucleic acid (RNA)).

A nucleic acid can contain nucleotides having any of a variety of analogs of these sugar moieties that are known in the art. A nucleic acid can include native or non-native nucleotides. In this regard, a native deoxyribonucleic acid can have one or more bases selected from the group consisting of adenine (A), thymine (T), cytosine (C), or guanine (G), and a ribonucleic acid can have one or more bases selected from the group consisting of uracil (U), adenine (A), cytosine (C), or guanine (G). Useful non-native bases that can be included in a nucleic acid or nucleotide are known in the art.

(iii) Probe and Target

A “probe” or a “target,” when used in reference to a nucleic acid or sequence of a nucleic acids, is intended as a semantic identifier for the nucleic acid or sequence in the context of a method or composition, and does not limit the structure or function of the nucleic acid or sequence beyond what is expressly indicated.

### (iv) Oligonucleotide and Polynucleotide

The terms “oligonucleotide” and “polynucleotide” are used interchangeably to refer to a single-stranded multimer of nucleotides from about 2 to about 500 nucleotides in length. Oligonucleotides can be synthetic, made enzymatically (e.g., via polymerization), or using a “split-pool” method. Oligonucleotides can include ribonucleotide monomers (e.g., can be oligoribonucleotides) and/or deoxyribonucleotide monomers (e.g., oligodeoxyribonucleotides). In some examples, oligonucleotides can include a combination of both deoxyribonucleotide monomers and ribonucleotide monomers in the oligonucleotide (e.g., random or ordered combination of deoxyribonucleotide monomers and ribonucleotide monomers). An oligonucleotide can be 4 to 10, 10 to 20, 21 to 30, 31 to 40, 41 to 50, 51 to 60, 61 to 70, 71 to 80, 80 to 100, 100 to 150, 150 to 200, 200 to 250, 250 to 300, 300 to 350, 350 to 400, or 400-500 nucleotides in length, for example. Oligonucleotides can include one or more functional moieties that are attached (e.g., covalently or non-covalently) to the multimer structure. For example, an oligonucleotide can include one or more detectable labels (e.g., a radioisotope or fluorophore).

### (v) Adaptor, Adapter, and Tag

An “adaptor,” an “adapter,” and a “tag” are terms that are used interchangeably in this disclosure, and refer to species that can be coupled to a polynucleotide sequence (in a process referred to as “tagging”) using any one of many different techniques including (but not limited to) ligation, hybridization, and tagmentation. Adaptors can also be nucleic acid sequences that add a function, e.g., spacer sequences, primer sequences/sites, barcode sequences, unique molecular identifier sequences.

### (vi) Hybridizing, Hybridize, Annealing, and Anneal

The terms “hybridizing,” “hybridize,” “annealing,” and “anneal” are used interchangeably in this disclosure, and refer to the pairing of substantially complementary or complementary nucleic acid sequences within two different molecules. Pairing can be achieved by any process in which a nucleic acid sequence joins with a substantially or fully complementary sequence through base pairing to form a hybridization complex. For purposes of hybridization, two nucleic acid sequences are “substantially complementary” if at least 60% (e.g., at least 70%, at least 80%, or at least 90%) of their individual bases are complementary to one another.

(vii) Primer

A “primer” is a single-stranded nucleic acid sequence having a 3′ end that can be used as a substrate for a nucleic acid polymerase in a nucleic acid extension reaction. RNA primers are formed of RNA nucleotides, and are used in RNA synthesis, while DNA primers are formed of DNA nucleotides and used in DNA synthesis. Primers can also include both RNA nucleotides and DNA nucleotides (e.g., in a random or designed pattern). Primers can also include other natural or synthetic nucleotides described herein that can have additional functionality. In some examples, DNA primers can be used to prime RNA synthesis and vice versa (e.g., RNA primers can be used to prime DNA synthesis). Primers can vary in length. For example, primers can be about 6 bases to about 120 bases. For example, primers can include up to about 25 bases. A primer may, in some cases, refer to a primer binding sequence.

(viii) Primer Extension

A “primer extension” refers to any method where two nucleic acid sequences become linked (e.g., hybridized) by an overlap of complementary nucleic acid sequences (e.g., for example, 3′ termini). Such linking can be followed by nucleic acid extension (e.g., an enzymatic extension) of one, or both termini using the other nucleic acid sequence as a template for extension. Enzymatic extension can be performed by an enzyme including, but not limited to, a polymerase and/or a reverse transcriptase.

### (ix) Proximity Ligation

A “proximity ligation” is a method of ligating two (or more) nucleic acid sequences that are in proximity with each other through enzymatic means (e.g., a ligase). In some embodiments, proximity ligation can include a “gap-filling” step that involves incorporation of one or more nucleic acids by a polymerase, based on the nucleic acid sequence of a template nucleic acid molecule, spanning a distance between the two nucleic acid molecules of interest (see, e.g., U.S. Pat. No. 7,264,929, the entire contents of which are incorporated herein by reference).

A wide variety of different methods can be used for proximity ligating nucleic acid molecules, including (but not limited to) “sticky-end” and “blunt-end” ligations. Additionally, single-stranded ligation can be used to perform proximity ligation on a single-stranded nucleic acid molecule. Sticky-end proximity ligations involve the hybridization of complementary single-stranded sequences between the two nucleic acid molecules to be joined, prior to the ligation event itself. Blunt-end proximity ligations generally do not include hybridization of complementary regions from each nucleic acid molecule because both nucleic acid molecules lack a single-stranded overhang at the site of ligation.

### (x) Nucleic Acid Extension

A “nucleic acid extension” generally involves incorporation of one or more nucleic acids (e.g., A, G, C, T, U, nucleotide analogs, or derivatives thereof) into a molecule (such as, but not limited to, a nucleic acid sequence) in a template-dependent manner, such that consecutive nucleic acids are incorporated by an enzyme (such as a polymerase or reverse transcriptase), thereby generating a newly synthesized nucleic acid molecule. For example, a primer that hybridizes to a complementary nucleic acid sequence can be used to synthesize a new nucleic acid molecule by using the complementary nucleic acid sequence as a template for nucleic acid synthesis. Similarly, a 3′ polyadenylated tail of an mRNA transcript that hybridizes to a poly (dT) sequence (e.g., capture domain) can be used as a template for single-strand synthesis of a corresponding cDNA molecule.

### (xi) PCR Amplification

A “PCR amplification” refers to the use of a polymerase chain reaction (PCR) to generate copies of genetic material, including DNA and RNA sequences. Suitable reagents and conditions for implementing PCR are described, for example, in U.S. Pat. Nos. 4,683,202, 4,683,195, 4,800,159, 4,965,188, and 5,512,462, the entire contents of each of which are incorporated herein by reference. In a typical PCR amplification, the reaction mixture includes the genetic material to be amplified, an enzyme, one or more primers that are employed in a primer extension reaction, and reagents for the reaction. The oligonucleotide primers are of sufficient length to provide for hybridization to complementary genetic material under annealing conditions. The length of the primers generally depends on the length of the amplification domains, but will typically be at least 4 bases, at least 5 bases, at least 6 bases, at least 8 bases, at least 9 bases, at least 10 base pairs (bp), at least 11 bp, at least 12 bp, at least 13 bp, at least 14 bp, at least 15 bp, at least 16 bp, at least 17 bp, at least 18 bp, at least 19 bp, at least 20 bp, at least 25 bp, at least 30 bp, at least 35 bp, and can be as long as 40 bp or longer, where the length of the primers will generally range from 18 to 50 bp. The genetic material can be contacted with a single primer or a set of two primers (forward and reverse primers), depending upon whether primer extension, linear or exponential amplification of the genetic material is desired.

In some embodiments, the PCR amplification process uses a DNA polymerase enzyme. The DNA polymerase activity can be provided by one or more distinct DNA polymerase enzymes. In certain embodiments, the DNA polymerase enzyme is from a bacterium, e.g., the DNA polymerase enzyme is a bacterial DNA polymerase enzyme. For instance, the DNA polymerase can be from a bacterium of the genus Escherichia, Bacillus, Thermophilus, or Pyrococcus.

Suitable examples of DNA polymerases that can be used include, but are not limited to: E. coli DNA polymerase I, Bsu DNA polymerase, Bst DNA polymerase, Taq DNA polymerase, VENT™ DNA polymerase, DEEPVENT™ DNA polymerase, LongAmp® Taq DNA polymerase, LongAmp® Hot Start Taq DNA polymerase, Crimson LongAmp® Taq DNA polymerase, Crimson Taq DNA polymerase, OneTaq® DNA polymerase, OneTaq® Quick-Load® DNA polymerase, Hemo KlenTaq® DNA polymerase, REDTaq® DNA polymerase, Phusion® DNA polymerase, Phusion® High-Fidelity DNA polymerase, Platinum Pfx DNA polymerase, AccuPrime Pfx DNA polymerase, Phi29 DNA polymerase, Klenow fragment, Pwo DNA polymerase, Pfu DNA polymerase, T4 DNA polymerase and T7 DNA polymerase enzymes.

The term “DNA polymerase” includes not only naturally-occurring enzymes but also all modified derivatives thereof, including also derivatives of naturally-occurring DNA polymerase enzymes. For instance, in some embodiments, the DNA polymerase can have been modified to remove 5′-3′ exonuclease activity. Sequence-modified derivatives or mutants of DNA polymerase enzymes that can be used include, but are not limited to, mutants that retain at least some of the functional, e.g. DNA polymerase activity of the wild-type sequence. Mutations can affect the activity profile of the enzymes, e.g. enhance or reduce the rate of polymerization, under different reaction conditions, e.g. temperature, template concentration, primer concentration, etc. Mutations or sequence-modifications can also affect the exonuclease activity and/or thermostability of the enzyme.

In some embodiments, PCR amplification can include reactions such as, but not limited to, a strand-displacement amplification reaction, a rolling circle amplification reaction, a ligase chain reaction, a transcription-mediated amplification reaction, an isothermal amplification reaction, and/or a loop-mediated amplification reaction.

In some embodiments, PCR amplification uses a single primer that is complementary to the 3′ tag of target DNA fragments. In some embodiments, PCR amplification uses a first and a second primer, where at least a 3′ end portion of the first primer is complementary to at least a portion of the 3′ tag of the target nucleic acid fragments, and where at least a 3′ end portion of the second primer exhibits the sequence of at least a portion of the 5′ tag of the target nucleic acid fragments. In some embodiments, a 5′ end portion of the first primer is non-complementary to the 3′ tag of the target nucleic acid fragments, and a 5′ end portion of the second primer does not exhibit the sequence of at least a portion of the 5′ tag of the target nucleic acid fragments. In some embodiments, the first primer includes a first universal sequence and/or the second primer includes a second universal sequence.

In some embodiments (e.g., when the PCR amplification amplifies captured DNA), the PCR amplification products can be ligated to additional sequences using a DNA ligase enzyme. The DNA ligase activity can be provided by one or more distinct DNA ligase enzymes. In some embodiments, the DNA ligase enzyme is from a bacterium, e.g., the DNA ligase enzyme is a bacterial DNA ligase enzyme. In some embodiments, the DNA ligase enzyme is from a virus (e.g., a bacteriophage). For instance, the DNA ligase can be T4 DNA ligase. Other enzymes appropriate for the ligation step include, but are not limited to, Tth DNA ligase, Taq DNA ligase, Thermococcus sp. (strain 9oN) DNA ligase (9oN™ DNA ligase, available from New England Biolabs, Ipswich, Mass.), and Ampligase™ (available from Epicentre Biotechnologies, Madison, Wis.). Derivatives, e.g. sequence-modified derivatives, and/or mutants thereof, can also be used.

In some embodiments, genetic material is amplified by reverse transcription polymerase chain reaction (RT-PCR). The desired reverse transcriptase activity can be provided by one or more distinct reverse transcriptase enzymes, suitable examples of which include, but are not limited to: M-MLV, MuLV, AMV, HIV, ArrayScript™, MultiScribe™, ThermoScript™, and SuperScript® I, II, III, and IV enzymes. “Reverse transcriptase” includes not only naturally occurring enzymes, but all such modified derivatives thereof, including also derivatives of naturally-occurring reverse transcriptase enzymes.

In addition, reverse transcription can be performed using sequence-modified derivatives or mutants of M-MLV, MuLV, AMV, and HIV reverse transcriptase enzymes, including mutants that retain at least some of the functional, e.g. reverse transcriptase, activity of the wild-type sequence. The reverse transcriptase enzyme can be provided as part of a composition that includes other components, e.g. stabilizing components that enhance or improve the activity of the reverse transcriptase enzyme, such as RNase inhibitor(s), inhibitors of DNA-dependent DNA synthesis, e.g. actinomycin D. Many sequence-modified derivative or mutants of reverse transcriptase enzymes, e.g. M-MLV, and compositions including unmodified and modified enzymes are commercially available, e.g. ArrayScript™, MultiScribe™, ThermoScript™, and SuperScript® I, II, III, and IV enzymes.

Certain reverse transcriptase enzymes (e.g. Avian Myeloblastosis Virus (AMV) Reverse Transcriptase and Moloney Murine Leukemia Virus (M-MuLV, MMLV) Reverse Transcriptase) can synthesize a complementary DNA strand using both RNA (cDNA synthesis) and single-stranded DNA (ssDNA) as a template. Thus, in some embodiments, the reverse transcription reaction can use an enzyme (reverse transcriptase) that is capable of using both RNA and ssDNA as the template for an extension reaction, e.g. an AMV or MMLV reverse transcriptase.

In some embodiments, the quantification of RNA and/or DNA is carried out by real-time PCR (also known as quantitative PCR or qPCR), using techniques such as but not limited to “TAQMAN™” or “SYBR®”, or on capillaries (“LightCycler® Capillaries”). In some embodiments, the quantification of genetic material is determined by optical absorbance and with real-time PCR. In some embodiments, the quantification of genetic material is determined by digital PCR. In some embodiments, the genes analyzed can be compared to a reference nucleic acid extract (DNA and RNA) corresponding to the expression (mRNA) and quantity (DNA) in order to compare expression levels of the target nucleic acids.

(xii) Label, Detectable Label, and Optical Label

The terms “detectable label” and “label” are used interchangeably herein to refer to a directly or indirectly detectable moiety that is associated with (e.g., conjugated to) a molecule to be detected, e.g., a probe for in situ assay, or analyte. The detectable label can be directly detectable by itself (e.g., radioisotope labels or optical labels such as fluorescent labels) or, in the case of an enzymatic label, can be indirectly detectable, e.g., by catalyzing chemical alterations of a substrate compound or composition, which substrate compound or composition is directly detectable. Detectable labels can be suitable for small scale detection and/or suitable for high-throughput screening. As such, suitable detectable labels include, but are not limited to, radioisotopes, fluorophores, chemiluminescent compounds, bioluminescent compounds, and dyes.

The detectable label can be qualitatively detected (e.g., optically or spectrally), or it can be quantified. Qualitative detection generally includes a detection method in which the existence or presence of the detectable label is confirmed, whereas quantifiable detection generally includes a detection method having a quantifiable (e.g., numerically reportable) value such as an intensity, duration, polarization, and/or other properties. In some embodiments, the detectable label is bound to a feature. For example, detectably labeled features can include a fluorescent, a colorimetric, or a chemiluminescent label attached to an analyte, probe, or bead (see, for example, Rajeswari et al., J. Microbiol Methods 139:22-28, 2017, and Forcucci et al., J. Biomed Opt. 10:105010, 2015, the entire contents of each of which are incorporated herein by reference).

In some embodiments, a plurality of detectable labels can be attached to a feature, probe, or composition to be detected. For example, detectable labels can be incorporated during nucleic acid polymerization or amplification (e.g., Cy5®-labelled nucleotides, such as Cy5®-dCTP). Any suitable detectable label can be used. In some embodiments, the detectable label is a fluorophore.

As mentioned above, in some embodiments, a detectable label is or includes a luminescent or chemiluminescent moiety. Common luminescent/chemiluminescent moieties include, but are not limited to, peroxidases such as horseradish peroxidase (HRP), soybean peroxidase (SP), alkaline phosphatase, and luciferase. These protein moieties can catalyze chemiluminescent reactions given the appropriate substrates (e.g., an oxidizing reagent plus a chemiluminescent compound. A number of compound families can provide chemiluminescence under a variety of conditions. Non-limiting examples of chemiluminescent compound families include 2,3-dihydro-1,4-phthalazinedione luminol, 5-amino-6,7,8-trimethoxy- and the dimethylamino[ca]benz analog. These compounds can luminesce in the presence of alkaline hydrogen peroxide or calcium hypochlorite and base. Other examples of chemiluminescent compound families include, e.g., 2,4,5-triphenylimidazoles, para-dimethylamino and -methoxy substituents, oxalates such as oxalyl active esters, p-nitrophenyl, N-alkyl acridinum esters, luciferins, lucigenins, or acridinium esters. In some embodiments, a detectable label is or includes a metal-based or mass-based label. For example, small cluster metal ions, metals, or semiconductors may act as a mass code. In some examples, the metals can be selected from Groups 3-15 of the periodic table, e.g., Y, La, Ag, Au, Pt, Ni, Pd, Rh, Ir, Co, Cu, Bi, or a combination thereof.

### Exemplary Embodiments

1. A method for analyzing a biological sample comprising a plurality of analytes, comprising:

(a) detecting a plurality of optical signals in sequential cycles, wherein at least a subset of the plurality of optical signals detected at a location in the biological sample form an optical signature corresponding to an analyte of the plurality of analytes, the detecting comprising:
i) in a first cycle, detecting a plurality of overlapping optical signals at a location;
ii) in a second cycle, detecting a first non-overlapping optical signal at the location, wherein the first non-overlapping optical signal is associated with a first analyte; and
iii) in a third cycle, detecting a second non-overlapping optical signal at the location, wherein the second non-overlapping optical signal is associated with a second analyte; and

(b) generating a plurality of potential signal sequence chains comprising optical signals from each of the cycles from (a); and

(c) using an identifier for the first analyte and an identifier for the second analyte to associate a first potential signal sequence chain of the plurality of potential signal sequence chains to the first analyte, and associate a second potential signal sequence chain of the plurality of potential signal sequence chains to the second analyte,

wherein the identifier for the first analyte is a first order of signal codes that identifies the first analyte, and the identifier for the second analyte is a second order of signal codes that identifies the second analyte, and

wherein the first order comprises signal codes that match an optical signature comprising a first overlapping optical signal of the plurality of overlapping optical signals and the first non-overlapping optical signal, and the second order comprises signal codes that match an optical signature comprising a second overlapping optical signal of the plurality of overlapping optical signals and the second non-overlapping optical signal,

thereby identifying the first and second analytes at the location.

2. The method of embodiment 1, wherein a probability of matching the identifier for the first or second analyte is assigned to each potential signal sequence chain of the plurality of potential signal sequence chains.

3. The method of embodiment 2, wherein step (c) comprises comparing the plurality of potential signal sequence chains to the identifier for the first analyte and the identifier for the second analyte to assign the probability.

4. The method of any of embodiments 1-3, wherein the plurality of potential signal sequence chains generated in step (b) comprise the presence and absence of signals detected from step (a).

5. The method of any of embodiments 1-4, wherein step (b) comprises associating the first non-overlapping optical signal at the location with the first overlapping optical signal of the plurality of overlapping optical signals at the location to generate the first potential signal sequence chain.

6. The method of any of embodiments 1-5, wherein step (b) comprises associating the second non-overlapping optical signal at the location with the second overlapping optical signal of the plurality of overlapping optical signals at the location to generate the second potential signal sequence chain.

7. The method of any of embodiments 1-6, wherein the optical signals are detected by detecting detectable probes targeting the plurality of analytes.

8. The method of any of embodiments 1-7, wherein the optical signals are detected by imaging the biological sample using fluorescent microscopy.

9. The method of any of embodiments 1-8, wherein the optical signals are detected in situ in the biological sample.

10. The method of any of embodiments 1-9, wherein the optical signals are detected by:

contacting the biological sample with one or more detectably-labeled probes that directly or indirectly hybridize to nucleic acid sequences in or associated with the plurality of analytes, and

dehybridizing the one or more detectably-labeled probes from the nucleic acid sequences,

optionally wherein the contacting and dehybridizing steps are repeated with the one or more detectably-labeled probes and/or one or more other detectably-labeled probes that directly or indirectly hybridize to nucleic acid sequences in or associated with the plurality of analytes.

11. The method of any of embodiments 1-9, wherein the optical signals are detected by:

contacting the biological sample with one or more intermediate probes that directly or indirectly hybridize to nucleic acid sequences in or associated with the plurality of analytes, wherein the one or more intermediate probes are detectable using one or more detectably-labeled probes, and

dehybridizing the one or more intermediate probes and/or the one or more detectably-labeled probes from the nucleic acid sequences,

optionally wherein the contacting and dehybridizing steps are repeated with the one or more intermediate probes, the one or more detectably-labeled probes, one or more other intermediate probes, and/or one or more other detectably-labeled probes.

12. The method of embodiment 11, wherein the one or more intermediate probes each comprises a sequence that hybridizes to one of the nucleic acid sequences and one or more overhangs that hybridize to a detectably-labeled probe but not to the nucleic acid sequences.

13. The method of any of embodiments 1-12, wherein in the second cycle, detecting the first non-overlapping optical signal associated with the first analyte comprises omitting a selected detectable probe targeting the second analyte and/or blocking the selected detectable probe from targeting the second analyte.

14. The method of any of embodiments 1-13, wherein in the third cycle, detecting the second non-overlapping optical signal associated with the second analyte comprises omitting a selected detectable probe targeting the first analyte and/or blocking the selected detectable probe from targeting the first analyte.

15. The method of embodiment 13 or 14, wherein blocking the selected detectable probe from targeting the first or second analyte comprises contacting the sample with an interfering agent, wherein the interfering agent interferes with hybridization of the selected detectable probe to its corresponding nucleic acid sequence in or associated with the corresponding analyte.

16. The method of embodiment 15, wherein the selected detectable probe is contacted with the interfering agent to form a detectable probe/interfering agent hybridization complex, before the sample is contacted with the selected detectable probe.

17. The method of embodiment 15, wherein the sample is contacted with the interfering agent to form a hybridization complex between the interfering agent and the nucleic acid sequence in or associated with the corresponding analyte.

18. The method of any of embodiments 15-17, wherein the method comprises contacting the sample with a plurality of interfering agents.

19. The method of embodiment 15, wherein the interfering agent displaces the selected detectable probe which is hybridized to the nucleic acid sequence in or associated with the corresponding analyte.

20. The method of any of embodiments 1-19, wherein the second cycle comprises contacting the sample with an interfering agent that blocks hybridization and/or detection of the second analyte, and/or the third cycle comprises contacting the sample with an interfering agent that blocks hybridization and/or detection of the first analyte.

21. The method of any of embodiments 15-20, wherein the selected detectable probe comprises one or more overhangs that do not hybridize the nucleic acid sequence in or associated with the corresponding analyte.

22. The method of embodiment 21, wherein at least one of the one or more overhangs is capable of hybridizing to a detectably labelled detection probe.

23. The method of any one of embodiments 15-22, wherein the selected detectable probe comprises an overhang that is capable of hybridizing to a sequence of the interfering agent.

24. The method of embodiment 23, wherein hybridization of the interfering agent to the overhang of the selected detectable probe initiates a strand displacement reaction whereby the interfering agent hybridizes to the selected detectable probe and displaces it from the corresponding nucleic acid sequence.

25. The method of embodiment 15, wherein the interfering agent hybridizes to the nucleic acid sequence corresponding to the selected detectable probe and prevents, competes with, and/or displaces the selected detectable probe(s) from hybridizing to the nucleic acid sequence.

26. The method of any of embodiments 15-25, wherein the interfering agent is provided at a higher concentration than probe(s) for the target nucleic acid sequence.

27. The method of any of embodiments 15-26, wherein the interfering agent comprises a quencher moiety.

28. The method of embodiment 13 or 14, wherein the blocking the detectable probe from targeting the second or first analyte comprises (i) directly or indirectly binding a probe to the detectable probe, thereby blocking the detectable probe from directly or indirectly binding to the second or first analyte, or (ii) directly or indirectly binding a probe to the second or first analyte, thereby blocking the second or first analyte from directly or indirectly binding to the detectable probe.

29. The method of any of embodiments 1-28, wherein the first cycle is before or after the second cycle, optionally wherein the first and second cycles are consecutive or separated by one or more other cycles.

30. The method of any of embodiments 1-29, wherein the first cycle is before or after the third cycle, optionally wherein the first and third cycles are consecutive or separated by one or more other cycles.

31. The method of any of embodiments 1-30, wherein the second cycle is before or after the third cycle, optionally wherein the second and third cycles are consecutive or separated by one or more other cycles.

32. The method of any of embodiments 1-31, wherein in the second cycle, an absence of optical signal associated with the second analyte is detected at the location.

33. The method of embodiment 32, wherein step (b) comprises associating the absence of optical signal at the location with the second overlapping optical signal of the plurality of overlapping optical signals at the location to generate the second potential signal sequence chain.

34. The method of embodiment 33, wherein the second potential signal sequence chain comprises the second overlapping optical signal, the absence of optical signal in the second cycle, and the second non-overlapping optical signal.

35. The method of any of embodiments 1-34, wherein in the third cycle, an absence of optical signal associated with the first analyte is detected at the location.

36. The method of embodiment 35, wherein step (b) comprises associating the absence of optical signal at the location with the first overlapping optical signal of the plurality of overlapping optical signals at the location to generate the first potential signal sequence chain.

37. The method of embodiment 36, wherein the first potential signal sequence chain comprises the first overlapping optical signal, the first non-overlapping optical signal, and the absence of optical signal in the third cycle.

38. The method of any of embodiments 1-37, wherein the plurality of overlapping optical signals detected in the first cycle is a first plurality of overlapping optical signals, and the detecting in step a) further comprises:

iv) in a fourth cycle, detecting a second plurality of overlapping optical signals at the location.

39. The method of any of embodiments 1-38, wherein the sequential cycles comprise a fifth cycle in which no optical signal associated with the first analyte and no optical signal associated with the second analyte is detected at the location.

40. The method of embodiment 39, wherein in the fifth cycle, an optical signal associated with a third analyte is detected at the location.

41. The method of any of embodiments 1-40, wherein the plurality of optical signals are associated with detectable probes targeting at least a subset of the plurality of analytes in each cycle.

42. The method of embodiment 41, wherein the detectable probes each comprises a fluorophore.

43. The method of embodiment 41 or 42, wherein the detectable probes each comprises an analyte targeting region.

44. The method of embodiment 43, wherein two or more detectable probes targeting the same analyte comprise the same analyte targeting region or different analyte targeting regions.

45. The method of any of embodiments 41-44, wherein the detectable probes each comprises a probe binding region that binds to a fluorescently labelled probe.

46. The method of embodiment 45, wherein two or more detectable probes targeting the same analyte comprise the same probe binding region or different probe binding regions.

47. The method of embodiment 45 or 46, wherein two or more detectable probes targeting the same analyte bind to the same fluorescently labelled probe or different fluorescently labelled probes.

48. A method for analyzing a biological sample comprising a plurality of analytes, comprising:

(a) in sequential cycles, contacting the biological sample with a plurality of detectable probes each comprising an analyte targeting region,

wherein in the sequential cycles, detectable probes targeting a particular analyte are contacted with the biological sample according to an order of signal codes in an identifier that identifies that particular analyte among the plurality of analytes, the signal codes corresponding to optical signals associated with the detectable probes, and

wherein the plurality of detectable probes comprise a first probe set targeting a first analyte and a second probe set targeting a second analyte, and the sequential cycles comprise:
(i) one or more non-sparse cycles in which the biological sample is contacted with a detectable probe of the first probe set and a detectable probe of the second detectable probe set; and
(ii) one or more sparse cycles in which the biological sample is contacted with a detectable probe of the first probe set and no detectable probe of the second probe set, or vice versa;

(b) in the sequential cycles, detecting optical signals associated with the detectable probes at a location in the biological sample to provide a plurality of potential signal sequence chains comprising the detected optical signals for analytes at the location,

wherein in a non-sparse cycle, optical signals associated with the first and second analytes are overlapping at the location, resulting in an ambiguity in analyte identity at the location, and

wherein in a sparse cycle, the optical signal associated with the first analyte at the location does not overlap with an optical signal associated with the second analyte, or vice versa; and

(c) comparing the plurality of potential signal sequence chains for analytes at the location to the identifiers for the plurality of analytes to identify a match, thereby associating the overlapping optical signals at the location to the first analyte and the second analyte, respectively, thereby resolving the ambiguity in the non-sparse cycle,

thereby identifying the first and second analytes at the location.

49. A method for analyzing a biological sample comprising a plurality of analytes, comprising:

(a) in sequential cycles, contacting the biological sample with a plurality of detectable probes each comprising an analyte targeting region and a fluorescently labelled probe binding region,

wherein in the sequential cycles, detectable probes targeting a particular analyte are contacted with the biological sample according to an order of signal codes in an identifier that identifies that particular analyte among the plurality of analytes, the signal codes corresponding to optical signals associated with the fluorescently labelled probe binding regions of the detectable probes, and

wherein the plurality of detectable probes comprise a first probe set targeting a first analyte and a second probe set targeting a second analyte, and the sequential cycles comprise:
(i) one or more non-sparse cycles in which the biological sample is contacted with a detectable probe of the first probe set and a detectable probe of the second detectable probe set; and
(ii) one or more sparse cycles in which the biological sample is contacted with a detectable probe of the first probe set and no detectable probe of the second probe set, or vice versa;

(b) in the sequential cycles, contacting the biological sample with fluorescently labelled probes each capable of binding to the fluorescently labelled probe binding region of a detectable probe in that particular cycle;

(c) detecting optical signals associated with the fluorescently labelled probes at a location in the biological sample to provide a plurality of potential signal sequence chains comprising the detected optical signals for analytes at the location,

wherein in a non-sparse cycle, optical signals associated with the first and second analytes are overlapping at the location, resulting in an ambiguity in analyte identity at the location, and

wherein in a sparse cycle, the optical signal associated with the first analyte at the location does not overlap with an optical signal associated with the second analyte, or vice versa; and

(c) comparing the plurality of potential signal sequence chains for analytes at the location to the identifiers for the plurality of analytes to identify a match, thereby associating the overlapping optical signals at the location to the first analyte and the second analyte, respectively, thereby resolving the ambiguity in the non-sparse cycle,

thereby identifying the first and second analytes at the location.

50. The method of embodiment 49, wherein an optical signal corresponding to the sparse cycle is removed as a result of the match at the location.

51. The method of embodiment 49 or 50, wherein the location is a first location, and wherein at a second location in the biological sample, optical signals associated with the first and second analytes are non-overlapping in the non-sparse cycle.

52. The method of embodiment 51, wherein optical signals associated with the first and second analytes are non-overlapping at the second location in the sparse cycle.

53. The method of embodiment 51 or 52, wherein the plurality of potential signal sequence chains at the second location comprise optical signals of the non-sparse cycle and the sparse cycle.

54. The method of embodiment 53, wherein at the second location, an optical signal associated with a detectable probe is not detected, and the absence of optical signal is recorded as part of the plurality of potential signal sequence chains.

55. The method of embodiment 54, wherein the plurality of potential signal sequence chains at the second location are compared to the identifiers for the plurality of analytes to identify a match.

56. The method of embodiment 55, wherein the recorded absence of optical signal is removed as the result of the match at the second location.

57. The method of any of embodiments 49-56, wherein the non-sparse cycle is before or after the sparse cycle.

58. The method of any of embodiments 49-57, wherein the non-sparse cycle and the sparse cycle are consecutive or separated by one or more other cycles.

59. The method of any of embodiments 49-58, wherein in the non-sparse cycle, the biological sample is contacted with a detectable probe targeting each of the plurality of analytes.

60. The method of any of embodiments 49-59, wherein in the sparse cycle, the biological sample is contacted with a detectable probe targeting each of the plurality of analytes except the first analyte or the second analyte.

61. The method of any of embodiments 49-60, wherein the sequential cycles comprise two, three, four, five, six, or more non-sparse cycles for the first and second analytes.

62. The method of any of embodiments 49-61, wherein the sequential cycles comprise two, three, four, five, six, or more sparse cycles for the first analyte and/or the second analyte.

63. The method of any of embodiments 49-62, wherein two, three, four, five, six, or more of the detectable probes targeting the particular analyte comprise different fluorescently labelled probe binding regions.

64. The method of embodiment 63, wherein the plurality of detectable probes comprise four different fluorescently labelled probe binding regions each capable of hybridizing to a fluorescently labelled probe comprising a different fluorophore.

65. The method of any of embodiments 49-64, wherein the first probe set and the second probe set comprise detectable probes comprising the same fluorescently labelled probe binding region.

66. The method of any of embodiments 49-65, wherein the first probe set and the second probe set comprise detectable probes comprising different fluorescently labelled probe binding regions.

67. The method of any of embodiments 1-66, comprising three, four, five, six, or more sequential cycles.

68. The method of any of embodiments 1-67, comprising identifying at least 10, at least 25, at least 50, at least 100, at least 250, at least 500, at least 1,000, at least 2,500, or more different analytes.

69. The method of any of embodiments 1-68, wherein the detecting steps are performed in situ in the biological sample.

70. The method of any of embodiments 1-69, wherein the plurality of analytes comprise nucleic acid analytes and/or protein analytes.

71. The method of embodiment 70, wherein the nucleic acid analytes comprise rolling circle amplification (RCA) products.

72. The method of any of embodiments 1-71, wherein analytes at at least 10, at least 50, at least 100, at least 500, at least 1,000, at least 5,000, at least 10,000, or more different locations in the biological sample are analyzed.

73. A method for analyzing a biological sample comprising a plurality of analytes, comprising:

(a) in sequential cycles, contacting the biological sample with a plurality of detectable probes, wherein the biological sample comprises multiple rolling circle amplification (RCA) products each comprising a target nucleic acid sequence corresponding to an analyte,

wherein each detectable probe comprises (i) a target hybridizing region that hybridizes to the target nucleic acid sequence and (ii) a fluorescently labelled probe hybridizing region,

wherein in the sequential cycles, detectable probes for a particular analyte are contacted with the biological sample according to an identifier comprising an order of signal codes that identifies that particular analyte among the plurality of analytes, the signal codes corresponding to optical signals from the fluorescently labelled probes that hybridize to the detectable probes, and

wherein the plurality of detectable probes comprise a first probe set for a first analyte and a second probe set for a second analyte, and the sequential cycles comprise:
(i) one or more non-sparse cycles in which the biological sample is contacted with a detectable probe of the first probe set and a detectable probe of the second detectable probe set; and
(ii) one or more sparse cycles in which the biological sample is contacted with a detectable probe of the first probe set and no detectable probe of the second probe set, or vice versa;

(b) in the sequential cycles, contacting the biological sample with fluorescently labelled probes each capable of hybridizing a detectable probe in that particular cycle;

(c) detecting optical signals associated with the fluorescently labelled probes at a location in the biological sample to provide a plurality of potential signal sequence chains comprising the detected optical signals for analytes at the location,

wherein in a non-sparse cycle, optical signals associated with the first and second analytes are overlapping at the location, resulting in an ambiguity in analyte identity at the location, and

wherein in a sparse cycle, the optical signal associated with the first analyte at the location does not overlap with an optical signal associated with the second analyte, or vice versa; and

(d) comparing the plurality of potential signal sequence chains for analytes at the location to the identifiers for the plurality of analytes to identify a match, thereby associating the overlapping optical signals at the location to the first analyte and the second analyte, respectively, thereby resolving the ambiguity in the non-sparse cycle,

thereby identifying the first and second analytes at the location.

74. The method of embodiment 73, wherein the target nucleic acid sequence is a barcode sequence or complement thereof.

75. The method of embodiment 74, wherein each RCA product comprise multiple copies of the barcode sequence or complement thereof.

76. The method of any of embodiments 73-75, wherein at least three, four, five, six, or more of the plurality of detectable probes comprise the same target hybridizing region but different fluorescently labelled probe hybridizing regions.

77. The method of any of embodiments 73-76, wherein at least three, four, five, six, or more of the plurality of detectable probes comprise different target hybridizing regions but the same fluorescently labelled probe hybridizing region.

78. The method of any of embodiments 73-77, wherein the multiple RCA products are generated and detected in situ.

79. The method of any of embodiments 73-78, wherein the multiple RCA products are generated using a circular or circularizable probe or probe set that hybridizes to:

(i) a nucleic acid analyte in the biological sample;

(ii) a product of a nucleic acid analyte in the biological sample;

(iii) a reporter oligonucleotide of a labelling agent that directly or indirectly binds to a nucleic acid analyte or a non-nucleic acid analyte in the biological sample; or

(iv) a product of a reporter oligonucleotide of a labelling agent that directly or indirectly binds to a nucleic acid analyte or a non-nucleic acid analyte in the biological sample.

80. The method of embodiment 79, wherein the nucleic acid analyte is an mRNA and/or the non-nucleic acid analyte is a protein.

81. A method for analyzing a biological sample comprising a plurality of analytes, comprising:

in sequential cycles, contacting the biological sample with a plurality of detectable probes each comprising an analyte targeting region,

wherein in the sequential cycles, detectable probes targeting a particular analyte are contacted with the biological sample according to an order of signal codes in an identifier that identifies that particular analyte among the plurality of analytes, the signal codes corresponding to signals associated with the detectable probes, and

wherein the plurality of detectable probes comprise a first probe set targeting a first analyte and a second probe set targeting a second analyte, and the sequential cycles comprise one or more sparse cycles in which the biological sample is contacted with:

(i) a detectable probe of the first probe set, a detectable probe of the second probe set, and an interfering agent that blocks hybridization and/or detection of the detectable probe of the second probe set, wherein a signal associated with a detectable probe of the first probe set is detected and a signal associated with a detectable probe of the second probe set is not detected, or

(ii) a detectable probe of the first probe set, a detectable probe of the second probe set, and an interfering agent that blocks hybridization and/or detection of the detectable probe of the first probe set, wherein a signal associated with a detectable probe of the second probe set is detected and a signal associated with a detectable probe of the first probe set is not detected;

thereby determining a sequential sequence of signal codes that identify the first analyte and the second analyte.

82. The method of embodiment 81, wherein the sequential cycles comprise one or more non-sparse cycles in which the biological sample is contacted with a detectable probe of the first probe set and a detectable probe of the second detectable probe set in the absence of an interfering agent; and

wherein in a non-sparse cycle, optical signals associated with the first and second analytes are overlapping at the location, resulting in an ambiguity in analyte identity at the location, and

wherein in a sparse cycle, the optical signal associated with the first analyte at the location does not overlap with an optical signal associated with the second analyte, or vice versa; and

(d) comparing the plurality of potential signal sequence chains for analytes at the location to the identifiers for the plurality of analytes to identify a match, thereby associating the overlapping optical signals at the location to the first analyte and the second analyte, respectively, thereby resolving the ambiguity in the non-sparse cycle,

thereby identifying the first and second analytes at the location.

83. The method of any of embodiments 1-82, wherein the biological sample is a processed or cleared tissue sample.

84. The method of any of embodiments 1-83, wherein the biological sample is a tissue slice between about 1μm and about 50 μm in thickness, optionally wherein the tissue slice is between about 5 μm and about 35 μm in thickness.

85. The method of any of embodiments 1-84, wherein the biological sample is embedded in a hydrogel.

86. The method of any of embodiments 1-85, wherein the biological sample is not embedded in a hydrogel.

87. The method of any of embodiments 1-86, wherein the biological sample is cross-linked.

### EXAMPLE

The following example is included for illustrative purposes only and is not intended to limit the scope of the present disclosure.

### Example 1

### Sparse Labelling Read-Out of a 10-Plex In Situ Sequencing (ISS) Reaction

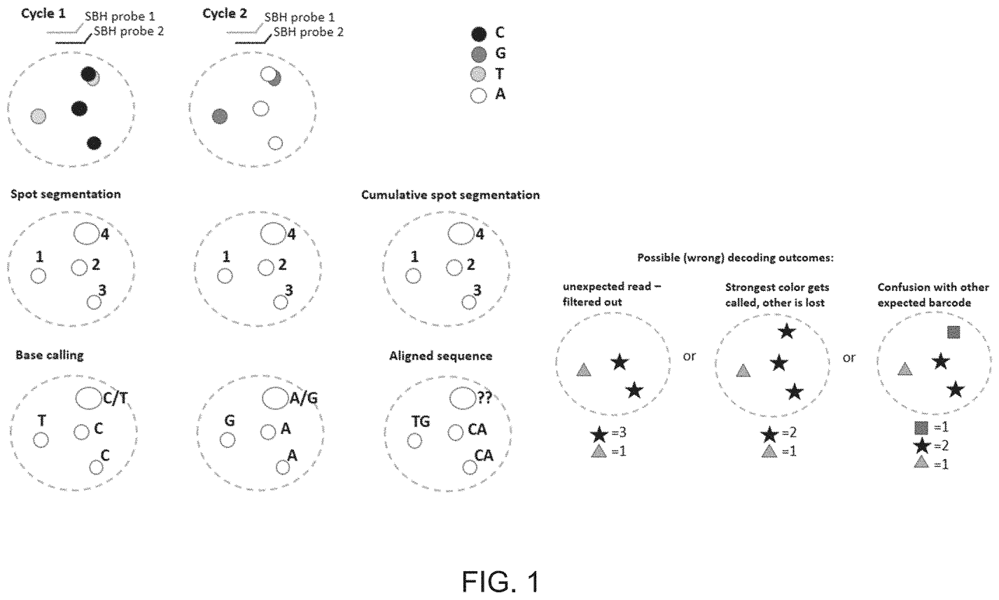

This example illustrates an exemplary workflow applying sparse labelling during detection of a plurality of analytes. While analyzing a tissue section with a 10-plex padlock probe panel targeting 10 medium-high expressed genes using sequencing-by-hybridization, it was observed where signals corresponding to some of the highly expressed genes overlapped/co-occurred. In some cells in the sample, multiple signals were being generated and it was difficult to optically resolve individual RCA products. This lack of resolution inhibited the decoding cycles of the sequencing-by-hybridization (SBH) read out scheme (FIG. 1). FIG. 1 shows a schematic illustrating typical problems which occur with molecular and optical crowding when using a reference combinatorial read out scheme that utilizes the full set of hybridization probes for all analytes to be detected for each cycle. These can result in poor resolution, leading to signal loss and/or barcode misinterpretation.

FIG. 2 shows a schematic illustrating the detection of the same overlapping signals using a sparse labelling approach, which leads to higher resolution, increased dynamic range and increased accuracy of sequencing reads. The signal code sequences for each target are made up of three signal codes but cycles 2 and 3 are dark cycles as they omit hybridization probes (e.g., SBH probes 2 and 1 respectively), and thus 4 cycles are required in total. The dark cycles cause the signals from the omitted probes to ‘drop out’ of the detection scheme, and thus make the other signals more visible. In the decoding step, the dark cycles are deduced and the signal code sequences (without the dark cycles included) are matched to the most likely barcode to identify the detected gene at each position. Compared to the workflow shown in FIG. 1, the hybridization probes which were used for the SBH reaction were unchanged, but they were applied in a new scheme, such that the optical signals which were generated for each gene were interspersed with dark cycles in which certain RCA products were not detected. This scheme is illustrated in Table 2.

TABLE 2
Sparse labelling decoding scheme
for example of 10-plex assay.

| Codes with | | | Reduced to | |
|---|---|---|---|---|
| dark spacers: | | dark spacers | coding signals: | |
| Code | gene | in cycles | Code | gene |
| GXGTXC | Calb2 | 2 & 5 | GGTC | calb2 |
| GCXTGX | Chodl | 3 & 6 | GCTG | chodl |
| TCGXTX | Cort | 4 & 6 | TCGT | cort |
| XTTCAX | NeuroD6 | 1 & 6 | TTCA | neurod6 |
| XGTXAA | PCP4 | 1 & 4 | GTAA | pcp4 |
| XAXTGA | Penk | 1 & 3 | ATGA | penk |
| CXGTXG | Plcxd2 | 2 & 5 | CGTG | plcxd2 |
| GAXGTX | Rorb | 3 & 6 | GAGT | rorb |
| TTCXXT | Rprm | 4 & 5 | TTCT | rprm |
| AXTCXA | Nov | 2 & 5 | ATCA | nov |

Table 2 shows the design of probe pools for the 6 cycles including 2 dark spacer rounds for each gene for the exemplary experiment. The gene column indicates the target of the hybridization probes. The code column comprises the signal code sequences for each gene, including indications as to the timing of the dark cycles for each gene, in which the respective gene will not generate a detectable signal. “X” denotes the omission of a hybridization probe specific to that gene in the pool. The right side of the table shows the signal code sequence without the dark cycles. It is noted that only the presence of a detected signal participates in the final decoding. The dark cycle indicators do not form part of the signal code sequence and are removed.

### Mouse Tissue Section Preparation

Mouse strain C57BL/6 at 30 days age (P30) was euthanized and the olfactory bulb was dissected via cryosectioning. Cryosectioning was performed on ThermoFisher cryostat, at 10 μm thickness. Sections were then adhered onto ThermoFisher Superfrost glass slides and stored at −70 C until processing.

### Chimeric Padlock Ligation and RCA In Situ

### Fixation and Permeabilization

The tissue slides were removed from −70° C. storage and allowed to thaw for 5 min at room temperature (RT). Fixation was then performed by incubating the slides in 3.7% paraformaldehyde (PFA) in 1× DEPC-PBS at RT. The slides were then washed in 1× DEPC-PBS to ensure that the PFA was completely removed before the permeabilization step. The tissue sections were then permeabilized and subsequently quickly washed twice in 1× DEPC-PBS. Following this, the slides were then dehydrated with an ethanol series before the slides were air-dried. A Secure Seal Chamber (Grace Bio Labs) was applied to each section and the sections were rehydrated with 1× DEPC-PBS-T before continuing with the reverse transcription step.

### Probe Hybridization, Ligation and Rolling Circle Amplification

To prepare for probe hybridization, a wash buffer was added to the chamber and a reaction mixture was prepared by adding a chimeric padlock probe pool together in a hybridization buffer including SSC and formamide. The wash buffer was then removed from the chamber and the prepared reaction mixture was added to the secure seal chamber. The slide was placed in an RNAse free humidity chamber and allowed to incubated.

The reaction mixture containing probes was removed from the chamber and the section was washed and incubated with wash buffer several times. After the last wash in the chamber, a ligation mixture containing T4 RNA ligase buffer, RNAse inhibitor and T4 RNA ligase was added to the chamber. The slide was placed in an RNAse free humidity chamber and allowed to incubate with the ligation mixture. The sample was washed twice in PBS-T and a RCA reaction mixture (containing phi29 reaction buffer, dNTPs, phi29 polymerase) was added and incubated for rolling circle amplification (RCA).

### In Situ Sequencing of RCPs in Tissue Sections Using the Sparse Labeling Approach

### Probe Pool Design

For reference, a sequencing reaction without the presence of dark cycles uses 4 decoding cycles to sequence and therefore identify 10 genes. For the sparse labelling set-up, 6 sequencing cycles were performed, as set out in Table 2 above.

### Probe Stripping

The tissue section was dehydrated with an ethanol series in 70% and 100% ethanol for 2 min respectively before the slides were air-dried. This step allowed the mounting media to be removed. The section was briefly rehydrated a wash buffer. 100% formamide was then added to the section and washed twice. After this wash step, the section was dehydrated with an ethanol series in 70% and 100% ethanol before the slides were air-dried. The samples were then ready to be contacted with the hybridization probes.

### Hybridization and Detection Probe Hybridization

The sections were rehydrated with 1× PBS and the hybridization probe mixtures with probes (e.g., L-shaped probes) each with a region for hybridizing to the target sequence of the corresponding RCA product and an overhang for binding detection probes (e.g., fluorescently labelled detection oligos) in a hybridization buffer with SSC and formamide. The sections were incubated with the hybridization probe mixture and then washed twice with PBS.

After the hybridization, a mixture of detection probes was added in basic hybridization buffer and allowed to hybridize. The sections were then washed twice with basic washing buffer before dehydrating the sections with an ethanol series in 70% and 100% ethanol for 2 min respectively before the slides were air-dried. 10 μl SlowFade Gold antifade reagent (Invitrogen) was then added to each section and covered with a coverslip. The slide was subjected to microscope imaging. After the imaging step, the sections were subjected to a probe stripping step to remove the probes and the next hybridization cycle was performed.

### Decoding of Images

RCA products were identified from individual channels in different cycles. For each RCP, its nearest neighbors within 1 μm distance across all images were identified, only the closest neighbor from each image cycle was kept. A plurality of potential signal sequence chains comprising optical signals from each of the cycles were generated based on the imaging data. Optical signals in the sequential cycles from the individual channels were used to construct a “chained” event for each object which includes clearly detected signals and uncertain (?) signals. For example, a given RCP may have the chained sequence: cycle1:channel4 -cycle2:channel1-c3:?-cycle4:channel2-cycle5:?-cycle6:channel3). During the “chaining” process, all sequence chain possibilities were kept. An automated algorithm was then used to compare all of the possible sequence chains with the existing barcode sequences (e.g., identifiers). The algorithm deduces the dark cycles from the sequence chain and determines the highest likely match to provide a final barcode sequence that matches the signal code sequence with a given % of probability. It was therefore possible to disentangle ambiguous chains that were the result of 2 optically overlapping objects with different barcode sequence, as these could be distinguished as two separate barcodes.

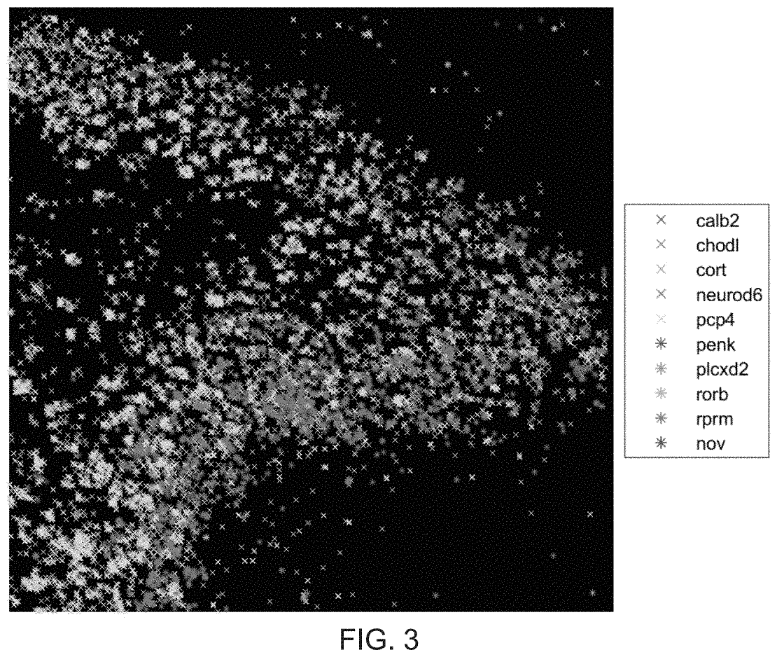

The sparse labelling decoding scheme resulted in a “detection drop-out” of RCA products in the designated dark cycles, which confirmed the specificity of the approach. In those dark cycles, in which highly expressed genes “fall-out” of the decoding scheme and the RCA products are not detected, it was possible to resolve and help quantify the other genes that co-occur in the same region, leading to an increased resolution of the decoding scheme compared with a decoding scheme in which all barcode sequences are detected in every round (FIG. 3). FIG. 3 shows a plot of the results of a sparse labelling detection method on a tissue section of a mouse brain sample subjected to a 10-plex in situ sequencing reaction targeting low and high expressed genes that overlap in expression area. The results shown are the final gene mapping results after successful dark cycle deduction and matching of barcode sequences to expected barcode sequences. These results matched well with gene expression patterns recorded by standard combinatorial labelling methods or methods using individual detection rounds. This approach made it possible to optically resolve a high multiplexed in situ sequencing reaction with high expressed genes that would otherwise optically crowd the sample and hinder the combinatorial decoding scheme.

In this example, the scheme was demonstrated on a system involving the detection of 10 different genes using 6 decoding cycles, with 2 dark cycles used for each gene. The benefits of the sparse labelling system will be particularly useful for decoding reactions with 100s to 1000s of different genes of different expression level, as it can provide an increased dynamic range and greater precision. The “sparse labelling” method involves extra decoding cycles due to the insertion of the dark cycles, but the longer experimental time associated with this increased number of cycles leads to an increase in the dynamic range of the method.

### Example 2

### Sparse Labelling Using Interfering Agents

This example describes an exemplary workflow for detection of a plurality of target nucleic acids using interfering agents for sparse labelling.

The method can be performed as described in Example 1 above, wherein instead of omitting probes for a dark cycle, an interfering agent is applied to block hybridization and/or detection of a given probe for the dark cycle. For example, in the design of probe pools for depicted in Table 2, “X” can denote the inclusion of an interfering agent that blocks hybridization and/or detection of the indicated probe. The interfering agent can be any of those described herein. For example, the interfering agent can hybridize to the probe, thus blocking hybridization of the probe to the target nucleic acid. The interfering agent can hybridize to the target nucleic acid, thus blocking hybridization of the probe to the target nucleic acid. In some instances, an interfering agent interferes with detectably labeled probes that bind the hybridization probe. In some examples, hybridization of the interfering agent to the probe or the target nucleic acid can displace the probe from the target nucleic acid, as illustrated schematically in FIGS. 5A-5B. In another example, the interfering agent can comprise a quencher moiety that blocks detection of the probe signal for the given cycle, as illustrated schematically in FIGS. 6A-6B.

Tissue sectioning, probe hybridization, and detection can be performed as described in Example 1.

The present disclosure is not intended to be limited in scope to the particular disclosed embodiments, which are provided, for example, to illustrate various aspects of the disclosure. Various modifications to the compositions and methods described will become apparent from the description and teachings herein. Such variations may be practiced without departing from the true scope and spirit of the disclosure and are intended to fall within the scope of the present disclosure.

1-87. (canceled)

88. A method for analyzing a biological sample comprising a plurality of analytes, comprising:
(a) detecting a plurality of optical signals in sequential cycles, wherein at least a subset of the plurality of optical signals detected at a location in the biological sample form an optical signature corresponding to an analyte of the plurality of analytes, the detecting comprising:
i) in a first cycle, detecting a plurality of overlapping optical signals at a location;
ii) in a second cycle, detecting a first non-overlapping optical signal at the location, wherein the first non-overlapping optical signal is associated with a first analyte; and
iii) in a third cycle, detecting a second non-overlapping optical signal at the location, wherein the second non-overlapping optical signal is associated with a second analyte; and
(b) generating a plurality of potential signal sequence chains comprising optical signals from each of the cycles from (a); and
(c) using an identifier for the first analyte and an identifier for the second analyte to associate a first potential signal sequence chain of the plurality of potential signal sequence chains to the first analyte, and associate a second potential signal sequence chain of the plurality of potential signal sequence chains to the second analyte,
wherein the identifier for the first analyte is a first order of signal codes that identifies the first analyte, and the identifier for the second analyte is a second order of signal codes that identifies the second analyte, and
wherein the first order comprises signal codes that match an optical signature comprising a first overlapping optical signal of the plurality of overlapping optical signals and the first non-overlapping optical signal, and the second order comprises signal codes that match an optical signature comprising a second overlapping optical signal of the plurality of overlapping optical signals and the second non-overlapping optical signal,
thereby identifying the first and second analytes at the location.

89. The method of claim 88, wherein a probability of matching the identifier for the first or second analyte is assigned to each potential signal sequence chain of the plurality of potential signal sequence chains.

90. The method of claim 88, wherein the plurality of potential signal sequence chains generated in step (b) comprise the presence and absence of signals detected from step (a).

91. The method of claim 88, wherein step (b) comprises associating the first non-overlapping optical signal at the location with the first overlapping optical signal of the plurality of overlapping optical signals at the location to generate the first potential signal sequence chain.

92. The method of claim 88, wherein the optical signals are detected by detecting probes targeting the plurality of analytes, wherein the probes comprise detection hybridization regions for binding detectably labeled probes.

93. The method of claim 88, wherein the detectably labeled probes are fluorescently labelled and the optical signals are detected by imaging the biological sample using fluorescent microscopy.

94. The method of claim 88, wherein in the second cycle, detecting the first non-overlapping optical signal associated with the first analyte comprises omitting a selected probe targeting the second analyte or blocking the selected probe from targeting the second analyte.

95. The method of claim 88, wherein in the third cycle, detecting the second non-overlapping optical signal associated with the second analyte comprises omitting a selected probe targeting the first analyte or blocking the selected probe from targeting the first analyte.

96. The method of claim 94, wherein blocking the selected probe from targeting the second analyte comprises contacting the sample with an interfering agent, wherein the interfering agent interferes with binding of the selected probe to its corresponding nucleic acid sequence in or associated with the corresponding analyte.

97. The method of claim 96, wherein the selected probe is contacted with the interfering agent to form a probe/interfering agent hybridization complex, before the sample is contacted with the selected probe.

98. The method of claim 96, wherein the sample is contacted with the interfering agent to form a hybridization complex between the interfering agent and the nucleic acid sequence in or associated with the corresponding analyte.

99. The method of claim 96, wherein the method comprises contacting the sample with a plurality of interfering agents.

100. The method of claim 96, wherein the interfering agent is provided at a higher concentration than probe(s) for the target nucleic acid sequence.

101. The method of claim 88, wherein in the second cycle, an absence of optical signal associated with the second analyte is detected at the location.

102. The method of claim 101, wherein step (b) comprises associating the absence of optical signal at the location with the second overlapping optical signal of the plurality of overlapping optical signals at the location to generate the second potential signal sequence chain.

103. The method of claim 88, comprising identifying at least 50 different analytes.

104. The method of claim 88, wherein the plurality of analytes comprise a plurality of rolling circle amplification (RCA) products.

105. The method of claim 88, wherein the biological sample is a tissue sample.

106. A method for analyzing a biological sample comprising a plurality of analytes, comprising:
in sequential cycles, contacting the biological sample with a plurality of probes each comprising an analyte targeting region,
wherein in the sequential cycles, probes targeting a particular analyte are contacted with the biological sample according to an order of signal codes in an identifier that identifies that particular analyte among the plurality of analytes, the signal codes corresponding to signals associated with the probes, and
wherein the plurality of probes comprise a first probe set targeting a first analyte and a second probe set targeting a second analyte, and the sequential cycles comprise one or more sparse cycles in which the biological sample is contacted with:
(i) a probe of the first probe set, a probe of the second probe set, and an interfering agent that blocks binding and/or detection of the probe of the second probe set, wherein a signal associated with a probe of the first probe set is detected and a signal associated with a probe of the second probe set is not detected, or
(ii) a probe of the first probe set, a probe of the second probe set, and an interfering agent that blocks binding and/or detection of the probe of the first probe set, wherein a signal associated with a probe of the second probe set is detected and a signal associated with a probe of the first probe set is not detected;
thereby determining a sequential sequence of signal codes that identify the first analyte and the second analyte.

107. The method of claim 106, wherein the sequential cycles comprise one or more non-sparse cycles in which the biological sample is contacted with a probe of the first probe set and a probe of the second probe set in the absence of an interfering agent; and
wherein in a non-sparse cycle, optical signals associated with the first and second analytes are overlapping at the location, resulting in an ambiguity in analyte identity at the location, and
wherein in a sparse cycle, the optical signal associated with the first analyte at the location does not overlap with an optical signal associated with the second analyte, or vice versa; and
(d) comparing the plurality of potential signal sequence chains for analytes at the location to the identifiers for the plurality of analytes to identify a match, thereby associating the overlapping optical signals at the location to the first analyte and the second analyte, respectively, thereby resolving the ambiguity in the non-sparse cycle,
thereby identifying the first and second analytes at the location.
